SEQUENCE LISTING (6 sequences)

SEQ ID NO: 1 - DNA, 26 bp, Mus musculus
ctcgaattcgcatcatggccacgtgc

SEQ ID NO: 2 - DNA, 29 bp, Mus musculus
ctatctcgagttaccacacagccccactg

SEQ ID NO: 3 - DNA, 29 bp, Mus musculus
agaacatctgtgggaagactacaccacag

SEQ ID NO: 4 - DNA, 25 bp, Mus musculus
ctgtggtgtagtcttcccacagatg

SEQ ID NO: 5 - DNA, 29 bp, Mus musculus
ctacagcctggagctcctgcacacattac

SEQ ID NO: 6 - DNA, 29 bp, Mus musculus
gtaatgtgtgcaggagctccaggctgtag

(19) United States
(12) Patent Application Publication
(10) Pub. No.: US 2010/0261180 A1
(43) Pub. Date: Oct. 14, 2010

(54) RECOMBINANT PERFORIN, EXPRESSION AND USES THEREOF

(75) Inventors: JOSEPH ALBERT TRAPANI, CAMBERWELL (AU); MARK JOHN SMYTH, VICTORIA (AU)

Correspondence Address: SCHWEGMAN, LUNDBERG & WOESSNER, P.A., P.O. BOX 2938, MINNEAPOLIS, MN, 55402, US

(73) Assignee: PETER MACCALLUM CANCER INSTITUTE, MELBOURNE (AU)

(21) Appl. No.: 12/704,715

(22) Filed: Feb. 12, 2010

Related U.S. Application Data
(63) Continuation of application No. 11/468,432, filed on Aug. 30, 2006, now abandoned.
Application No. 11/468,432 is a continuation of application No. PCT/AU2005/000291, filed on Mar. 1, 2005.

(30) Foreign Application Priority Data
Mar. 1, 2004 (AU) AU2004901114

(51) Int. Cl. C12Q 1/68

(52) U.S. Cl. 435/6

(57) ABSTRACT

The present invention relates to retroviral vectors capable of driving the expression of perforin in a cell and a method of expressing recombinant perforin in a cell. The present invention also relates to recombinant perforin polypeptides and nucleic acid molecules derived therefrom and uses thereof. Also encompassed are screening assays employing such recombinant perforin molecules, compounds identified by the screening assays and uses thereof.

### RELATED APPLICATION

This application is a continuation application of U.S. patent application Ser. No. 11/468,432, filed Aug. 30, 2006, which application is a continuation under 35 U.S.C. 111(a) of International Patent Application No. PCT/AU2005/000291 filed Mar. 1, 2005 and published in English as WO 2005/083098 A1 on Sep. 9, 2005, which claims priority from Australian Patent Application No. 2004901114 filed Mar. 1, 2004, which applications and publication are incorporated herein by reference and made a part hereof.

The present invention relates to retroviral vectors capable of driving the expression of perforin in a cell and a method of expressing recombinant perforin in a cell. The present invention also relates to recombinant perforin polypeptides and nucleic acid molecules derived therefrom and uses thereof. Also encompassed are screening assays employing such recombinant perforin molecules, compounds identified by the screening assays and uses thereof.

### BACKGROUND

Perforin, a membrane-disruptive protein secreted by cells such as cytotoxic T lymphocytes (CTL) and natural killer (NK) cells, is essential for the death of virus-infected or transformed cells targeted for destruction through the granule exocytosis pathway. Numerous studies have shown that perforin-deficient animals and humans are severely immunosuppressed. For example, mice with targeted disruption of both perforin alleles are markedly susceptible to many viruses and other intracellular pathogens, such as Listeria monocytogenes. The rejection of many experimental tumours is also deficient in these animals, and the likelihood of metastatic spread is frequently elevated. Furthermore, greater than 50% of perforin-deficient animals develop spontaneous, highly aggressive B lymphomas with age, indicating a lapse of tumour immune surveillance. The tumours that arise in these animals are easily transplantable into perforin-deficient recipients, but are avidly rejected by syngeneic immunocompetent animals.

In the CTL, perforin is released from the secretory granules with the granzymes, a family of serine proteases that possess pro-apoptotic activity. By contrast with perforin, a considerable functional redundancy exists in the granzymes, despite their quite distinct proteolytic specificities. For instance, mice deficient in both granzymes A and B are abnormally sensitive only to selected viruses such as ectromelia, but are able to reject a range of experimental tumours and the lymphomas that arise spontaneously in perforin-deficient mice. Overall, it can be surmised that perforin is the only granule constituent that is indispensable for all granule-mediated viral and tumor immunity and immune homeostasis.

A syndrome of perforin deficiency has only recently been described in humans, in that about 30% of children presenting with the rare autosomal recessive disorder familial hemophagocytic lymphohistiocytosis (FHL) have been shown to carry mutations in both their perforin alleles. FHL is one subtype of hemophagocytic lymphohistiocytosis (HLH), which also includes various related immune-deficiency disorders occurring sporadically, with no known familial basis. HLH and FHL are generally characterised by a massive and progressive accumulation of activated T lymphocytes and macrophages (histiocytes) in the liver, spleen, lymph nodes and central nervous system, and consequent phagocytosis of erythrocytes and other blood cells.

The cytotoxic cells, particularly the CTL of these children are unable to impart a lethal hit to target cells through the granule pathway. The defective lymphocytes thus fail to clear antigen-presenting cells, resulting in an uncontrolled activation and accumulation of macrophages and an overproduction of inflammatory cytokines, manifested as the clinical syndrome of fever, liver and spleen enlargement and hemophagocytosis in the spleen, liver and bone marrow. Histologically, the CTL and NK cells of these patients generally demonstrate a marked reduction of immunoreactive perforin in their lytic granules, which may reflect either instability of the perforin protein, or increased perforin turnover in response to an immune challenge. Overall, the clinical and pathological findings in HLH or FHL are reminiscent of the increased expansion of virus-specific T cells and antigen-presenting cells, and the inability to down-regulate the immune response seen in perforin-deficient mice infected with a pathogen such as lymphocytic choriomeningitis virus.

Despite its clear importance, the function of perforin remains poorly understood at the molecular and cellular levels. As purified perforin is unable to induce apoptosis, its key role is thought to involve the accurate targeting of the granzymes to the target cell cytosol, where their proteolytic activity induces the cell's apoptotic program. Granzyme B, the most potent pro-apoptotic granzyme, mimics the activity of the caspases by cleaving substrates after specific aspartate residues (Asp-ase activity). Bid, a pro-apoptotic member of the Bcl-2 family, is a particularly important substrate of granzyme B, as truncated Bid can bring about cell death by activating the intrinsic apoptosis pathway, which is centred on mitochondrial disruption. Granzyme A cleaves after basic residues and induces caspase-independent DNA strand nicking, while mouse granzyme C has been shown to disrupt mitochondrial function directly. Purified perforin applied alone at high concentration can also induce target cell lysis, and this form of cell death may also occur under some physiologically relevant conditions.

At the molecular level, very little is known of how perforin achieves its functions. The carboxyl terminus of perforin is predicted to strongly resemble that of the synaptotagmin family of proteins, some of which are involved in vesicular trafficking at neuronal synapses. One elegant study has produced evidence that during its biosynthesis, perforin is cleaved close to its carboxyl terminus by an unknown protease, liberating a short peptide to which is attached a bulky N-linked glycan. This is predicted to permit calcium and lipid binding at the carboxyl terminus, to enable perforin's insertion into the target cell membrane following CTL degranulation. Following a calcium-dependent conformational change, residues 210 to 245 are believed to form an amphipathic helical structure that permits membrane insertion, although the function of another region with resemblance to an epidermal growth factor receptor cysteine-rich domain (residues 375 to 410) is unknown. Synthetic peptides corresponding to the amino terminus have also been shown to possess some intrinsic lytic capacity. However the physiological relevance of this observation is untested.

Thus, given its vital importance in the immune response to viruses and transformed cells, and despite the fact that both murine and human cDNA were independently cloned more than fifteen years ago, perforin's functions remain poorly understood at the molecular and cellular levels. This lack of substantial progress has been mostly attributed to a lack of cell lines capable of synthesising and storing this toxic protein for the purposes of further investigation.

The use of cultured cell lines has greatly assisted investigations into protein functions across a broad range of research disciplines. Perforin's inherent cytotoxicity has created a special need to identify cells equipped with the appropriate self-protective measures to express it without damaging the organelles in which the protein is synthesized, trafficked and later stored. The scarcity of such cell lines has been the major stumbling block for perforin structure-function studies. Numerous attempts, largely unsuccessful have involved using bacterial expression systems to synthesize perforin. Perforin expression in baculovirus-infected insect cells was unreliable due to solubility problems and this methodology has not become broadly used. A mutational analysis of the perforin molecule has therefore never been described.

On examination of the literature, it becomes apparent that few cells have been successfully used in the past for perforin expression. Evidently, CTL and NK cells are the ideal cells capable of perforin synthesis, however few such cell lines exist in culture. Researchers in the field of lymphocyte biology have resorted to using either freshly isolated lymphocytes, cultured lymphocytic tumours or the few cytotoxic lines immortalised by the introduction of oncogenes. In each case, the drawback is the presence of endogenous perforin in these cells which complicates perforin structure/function investigations. It has previously been shown that the expression of human perforin in a mouse CTL cell line, CTLL-R8 interferes with the function of endogenous perforin, which resulted in decreased cytotoxicity of the transfected cell line. Ideally, structure/function studies would require a cell line devoid of perforin expression, but in which perforin (wild type or mutated) might be reintroduced.

The present invention overcomes, or at least alleviates, some of the aforementioned problems of the prior art and in doing so, provides a more efficient and suitable method of recombinantly expressing perforin, or a fragment or variant thereof, in a cell.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

### SUMMARY OF THE INVENTION

A retroviral vector that is capable of driving the expression of a perforin molecule, or a fragment or variant thereof, in a host cell transfected with said vector.

In yet a further aspect of the present invention, there is provided a packaging cell that is capable of producing a retrovirus particle carrying a retroviral vector that is capable of driving the expression of perforin, or a fragment or variant thereof, in a cell.

In a further aspect of the present invention, there is provided a retrovirus particle carrying a retroviral vector that is capable of driving the expression of perforin, or a fragment or variant thereof, in a cell.

In a further aspect of the present invention, there is provided a host cell or cell line transfected with a retroviral vector capable of driving the recombinant expression of perforin, or a fragment or variant thereof, in the cell.

In an aspect of the present invention, there is provided a method of expressing perforin, or a fragment or variant thereof, in a cell, said method comprising transfecting a cell with a retroviral vector capable of driving the recombinant expression of said perforin, or a fragment or variant thereof, in the cell.

In a further aspect, the present invention provides a recombinant perforin molecule, or a fragment or variant thereof, produced by the methods as herein described.

A method of identifying a compound that modulates expression of a perforin molecule, or a fragment or variant thereof, said method comprising the steps of:
providing a cell transfected with a retroviral vector according to the present invention that is capable of driving the expression of perforin, or a fragment or variant thereof in the cell;
exposing the cell to a test compound; and
determining whether the test compound modulates the expression of the perforin molecule, or a fragment or variant thereof, in the cell.

A method of identifying a compound that modulates activity of a perforin molecule, or a fragment or variant thereof, said method comprising the steps of:
providing an isolated perforin molecule, or an isolated fragment or variant thereof, prepared according to a method of the present invention as herein described;
exposing the isolated perforin molecule, or an isolated fragment or variant thereof, to a test compound and a target cell; and
determining whether the test compound modulates the activity of the perforin molecule, or a fragment or variant thereof, upon the target cell.

A method of identifying a compound that modulates activity of a perforin molecule, or a fragment or variant thereof, said method comprising the steps of:
providing a cell which expresses a perforin molecule, or a fragment or variant thereof according to a method of the present invention as herein described;
exposing the cell to a test compound and a target cell; and
determining whether the test compound modulates the activity of the perforin molecule, or a fragment or variant thereof, upon the target cell.

In yet a further aspect of the present invention, there is provided a compound identified by a screening assay as herein described.

In yet another aspect of the present invention, there is provided a pharmaceutical composition comprising a recombinant perforin molecule as herein described, and/or an agonist or antagonist compound identified by the screening assays as herein described, together with a pharmaceutically acceptable carrier, excipient, diluent and/or adjuvant.

In yet a further aspect of the present invention, there is provided a prophylactic or therapeutic method of treating a subject at risk of or susceptible to a disorder or having a disorder associated with undesirable perforin expression and/or activity.

### FIGURES

FIG. 1 illustrates the primary amino acid sequence and cDNA sequence of human perforin, showing putative functional perforin domains as indicated in the colour legend below the sequence. The numerals at right indicate numbering of the nucleotides (small font) and amino acids (large font), starting at the Met1 start codon. Also depicted are some of the perforin gene mutations so far identified in the FHL disorder. Missense mutations are shown in the filled red circles and frameshift or non-sense mutations in empty circles.

FIG. 2 shows a brief outline of method used for expressing and validating the cytotoxic function of mouse perforin in RBL cells

FIG. 3 shows a schematic representation of the murine stem cell plasmid vector (MSCV). cDNA encoding mouse perforin was inserted into the EcoRI and XhoI sites of the polylinker region. This biscistronic plasmid contains the amphotropic MSCV 5′ long LTR which drives the expression of the gene of interest, the GFP cDNA, and the IRES which permits translation of GFP and a second protein of interest from the one mRNA transcript. The autonomous expression of GFP enables the rapid selection of transduced cells expected to express the transgene of interest.

FIG. 4 shows a schematic representation of IgE-dependent cross-linking of effector RBL cells to EL-4 target cells. RBL cells were triggered to exocytose their granule content by cross-linking their surface Fcε receptor with TNP-labelled EL-4 target cells via an anti-TNP IgE antibody.

FIG. 5 illustrates the flow cytometry analysis of GFP expression levels in 293 T cells transfected with MSCV or MSCV-Pfp plasmid DNA. Either empty-MSCV vector (upper panel) or MSCV containing WT perforin cDNA (lower panel) were co-transfected with the amphotropic helper plasmid into 293T packaging cells, for the generation of high-titred viral supernatant. The solid blue line shows baseline fluorescence of 293 T cells transfected with the helper plasmid alone.

FIG. 6 shows the flow cytometric analysis of GFP expression in RBL cells following transduction with viral supernatants obtained from 293T packaging cells. A) RBL cells were transduced with viral supernatants encoding either MSCV vector, or MSCV containing the perforin cDNA and analysed 3 days later for GFP expression. The small number of cells (0.2 to 2.0%) expressing significant fluorescence above background (M1 gate) were isolated, expanded and gave rise to the populations in the lower panels. B) In comparison to untransduced RBL cells (solid line), RBL cells which were isolated based on the expression of high levels of the GFP transgene were expanded to yield a population in which more than 90% of the cells were expressing GFP.

FIG. 7 shows the expression of perforin in RBL cells by Western blotting. Whole cell lysates of untransduced, empty vector-transduced (MSCV) or perforin-transduced (MSCV-Pfp) RBL cells were probed with the rat anti-mouse perforin monoclonal antibody (mAb), P1-8. The labels at left indicate the migration of protein size markers.

FIG. 8 illustrates the flow cytometry analysis of surface labelling of RBL cells with anti-TNP IgE. A) RBL cells were labelled with the anti-TNP IgE antibody at a number of different dilutions (½, 1/20, 1/50, 1/100) to determine the optimal concentration for surface labelling. Binding was detected by incubating with a secondary biotin-conjugated-anti-mouse IgE antibody and then with Streptavidin PerCP, for analysis by flow cytometry. B) RBL cells were incubated with the anti-TNP IgE antibody at either 37° C. or 4° C. for 15 or 60 minutes to determine optimal conditions for maximal binding.

FIG. 9 shows the cytotoxic function of RBL cells expressing perforin on EL-4 target cells, as detected in a 4 hour ⁵¹Cr release assay. RBL cells reconstituted for perforin expression were labelled with an anti-TNP IgE antibody and conjugated to TNP-labelled EL-4 cells which were preloaded with ⁵¹Cr. For negative controls, the assay was performed either in the absence of the crosslinking IgE antibody or TNP. RBL cells transduced with empty MSCV vector were included as a basal measure of RBL toxicity. All cells were incubated at a range of effect 9 or -target cell ratios. The data points represent the mean value (+/−standard error) of assays run in triplicate.

FIG. 10 shows the expression of perforin in RBL populations transduced with MSCV-Pfp. Four independent 293T transfections, giving rise to high-titred viral supernatant were used to generate RBL cells expressing MSCV-Pfp. Cells isolated on the basis of high expression of the GFP transgene were analyzed for perforin protein expression by probing with the monoclonal anti-perforin antibody, P1-8. The membrane was also probed with an anti-tubulin antibody as an indicator of protein loading.

FIG. 11 shows the cytotoxic function of independent RBL cell lines expressing MSCV-Pfp as measured in a 4 hour ⁵¹Cr release assay. Four independent RBL populations expressing MSCV-Pfp were incubated with ⁵¹Cr-loaded EL-4 target cells at a range of effector-target ratios. Effector cells were triggered to degranulate by using an anti-TNP IgE antibody which recognizes surface TNP on the target cells. For the assay, RBL cells transduced with empty MSCV vector were included as a basal measure of RBL toxicity. The data points are the means of triplicate assays +/−standard errors. This assay is representative of six experiments.

FIG. 12 shows a schematic diagram of the mouse perforin protein. Two of the many missense mutations identified in FHL are shown. The cDNA for perforin molecules incorporating the amino acid substitutions of Patient 5 (P5=G429E) and Patient 6 (P6=P345L) were subcloned in the MSCV vector. Also shown are the putative amphipathic alpha helix, cysteine-rich EGF-like domain and C2 phosopholipid-binding domain. Numerals indicate the numbered residues of perforin, including the 21 amino acid leader sequence.

FIG. 13 shows the flow cytometry analysis of GFP expression levels in 293T cells transfected with P5-Pfp and P6-Pfp cDNA. MSCV DNA constructs encoding the P5-Pfp and P6-Pfp cDNAs were co-transfected with the amphotrophic helper plasmid into 293T packaging cells, for the generation of high-titred viral supernatant. The solid blue line indicates the basal fluorescence of 293T cells transfected with the helper plasmid alone.

FIG. 14 shows the expression of GFP in RBL cells transduced with viral supernatants obtained from 293T packaging cell transfections. RBL cells were transduced with MSCV viral supernatants encoding P5-Pfp and P6-Pfp cDNAs. Cells expressing high levels of the transgene were isolated and expanded to yield population shown in the solid green line. Basal fluorescence of untransduced parental RBL cells is shown in the filled purple profile.

FIG. 15 shows the expression of perforin in RBL cells, detected by Western blotting. RBL lysates transduced with WT-Pfp, P5-Pfp, P6-Pfp or empty MSCV vector were analysed for perforin expression by immunoblotting with a monoclonal anti-mouse perforin antibody. The membrane was re-probed for tubulin to ensure equal protein loading.

FIG. 16 shows a 4 hour ⁵¹Cr release cytotoxicity assay measuring function of RBL cells expressing WT or mutated perforin. The capacity of RBL cells expressing WT or mutated perforin (P5-Pfp or P6-Pfp) to kill TNP-labelled EL-4 cells was analysed in a 4 hour ⁵¹Cr release assay. RBL cells transduced with empty MSCV-vector were included in the assay as a negative control.

FIG. 17 shows the isolation of cytoplasmic granules from RBL cells. A) Granules were fractionated by density gradient fractionation of disrupted RBL cells that expressed WT-Pfp, P5-Pfp, P6-Pfp or empty vector. Gradient fractions were analysed for the presence of perforin by Western blotting using a monoclonal anti-perforin antibody, P1-8. B) shows the (3-hexosaminidase activity in gradient fractions shown in A).

FIG. 18 shows the immunohistochemical detection of perforin in RBL granules. RBL cells expressing empty vector (MSCV), WT-Pfp or mutated perforin (P5-pfp and P6-Pfp) were stained for their perforin content using the anti-perforin mAb, P1-8. The signal was detected using a biotinylated-secondary antibody, peroxidase labelled streptavidin and a substrate chromogen which results in brown coloured precipitate at the antigen site. Granules within all transduced RBL cells were also viewed under high magnification. A representative RBL cell expressing WT-Pfp shows typical staining observed under higher power. Staining is representative of five fields from experiments performed on three separate days.

FIG. 19 shows the lysis of Jurkat cells by granules isolated from RBL cells as assayed in a 4 hour ⁵¹Cr release assay. A) Jurkat cells were incubated with granules isolated from WT-Pfp and empty-MSCV transduced RBL cells. The assay used serial dilutions of the granules and was carried with or without the addition of EGTA. B) Jurkat cells were incubated with granules isolated form WT-Pfp RBL cells and compared to the function of granules isolated from P5-Pfp and P6-Pfp RBL cells. The data points are the means of triplicate assays +/−standard errors. The assays are representative of 3 such experiments.

FIG. 20 shows the lysis of red blood cells by granules isolated from RBL cells as detected by hemoglobin release. Granules isolated from RBL cells expressing WT-Pfp, P5-Pfp or P6-Pfp were incubated with red blood cells for 30 minutes and the hemoglobin release measured. The assay was also carried out in the presence of EGTA and with empty-MSCV transduced RBL granules

FIG. 21 shows the degranulation of RBL cells as detected by immunohistochemical staining for perforin. RBL cells transduced with WT-Pfp or mutated perforin (P5-Pfp and P6-Pfp) were labelled with anti-TNP IgE antibody and were incubated in the presence or absence of TNP-labelled EL-4 cells to stimulate the RBL cells to degranulate. All cells were then stained for their perforin content using the anti-perforin mAb, P1-8. The signal was detected using a biotinylated-secondary antibody, peroxidase-labelled streptavidin and a substrate chromogen which results in brown coloued precipitate at the antigen site. RBL cells transduced with empty MSCV were included as a negative control for perforin staining. Staining is representative of five fields from experiments performed on three separate days.

FIG. 22 shows reduced cytotoxic activity and truncation of T224W mouse perforin expressed in RBL cells. Perforin-dependent ⁵¹Cr release is shown from TNP-labeled Jurkat cells coincubated with transiently transfected, sorted RBL cells for 4 h in the presence of anti-TNP IgE. The data points are shown as the mean±SD of triplicate samples and are representative of three similar assays. The Western blot (right) shows truncation of T224W perforin expressed in two independent transfection experiments (T224W-1 and T224W-2) compared with WT and T224R perforin.

FIG. 23 shows T224W and G428E perforin localize differently in RBL cells. (A) Immunohistochemistry of perforin-expressing RBL cells demonstrated with anti-perforin antibody PI-8 and counterstained with eosin. (B) RBL cells either unlabeled or labeled with anti-TNP-IgE were stained as in (A), after degranulation was induced by transient incubation with TNP-labeled target cells (magnification, 400×).

FIG. 24 shows reduced cytotoxic activity but normal apparent molecular mass of G428E mouse perforin expressed in RBL cells. (A) Western blot showing perforin expression in stably transduced RBL cells compared with IL18/IL-21-activated mouse NK cells and empty vector-expressing cells (GFP). (B) Perforin-dependent 51Cr release from TNP-labeled Jurkat cells coincubated for 4 h in the presence of anti-TNP IgE with RBL cells stably expressing WT or G428E perforin. The data points are shown as the mean±SD of three independent experiments. The Western blot (right) shows that G428E comigrates with WT perforin. GFP is the empty vector control. (C) RBL cells stably overexpressing WT or G428E perforin or the empty vector (GFP) were lysed and fractionated on a Percoll density gradient. Fractions were then analyzed for their perforin content by Western blotting and their β-hexosaminidase activity.

FIG. 25 shows that the G428E mutation significantly reduces calcium-dependent membrane binding of soluble perforin. Equal quantities of recombinant WT and mutant

perforin were tested for their capacity to bind to sheep erythrocytes in the absence (−) or presence (+) of 1 mM CaCl₂. The total input of perforin in each case is shown as (C).

FIG. 26 shows the location of two common perforin polymorphisms, and missense mutations identified in HLH. The putative domains of perforin are indicated as boxes, and the numerals indicate the approximate amino acid boundaries for each domain, designating the first residue of the leader as residue 1. The N-terminus is a predicted to have lytic properties; two Low homology regions have no significant similarity to other mammalian protein domains; Amphipathic α-helix is homologous to regions of the complement membrane attack complex components C5b to C9; the EGF-like domain is structurally similar to ubiquitous EGF domains, primarily due to highly conserved cysteine residues; the C2 domain is the calcium-binding region responsible for membrane binding of perforin. The asterisked residues A91V and N252S refer to suspected perforin polymorphisms.

FIG. 27 shows reduced expression and partial loss of function of A91V and the co-inherited substitution R232S. The effect is shown of PRF1 mutations identified in fraternal twins inheriting A91V, R232H and doubly mutated A91V/R232H perforin. The top panel shows a Western blot of whole cell extracts from RBL cells expressing the respective mutated perforin, and sorted as described in the Materials and Methods. The graphs shows 4 hour cytotoxicity assays using transiently transfected and sorted RBL cells as effector cells and 51Cr labelled Jurkat cells as targets at the effector/target (E/T) ratios indicated. The data shown are the means±SE of 4-9 independent experiments. For clarity, a subset of the data (the lower E/T ratios) is shown again in the larger plot.

FIG. 28 shows normal expression and function of perforin with a serine substitution at residue 252. Western immunoblot showing the relative expression of D252S, D252N (as in human perforin) and D252E (as in flounder perforin) in transiently transfected RBL cells. The line graph (middle) shows the lytic activity of D252S perforin (equivalent to N252S in humans) in the ⁵¹Cr release cytotoxicity assay. The bar chart (bottom) compares the lytic capacity of perforin variants at position 252 grafted on to mouse perforin: D252E found in flounder and D252N in human perforin. The data shown are mean±SD, and are representative of three independent experiments.

FIG. 29 shows the analysis of missense mutations of PRF1 on the expression and activity of perforin. RBL cells were transfected to express perforin bearing each of the missense mutations listed, then FACS sorted and used in Western blot analysis and 51Cr release cytotoxicity assays. Unless indicated otherwise, each mutated perforin was tested in the RBL-based assay at least 3 times at E/T ratios of 30:1, 10:1 and 2:1, using Jurkat T lymphoma cells as targets. The mutations were classified according to the HLH patient's genotype: (A) those identified in homozygous patients (B) mutations identified in compound heterozygotes, where the second allele encoded a frame-shift or premature termination of the protein (C) mutations identified in compound heterozygotes with missense mutations in both alleles of PRF1. The Western immunoblots show the relative level of expression of mutated perforin in equivalent numbers of FACS-sorted RBL cells. The original reference for each patient is shown in the first column as a superscript. Age of HLH diagnosis is indicated in months, as described in the corresponding reference. Italics designate perforin mutations analysed previously by us elsewhere. Amino acid conservation is derived from the amino acid sequence alignment of mammalian and flounder perforins, as in PredictProtein (EMBL-Heidelberg).

FIG. 30 shows the effects of various substitutions at residue 232 of perforin on RBL-mediated cytotoxicity. ⁵¹Cr release cytotoxicity assays using transfected RBL cells and Jurkat target cells at the E/T ratios are indicated, comparing the cytotoxic function of R232C and R232H (substitutions identified in HLH patients) with WT and R232S (flounder) perforins.

FIG. 31 shows that V183G perforin has normal function, but the C279Y substitution results in loss of perforin function. ⁵¹Cr release cytotoxicity assays using transfected RBL cells and Jurkat target cells at the E/T ratios are indicated, comparing the putative perforin mutation V183G (top) and C279Y perforin (bottom) with WT perforin.

FIG. 32 shows inhibitor compound 46553 blocking the synergistic pro-apoptotic function of perforin and Granzyme B. Perforin was used at a 1:10,000 dilution to obtain 10-20% killing determined via perforin titration, as hereinbefore described. Granzyme B was used at 1 ug/ml.

Legend: P (perforin), I (inhibitor compound 46553), d or D (DMSO) and B (Granzyme B).

### DETAILED DESCRIPTION OF THE INVENTION

### Methods of Retroviral-Mediated Expression of Recombinant Perforin in a Cell

In an aspect of the present invention, there is provided a method of expressing perforin, or a fragment or variant thereof, in a cell, said method comprising transfecting a cell with a retroviral vector capable of driving the recombinant expression of said perforin, or a fragment or variant thereof, in the cell.

The invention particularly relates to expressing recombinant perforin using a retroviral system compared with standard methods of cellular expression such as by CaPO₄ precipitation, lipofectamine or similar agents or electroporation.

Throughout the description and claims of this specification the word “comprise”, and variations of the word such as “comprising” and “comprises”, are not intended to exclude other additives or components or integers or steps.

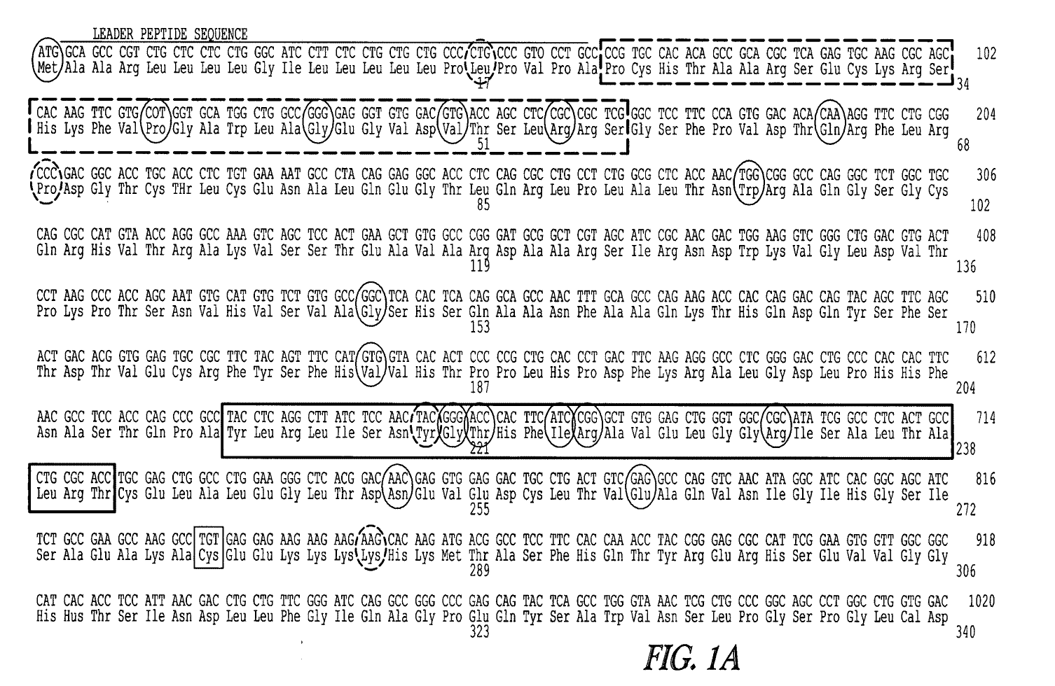

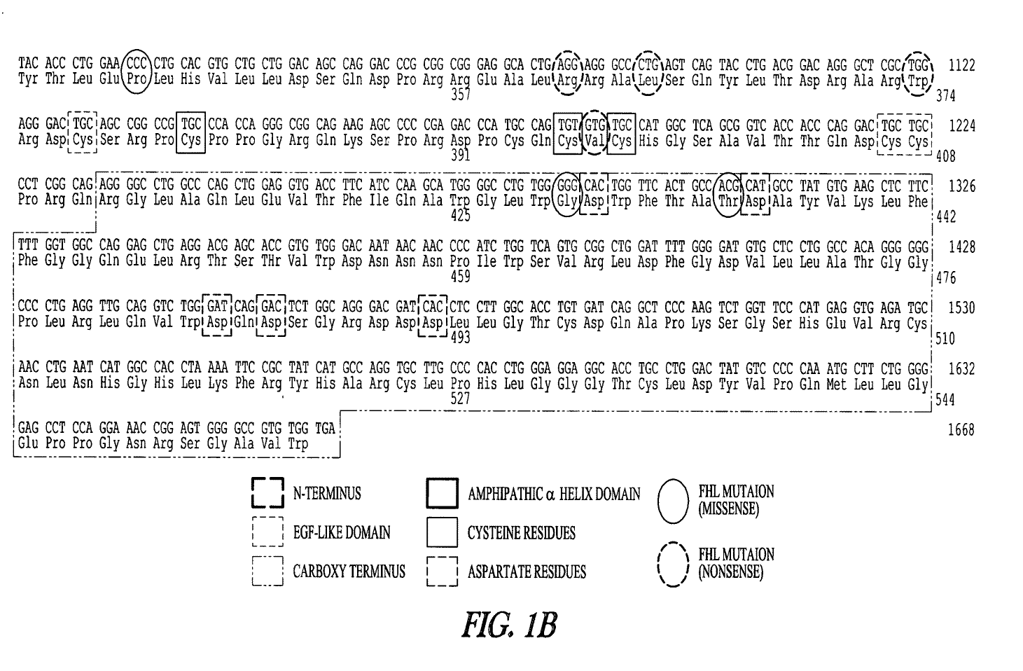

The terms “perforin”, “cytolysin”, “pore-forming protein (pfp)” and “C9-like protein” are used interchangeably herein and preferably encompass perforin polypeptides and fragments thereof in various forms, including naturally occurring or synthetic variants. Examples of perforins encompassed by the present invention include human perforin having an amino acid sequence as shown in FIG. 1. Also encompassed by the present invention are mouse and rat perforin isoforms, although perforins derived from other species, including those made by lower organisms such as bacteria, are also envisaged.

The perforin gene has been mapped to chromosome 10 in the mouse (Trapani et al., 1990, J Exp Med, 171:545-557) and chromosome 17 in humans (Shinkai et al., 1989, Immunogenetics, 30:452-457). It was found that exon 1 encodes an untranslated sequence, and the entire protein is encoded by a portion of exon 2 and all of exon 3, which also contains a 3′ untranslated region. The cloning of perforin cDNA encoding mouse (Kwon et al., 1989, Biochem Biophys Res Commun, 158:1-10; Lowrey et al., 1989, Proc Natl Acad Sci USA, 86:247-251), human (Lichtenheld and Podack, 1989, J Immunol, 143:4267-4274) and rat (Ishikawa et al., 1989, J Immunol, 143:3069-3073) perforin indicate that the human and mouse perforin are approximately 68% identical at the amino acid level, and that mouse and rat perforins are about 86% identical. Both human and mouse proteins are 534 amino acids in length, however the human leader peptide sequence (21 amino acids) is longer than the mouse counterpart by one residue. Perforin contains 20 cysteine residues, which are completely conserved across the three species, and these are believed to form 10 intra-chain disulphide bonds.

Early functional studies that noted the similarity of the pores formed by perforin and by the complement MAC (particularly C9) spurred search for structural and functional similarities between the two proteins. However, analysis of the primary sequence shows that the two proteins share only 20% homology in a stretch of 300 amino acids about the centre of the perforin molecule (Shinkai et al., 1988, Nature, 334:525-527) while the remainder shows no similarity at all. In this central portion are two regions of even higher homology. Residues 211-241 correspond to an area in the complement proteins, which display high amphipathic character. It been proposed that, upon attachment to the membrane, a marked conformational change occurs in the molecule, resulting in the exposure of this amphipathic alpha-helical region, enabling insertion into the lipid membrane. The second strongly conserved domain is the region between residues 376-409 which has similarity to the epidermal growth factor (EGF)-like repeat domains also found in the MAC proteins (Shinkai et al., 1988, Nature, 334:525-527). The six conserved cysteines present in this region may form intramolecular disulphide bonds, contributing towards maintaining a functionally important structure or may be the site of aggregation with other perforin monomers into a functional pore. The amino terminal 100 residues and the carboxy terminal 150 residues are completely unique to perforin. In a study carried out by Ojcius and colleagues (Ojcius et al., 1991, Proc Natl Acad Sci U S A, 88: 4621-4625), the use of a synthetic peptide corresponding to the 34 N-terminal residues, demonstrated that this region possessed strong membrane disrupting properties.

As used herein, the term “native” preferably refers to a perforin polypeptide molecule having an amino acid sequence that occurs in nature (e.g., a natural protein). Native perforin, or naturally occurring perforin, may be identified as one of the main constituents of cytocidal granules, is found to migrate with a molecular mass of approximately 66 kDa upon reduction and SDS-polyacrylamide gel electrophoresis, and migrates more slowly under non-reducing conditions (70-75 kDa), suggestive of a tightly disulphide-bonded structure in its native form. In the presence of calcium ions (Ca²⁺), perforin monomers aggregate into tubular structures that span the lipid bilayer, producing circular lesions (varying between 6 and 20 nm in diameter) that are thought to grow in diameter through the progressive recruitment of additional monomers.

Variants of perforin may exhibit amino acid sequences that are at least 80% identical to a native perforin polypeptide or fragment thereof. Also contemplated are embodiments in which a variant comprises an amino acid sequence that is at least 90% identical, preferably at least 95% identical, more preferably at least 98% identical, even more preferably at least 99% identical, or most preferably at least 99.9% identical to the native perforin polypeptide or fragment thereof. Percent identity may be determined by visual inspection and mathematical calculation. Among the naturally occurring variants and fragments thereof provided are variants of native perforin that retain native biological activity or a substantial equivalent thereof. Also provided herein are naturally occurring variants that have no substantial biological activity. These variants may also be derived from known HLH or FHL mutations, or may be empirical or deduced.

Variants of perforin preferably include polypeptides that are substantially homologous to the native form of perforin, but which have an amino acid sequence different from that of the native form because of one or more deletions, insertions or substitutions. Preferred embodiments include polypeptides that comprise from one to ten deletions, insertions or substitutions of amino acid residues when compared to a native sequence. A given sequence may be replaced, for example, by a residue having similar physiochemical characteristics. Examples of such conservative substitution of one aliphatic residue for another, such as Ile, Val, Leu or Ala for one another; substitution of one polar residue for another, such as between Lys and Arg, Glu and Asp, or Gln and Asn; or substitutions of one aromatic residue for another, such as Phe, Trp or Tyr for one another. Other conservative substitutions, e.g., involving substitutions of entire regions having similar hydrophobicity characteristics, are well known in the art. Variants may also be generated by the truncation of a native perforin polypeptide. Further variants encompassed by the present invention include, but are not limited to, deglycosylated perforin polypeptides, or fragments thereof, or those polypeptides demonstrating increased glycosylation when compared to native perforin. Also encompassed are perforin polypeptide variants with increased hydration. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue of a perforin polypeptide is preferably replaced with another amino acid residue from the same side chain family. In a preferred embodiment, mutations can be introduced randomly along all or part of a perforin coding sequence, such as by saturation mutagenesis, and the resultant mutants can be screened for perforin activity to identify variants that demonstrate the same, reduced or increased perforin activity in comparison to native perforin. Following mutagenesis, the encoded protein can be expressed recombinantly and the activity of the protein can be determined by the methods described herein.

Preferably, a variant of a perforin polypeptide will function as either an agonist (mimetic) or as an antagonist. An agonist of perforin can augment the activity of perforin or retain substantially the same, or a subset, of the biological activities of the naturally occurring form of perforin. An antagonist of perforin can inhibit one or more of the activities of the naturally occurring form of the polypeptide by, for example, competitively modulating perforin-mediated activity. Thus, specific biological effects can be elicited by treatment with a variant of limited function. Preferably, treatment of a subject with a variant having a subset of the biological activities of the naturally occurring form of perforin has fewer side effects in a subject relative to treatment with the naturally occurring form of the polypeptide.

As used herein, the terms “perforin activity”, “biological activity of perforin” and the like preferably refer to the cytolytic activity of a perforin polypeptide; that is, its ability to bind to a target cell membrane and polymerise into pore-like transmembrane channels leading to cell lysis. The activity also includes the capacity to synergise with other toxins such as granule toxins and other molecules to induce apoptosis. The target cell can be any cell that is capable of being lysed by native perforin.

The biological activity of perforin can be assessed by the skilled addressee by any number of means known in the art including, but not limited to, the measurement of target cell lysis, the delivery of granzyme B molecules into the target cell, the measurement of target cell membrane disruption (such as by changes in ion transport), the induction of apoptosis in the target cell, the modification of vesicular trafficking and the general assessment of target cell death. The target cell may be a red blood cell (RBC) and hence a common means of measuring perforin activity is by a RBC lysis test. It may also be any nucleated cell.

In a preferred embodiment, the variant is a mutation of the perforin gene. More preferably, the mutation is a perforin gene identified in individuals with HLH, more preferably (FHL).

HLH and more preferably the genetically linked FHL is a congenital disorder inherited as an autosomal recessive trait, belonging to a group of haemophagocytic lymphohistiocytosis syndromes, which are characterized clinically by fever, hepatosplenamegaly and pancytopaenia. In addition, neurological involvement commonly develops during the course of HLH or FHL, with manifestations that may include convulsions, cranial nerve palsies, ataxia and coma in the terminal stages (Haddad et al., 1997). Frequent abnormalities associated with the clinical symptoms include hypertriglyceridemia, hypofibrinagenemia, and elevated cytokine levels such as IL-1, IL-6, TNF and IFN-γ. Histologically, there is an excessive expansion of CD8+ T cells and macrophages, and their infiltration to several organs such as the spleen, liver, bone marrow (BM), lymph nodes and central nervous system. Evidence of ingestion of blood cells (especially erythrocytes) by histiocytes (ie, hemophagocytosis) in a variety of tissues (especially in bone marrow and liver) and release of inflammatory cytokines results in massive tissue necrosis, organ failure and ultimately death of the child. The combination of clinical (fever, splenomegaly), laboratory (cytopenia, hypertriglyceridemia and/or hypofibrinogenemia) and morpholgic (hemophagocytosis) features serve as the diagnostic criteria for the disorder, however the diagnosis is often made post-mortem, suggesting the diagnostic difficulties of the disease. At present, HLH and more preferably FHL is curable only with chemotherapy in combination with bone marrow transplantation. The aggressive and often debilitating nature of this treatment regime highlights the importance of identifying new and improved therapeutic strategies. The clinical picture in HLH or FHL is believed to result from the inability of cytolytic lymphocytes to clear an infecting pathogen, similar to the pathogenesis observed in perforin GKO mice infected with LCMV in which the increased expansion of virus-specific T cells and inability to downregulate the immune response are prominent features. It is thought that in the absence of perforin-dependent cytotoxic mechanisms, antigen-presenting cells (APC) continue to present activatory and proliferative signals to the non-functional lymphocytes. Although a single causative infectious agent for this infantile disease has not yet been defined, viral infections, especially of the herpes group (Epstein Barr-virus and cytomegalovirus), have been detected in patients suffering from FHL (Imashuku et al., 1999). Mutations in the coding region of perforin gene have been found to account for approximately 30% of HLH or FHL cases, but this does not discount the possibility that defects may also lie at the level of regulatory factors governing the expression or activation of perforin.

Preferred perforin mutations are given in Table 1, which lists some of the mutations identified in FHL to date and summarises the spectrum of non-sense, missense mutations and frameshift mutations that are predicted to affect the coding sequence and function of the protein. For example, a mutation at Trp374, which results in a premature stop codon, is by far the most frequently reported mutation. This residue is located within the cysteine-rich EGF domain and is conserved in the human, mouse and rat gene. A large number of the missense mutations are in residues that are conserved between all three species, suggestive that such residues are critical for function of the proteins. The location of the mutations occurring within specific domains of the perforin molecule is represented diagrammatically in FIG. 1. Of particular interest are the missense mutations that will prove invaluable in evaluating how these critical residues participate in perforin function.

TABLE 1
Perforin gene mutations identified in FHL

| | | | | Residue | | |
|---|---|---|---|---|---|---|
| | | | | conserved | | |
| Type | Sequence | Amino | Predicted | in: | | |
| of mutation | alteration | acid # | effect | Mouse | Rat | Domain |
| Missense | 3 G → A | 1 | Met → Leu | Yes | No | Leader |
| Deletion | 50 C del | 17 | Frameshift | No | No | Leader |
| Insertion | 50 T insert | 17 | Frameshift | No | No | N-terminus |
| Missense | 116 C → A | 39 | Pro → His | Yes | Yes | N-terminus |
| Missense | 133 G → A | 45 | Gly → Arg | Yes | Yes | N-terminus |
| Missense | 148 G → A | 50 | Val → Met | Yes | Yes | N-terminus |
| Missense | 160 C → T | 54 | Arg → Ser | Yes | Yes | N-terminus |
| Nonsense | 190 C → T | 64 | Gln → stop | Yes | No | — |
| Deletion | 207 C del | 69 | Frameshift | Yes | Yes | — |
| Missense | 283 T → C | 95 | Trp → Arg | Yes | Yes | — |
| Missense | 445 G → A | 149 | Gly → Ser | Yes | Yes | — |
| Missense | 836 G → A | 183 | Val → Gly | Yes | No | — |
| Nonsense | 657 C → A | 219 | Try → stop | Yes | Yes | Transmembrane |
| Missense | 658 G → A | 220 | Gly → Ser | Yes | Yes | Transmembrane |
| Missense | 662 C → T | 221 | Thr → Ile | Yes | Yes | Transmembrane |
| Missense | 671 T → A | 224 | Ile → Asp | Yes | Yes | Transmembrane |
| Missense | 673 C → T | 225 | Arg → Trp | No | No | Transmembrane |
| Missense | 694 C → T | 232 | Arg → Cys | Yes | Yes | — |
| Missense | 695 G → A | 232 | Arg → His | Yes | Yes | — |
| Missense | 755 A → G | 252 | Asn → Ser | No | No | — |
| Missense | 781 G → A | 261 | Glu → Lys | Yes | Yes | — |
| Missense | 836 G → A | 279 | Cys → Tyr | Yes | Yes | — |
| Deletion | 853-855 | 285 | Frameshift | No | No | |
| | AAG del | | | | | |
| Missense | 1034 C → T | 345 | Pro → Leu | Yes | Yes | |
| Deletion | 1083 G del | 361 | Frameshift | Yes | Yes | |
| Deletion | 1090-1091 | 364 | Frameshift | No | No | |
| | CT del | | | | | |
| Nonsense | 1122 G → A | 374 | Trp → stop | Yes | Yes | EGF-like |
| | | | | | | domain |
| Insertion | 1182 T | 394 | Frameshift | Yes | Yes | EGF-like |
| | insert | | and stop | | | domain |
| Missense | 1286 G → A | 429 | Gly → Glu | Yes | Yes | C2 domain |
| Missense | 1304 C T | 435 | Thr → Met | Yes | Yes | C2 domain |

Leader = signal peptide at the N-terminus of the molecule.
Transmembrane = putative amphipathic alpha helix domain.
C2 domain = C2 calcium-binding domain identified by molecular modelling by Uellner et al. (1997, Embo J, 16: 7287-7296).
Amino acid and nucleotide numbering includes the 21 amino acid leader peptide.

Perforin mutations and polymorphisms are also detailed in the Examples section below, and include A91V, N252S, R225W and G429E.

The catalogue of inactivating missense perforin mutations, now being compiled as a result of characterised HLH or FHL mutations, offers the possibility of unique insights into the molecular and cellular functions of perforin. Hypothetically, such defects in perforin function may occur at numerous levels, including mRNA instability, defective protein folding or processing, faulty trafficking to the cytolytic granules or defective release from the CTL. A second category of defects should map downstream of perforin's release from the CTL and involve functions such as calcium binding and attachment or insertion into the lipid bilayer, or cause defective trafficking of granzyme B.

The Applicant has mapped the nature of two perforin point mutations that result in single amino acid substitutions, ^{Gly}428^{Glu} and ^{Pro}344^{Leu}, or an equivalent position in a conserved perforin sequence and has for the first time arrived at the surprising discovery that both mutated variants are capable of release through granule exocytosis. It has been deduced that in the mouse sequence the mutation occur at the Gly 428 and at the Pro 344 whereas in the human sequence, the mutation occurs at Gly 429 and Pro 345. Similar point mutations may occur in the perforin sequence of other species at an equivalent Gly and/or Pro moiety in the perforin sequence. These findings imply that the depletion of perforin observed in the CTL of patients that possess these mutations is due to the dysregulated release of perforin during immune challenge.

Thus, in a preferred embodiment, the perforin polypeptide variant has reduced biological activity when compared to native perforin. In a further preferred embodiment, the perforin polypeptide variant comprises the missense mutation at a Gly and/or a Pro residue in a conserved perforin polypeptide sequence equivalent to G428 and/or P344 in a mouse perforin sequence or a Gly 429 and/or Pro345 in a human perforin sequence. In a further preferred embodiment, the perforin polypeptide variant comprises the missense mutation G428E and/or P344L, residues that have also been found by the Applicant to be conserved in both the mouse and rat perforin polypeptides. In yet a further preferred embodiment, the perforin polypeptide variant comprises the missense mutation G429E and/or P345L, residues that have also been found by the Applicant to be conserved in the human perforin polypeptide.

In a further preferred embodiment, the perforin variant is a fusion protein comprising a native perforin polypeptide, or a fragment thereof, and an additional domain attached thereto, wherein the additional domain can be either naturally occurring or synthetic. Preferably, fusion proteins of the present invention comprise a number of amino acids added to a perforin polypeptide, or a fragment or variant thereof, usually to the amino terminus of the recombinant perforin polypeptide. Such fusion proteins can serve a purpose selected from the group including, but not limited to: 1) increasing expression of a recombinant perforin polypeptide; increasing the solubility of a recombinant perforin polypeptide; and aiding in the purification of a recombinant perforin polypeptide by acting as a ligand in affinity purification. Often, a proteolytic cleavage site is introduced at the junction of the fusion moiety and the recombinant perforin polypeptide to enable separation of the recombinant perforin polypeptide from the fusion moiety subsequent to purification of the fusion protein. Such enzymes, and their cognate recognition sequences, include Factor Xa, thrombin and enterokinase. Typical fusion proteins may be produced by using fusion expression vectors known to those skilled in the art, such as pGEX, pMAL and pRIT5 which fuse glutathione S-transferase (GST), maltose E binding protein, or protein A, respectively, to the target recombinant polypeptide.

As used herein, the term “fragment” preferably refers to a portion of a perforin polypeptide, or a variant thereof. Such fragments preferably comprise at least 1 amino acid residue, more preferably at least 5 amino acid residues, even more preferably at least 10 amino acid residues, and still more preferably at least 20 amino acid residues of a native perforin polypeptide, or a variant thereof.

In a further preferred embodiment, a fragment of a perforin polypeptide may comprise an immunogenic or antigenic region. A fragment may therefore comprise a portion of a perforin polypeptide, or a variant thereof that is recognized (i.e., specifically bound) by an immunoglobulin.

In a further preferred embodiment, a fragment of a perforin polypeptide may consist of the biologically active C-terminal domain. Such fragments may generally be identified using techniques well known to those skilled in the art in identifying perforin activity, as hereinbefore described. Perforin polypeptide fragments may also be identified by screening fragments for their ability to react with perforin-specific antibodies and/or antisera. Antisera and antibodies are “perforin-specific” if they specifically bind to a perforin polypeptide or a variant or fragment thereof (i.e., they react with a perforin in an enzyme-linked immunosorbent assay [ELISA] or other immunoassay, and do not react detectably with unrelated polypeptides). Such antisera and antibodies may be prepared as described herein, and using well-known techniques (see, for example, Harlow and Lane, Antibodies: A Laboratory Manual, Cold Spring Harbor Laboratory, 1988).

A perforin molecule also encompasses naturally occurring or synthetic nucleic acid molecules whose nucleotide sequence encodes a perforin polypeptide, or a fragment or variant thereof, as hereinbefore described. The term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

As used herein, a “naturally-occurring” nucleic acid molecule preferably refers to an RNA or DNA molecule having a nucleotide sequence that occurs in nature (e.g., encodes a natural protein).

As used herein, the terms “gene” and “recombinant gene” preferably refer to nucleic acid molecules which include an open reading frame encoding a perforin polypeptide, and can further include non-coding regulatory sequences, and introns.

For example, the perforin nucleic acid molecule preferably comprises a nucleotide sequence which is at least about 60%, preferably at least 65%, more preferably at least 70%, even more preferably at least 75%, still more preferably at least 80%, still more preferably at least 85%, still more preferably at least' 90%, still more preferably at least 91%, still more preferably at least 92%, still more preferably at least 93%, still more preferably at least 94%, still more preferably at least 95%, still more preferably at least 96%, still more preferably at least 97%, even more preferably still at least 98%, most preferably at least 99% or more homologous to the nucleotide sequence shown in FIG. 1. In the case of a nucleic acid molecule that is longer than or equivalent in length to the reference sequence, e.g., FIG. 1, the comparison is made with the full length of the reference sequence. Where the isolated nucleic acid molecule is shorter than the reference sequence, e.g., shorter than that depicted in FIG. 1, the comparison is made to a segment of the reference sequence of the same length (excluding any loop required by the homology calculation). The perforin nucleic acid molecule may be derived from any species, including, but not limited to, human, rat, mouse, bird, horse, and lower organisms such as bacteria.

A cell may be transfected (or transduced) with a retroviral vector according to the present invention through any means known in those skilled in the art. Such means include, but are not limited to, electroporation, the use of liposomes, and CaPO₄ precipitation.

Retroviral Vector

The present invention exploits the use of a retroviral vector to “carry” a nucleic acid molecule which encodes the perforin, a fragment or variant thereof, to transfect the cell which ultimately expresses the perforin, a fragment or variant thereof. Thus, in yet a further aspect of the present invention, there is provided a retroviral vector that is capable of driving the expression of perforin, or a fragment or variant thereof, in a cell transfected with said vector.

As used herein, the term “retroviral vector” preferably refers to gene transfer vehicles that exploit features of the retrovirus replication cycle, for example, high infection efficiency and stable co-linear integration of the virally transmitted information in a target cell chromosome.

Retroviral vectors useful to the present invention may be derived from any number of retroviruses, including, but not limited to, Moloney Murine Leukemia Virus, murine stem cell virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In a preferred embodiment, the retroviral plasmid vector is the murine stem cell virus (MSCV) vector or derivatives thereof. More preferably, particularly when applied to the transfection of human or mouse primary cells, the retroviral plasmid vector is pLXSN (GenBank accession no. M28248).

The retroviral vector preferably includes one or more promoters. Suitable promoters which may be employed include, but are not limited to, the retroviral long terminal repeat (LTR); the SV40 promoter; and the human cytomegalovirus (CMV) promoter (as described in Miller of al., Biotechniques, Vol. 7, No. 9, 980-990 (1989)), or any other promoter (e.g., cellular promoters such as eukaryotic cellular promoters including, but not limited to, the histone, pol III, and β-actin promoters). Other viral promoters that may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the perforin polypeptide, or a fragment or variant thereof, is preferably placed under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the P-actin promoter; and human growth hormone promoters. The promoter also may be a native promoter that controls the genes encoding perforin, or a fragment or variant thereof.

In a further preferred embodiment, the retroviral vector of the present invention further comprises a suitable marker gene so that transduced cells can be readily selected (referred to herein as a “selectable marker”). Preferably, the selectable marker is a drug resistant gene that provides a transformed cell with antibiotic resistance, a reporter gene that provides a transformed cell with an enzyme activity for detection thereof, or an inert protein that may be detected in the transformed cell by methods known in the art. For example, the selectable marker may be green fluorescent protein that may be detected upon expression in a transformed cell by visualisation through light microscopy under ultra-violet light. In a further example, both N2/ZipTKNEO vector (TKNEO, 1991, Blood, 78:310-317) and PM5neo vector (1995, Exp. Hematol., 23:630-638) contain neomycin resistance genes (neomycin phosphotransferase) as their selectable marker. Accordingly, cells transfected with these vectors are recognized by their resistance to antibiotics (neomycin, G418, etc.) that are inactivated by the gene product.

Packaging Cell

In a preferred embodiment, the present invention includes a further step of transfecting the retroviral vector into a “packaging cell”. Thus, in yet a further aspect of the present invention, there is provided a packaging cell transfected with a retroviral vector capable of driving the recombinant expression of perforin, or a fragment or variant thereof, in a cell. Preferably, the packaging cell is capable of producing an infectious particle capable of further infecting a host cell to express a recombinant perforin.

Thus, in a further aspect of the present invention, there is provided a retrovirus particle carrying a retroviral vector that is capable of driving the expression of perforin, or a fragment or variant thereof, in a cell.

As used herein, the term “packaging cell” preferably refers to a cell that comprises those elements necessary for the production of infectious recombinant viruses by providing elements which are lacking in a recombinant viral vector. Typically, such packaging cells contain one or more expression cassettes which are capable of expressing viral structural proteins (such as gag, pol and env) but they do not contain a packaging signal (such as psi). Thus, a packaging cell can only form empty virion particles by itself. Within this general method, the retroviral vector is introduced into the packaging cell, thereby creating a “producer cell.” As a result, this producer cell manufactures virion particles containing the retroviral vector comprising a polynucleotide sequence encoding a perforin, or a fragment or variant thereof.

The use of a packaging cell can insure that replication competent viruses are not produced, which could otherwise create an uncontrolled infection within the host. Packaging cells express proteins that code for the virus's capsid (a protein coat that covers the nucleoprotein core or nucleic acid of a virus particle), and the genes encoding these proteins are at different sites within the packaging cell genome. This can prevent the relatively likely recombination event that would otherwise enable vector DNA to pick up the genes necessary to produce a replication-competent retrovirus. Preferably, the packaging cell line will produce retroviruses which are capable of infection, but which contain only RNA coding for perforin, or a fragment or variant thereof, its promoter, and LTR's which enable the proper expression of the perforin gene.

Packaging cells suitable for use with the above-described retroviral vector constructs may be readily prepared (see, for example, PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

Examples of packaging cells include, but are not limited to, PG13 (ATCC CRL-10686), PG13/LNc8 (ATCC CRL-10685), PA317 (ATCC CRL-9078), cell strains described in U.S. Pat. No. 5,278,056, GP+E-86 (ATCC CRL-9642), GP+envAm-12 (ATCC CRL-9641), 293T, PE501, PA317.psi.-2, .psi.-AM, PA12, T19-14X, VT-19-17-H2, .psi.CRE, .psi.CRIP, GP+E-86, GP+envAm12, and DNA cell lines as described in Miller, Human Gene Therapy, 1:5-14 (1990), which is incorporated herein by reference in its entirety. Preferably, the packaging cell line is derived from a HEK 293 cell. Even more preferably, the packaging cell is derived from a HEK 293 101 cell.

The retroviral vector may transduce the packaging cells through any means known in the art. Such means include, but are not limited to, electroporation, the use of liposomes, and CaPO₄ precipitation.

In preferred packaging and producer cells, the toxic envelope protein sequences, and nucleocapsid sequences are all stably integrated in the cell. However, one or more of these sequences could also exist in episomal form and gene expression could occur from the episome.

In a preferred embodiment, the packaging cell lines are second generation packaging cell lines. In another preferred embodiment, the packaging cell lines are third generation packaging cell lines.

Simple packaging cell lines, comprising a provirus in which the packaging signal has been deleted, have been found to lead to the rapid production of undesirable replication competent viruses through recombination. In order to improve safety, second generation cell lines have been produced wherein the 3′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection.

In split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsulate and provide a membrane protein for a high titre vector particle production. The packaging cell may be a cell cultured in vitro, such as a tissue culture cell line. Suitable cell lines include, but are not limited to, mammalian cells such as murine fibroblast derived cell lines or human cell lines. Preferably, the packaging cell line is a human cell line, such as, for example, HEK 293, HEK 293T, TE671 or HT1080.

Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

Preferably, the packaging cell line generates infectious retroviral vector particles (virions) that comprise a polynucleotide sequence encoding perforin, or a fragment or variant thereof, as hereinbefore described. Such retroviral vector particles may then be employed to transduce a host cell, either in vitro or in vivo, for the purposes of expressing the polynucleotide sequence encoding a perforin, or a fragment or variant thereof. Thus, in a further aspect of the present invention, there is provided a retrovirus particle carrying a retroviral vector that is capable of driving the expression of perforin, or a fragment or variant thereof, in a cell.

Host Cell

In a further aspect of the present invention, there is provided a host cell or cell line transfected with a retroviral vector capable of driving the recombinant expression of perforin, or a fragment or variant thereof, in the cell.

Preferably, the host cell or cell line is a eukaryotic cell or cell line of any species selected from the group including embryonic stem cells, embryonic carcinoma cells, hematopoietic stem cells, hepatocytes, fibroblasts, myoblasts, keratinocytes, endothelial cells, bronchial epithelial cells and immune cells. The host cell may also be of a lower organism such as bacteria. Preferably, the eukaryotic cell is an immune cell selected from the group including basophils, eosinophils, lymphocytes, neutrophils, monocytes and natural killer cells. More preferably, the immune cell is a basophil and even more preferably, the immune cell is a rat basophilic leukemia (RBL) cell.

Cell Compositions

In yet another aspect, the present invention provides a composition of cells transfected with a retroviral vector that is capable of driving the expression of a perforin molecule, a fragment or variant thereof, as herein described. A “composition of cells”, as used herein, preferably refers to an in vitro preparation of dispersed cells. In the case of cultured cells, it consists of a preparation of at least 10% and more preferably 50% of the subject's cells. Alternatively, the composition of cells may refer to biological tissue obtained from a subject (in vivo or ex vivo) into which the aforementioned retroviral expression vector has been administered. “Subject”, as used herein, preferably refers to a mammal, e.g., a human, or to a non-human animal, including, but not limited to, a horse, cow, goat, rat or mouse.

Isolated Recombinant Perforin and Fragments, Variants or Mutated Forms Thereof.

In a further aspect, the present invention provides a recombinant perforin molecule produced by the methods as herein described. In a preferred embodiment, the recombinant perforin molecule is an isolated or purified perforin molecule that is either a recombinant perforin polypeptide, or a fragment or variant thereof, as herein described, or a nucleic acid molecule encoding said perforin. It is a further preferred embodiment that the recombinant perforin molecule is isolated from the aforementioned composition of cells.

Preferably, an “isolated or purified” perforin molecule is substantially free of cellular material or other contaminating proteins from the cell or tissue source from which the protein is derived. The term “substantially free” preferably refers to a preparation of perforin polypeptide having less than about 30%, 20%, 10% and more preferably 5% (by dry weight) of a non-perforin molecule (also referred to herein as a “contaminating molecule”). The perforin polypeptide is also preferably substantially free of culture medium, i.e., culture medium represents less than about 20%, more preferably less than about 10%, and most preferably less than about 5% of the volume of the protein preparation.

The term “isolated or purified perforin molecule” is also a reference to a perforin nucleic acid molecule that is separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

Screening Assays

In yet a further aspect of the present invention, there is provided a method of screening for compounds that modulate perforin expression and/or activity, said method comprising the steps of:
obtaining a host cell transfected with a retroviral vector which drives the expression of recombinant perforin, or a fragment or variant thereof or obtaining a sample of perforin;
exposing said cell or perforin to a test compound; and
   determining whether said test compound binds to and/or modulates the expression and/or activity of said perforin.

Preferably, the screening assay comprises host cells that express a perforin molecule of the present invention. Such host cells are preferably derived from mammals, yeast, Drosophila or E. coli, as hereinbefore described. A cell expressing the perforin molecule (or a cellular fraction comprising the expressed perforin polypeptide) is then exposed to a test compound to observe binding to the perforin molecule, or modulation of perforin expression and/or activity.

In a further preferred embodiment, there is provided a method of screening for compounds that modulate perforin activity, said method comprising the steps of;
obtaining a host cell transfected with a retroviral vector which drives the expression of recombinant perforin, or a fragment or variant thereof or obtaining a sample of perforin;
exposing said host cell or perforin to a test compound and a target cell; and
determining whether said test compound modulates the activity of said perforin upon said target cell.

The target cell may either be directly exposed to the admixture of host cell and test compound. Alternatively, the target cell may be exposed to an admixture of test compound and the recombinant perforin produced by the host cell subsequent to the removal of the host cell from the admixture. The determination of the activity of the recombinant perforin need not require the continued presence of the host cell.

The screening assay may also use a sample of perforin preferably in isolated form to test the effect of test compounds on perforin. The compounds may either inhibit perforin activity by acting directly on the perforin molecule or it may block perforin at the target cell to prevent the perforin from acting. Either way, perforin is targeted so that its direct activities are not effective on the target cell.

The compounds identified by the screening assays preferably bind a perforin molecule, or a fragment or variant thereof, and activate (agonists) or inhibit (antagonists) the expression and/or activity of perforin. Preferably, the identified compound (e.g. natural or synthetic proteins or drugs) increases (agonist) and/or decreases (antagonist) the activity of a native perforin.

In an alternate aspect of the present invention, there is provided a method of screening for compounds that modulate perforin expression and/or activity, said method comprising the steps of:
obtaining a target cell capable of being lysed by perforin;
obtaining a sample of perforin;
exposing said cell or perforin to a test compound; and
determining whether said test compound modulates the target cell such that the activity of perforin on the target cell is modulated.

This alternate means of screening for compounds that affect the activity of perforin is directed to identifying those compounds that can modulate a target cell, a receptor on the target cell or an interacting molecule such as a ligand on the surface of the target cell to which perforin is targeted such that the cell is modified to be less responsive to perforin or becomes more responsive to perforin. This screening method identifies those compounds that do not alter perforin per se, but changes the target cell or receptor that perforin acts toward. The perforin may be provided as isolated perforin obtained by any means.

Preferably, it is provided as the recombinant perforin produced by the methods described herein.

As used herein, the terms “perforin activity”, “activity of perforin” and the like preferably refer to the cytolytic activity of a perforin polypeptide; that is, its ability to bind to a target cell membrane and polymerise into pore-like transmembrane channels leading to cell lysis. The target cell can be any cell that is capable of being lysed by native perforin. In a preferred embodiment, the compound identified by said screening assays activates or inhibits one or more perforin activities as hereinbefore described.

As used herein, the terms “expression of perforin”, “perforin expression” and the like preferably refer to the concentration of a polynucleotide that encodes perforin, or a fragment or variant thereof, or may refer to a concentration of the perforin polypeptide, or a fragment or variant thereof.

The activity of perforin can be assessed by the skilled addressee by any number of means known in the art including, but not limited to, the measurement of target cell lysis, the delivery of granzyme B molecules into the target cell, the measurement of target cell membrane disruption (such as by changes in ion transport), the induction of apoptosis in the target cell, the modification of vesicular trafficking and the general assessment of target cell death.

The expression of perforin may be assessed by the skilled addressee by any number of means known in the art including, but not limited to, the measurement of messenger RNA (mRNA) encoding perforin, preferably expressed by the host cell, such as by Northern blot analysis or quantitative reverse transcription-polymerase chain reaction (RT-PCR), as well as by the measurement of the perforin polypeptide in the host cell, such as by enzyme-linked immunosorbent assay (ELISA), radioimmunoassay (RIA), Western blot or by an indirect determination of perforin activity as hereinbefore described, such that the concentration of perforin in a biological sample is directly (but not necessarily linearly) proportional to the level of perforin activity.

In another aspect there is provided a compound identified by a screening assay that modulates perforin expression and/or activity. These compounds encompass numerous chemical classes, though typically they are organic molecules, preferably small organic compounds having a molecular weight of more than 50 and less than about 2,500 Daltons. Such compounds can comprise functional groups necessary for structural interaction with proteins, particularly hydrogen bonding, and typically include at least an amine, carbonyl, hydroxyl or carboxyl group, preferably at least two of the functional chemical groups. The compounds may also comprise cyclical carbon or heterocyclic structures and/or aromatic or polyaromatic structures substituted with one or more of the above functional groups. The compounds may also comprise biomolecules including, but not limited to, peptides, saccharides, fatty acids, steroids, purines, pyrimidines, derivatives, structural analogs, or combinations thereof. However, this invention is not limited to these compounds.

The compounds may include, but are not limited to 1) peptides such as soluble peptides, including Ig-tailed fusion peptides and members of random peptide libraries (see, e.g., Lam et al., 1991, Nature 354:82-84; Houghten et al., 1991, Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al., 1993, Cell 72:767-778); 3) antibodies (e.g., polyclonal, monoclonal, humanized, anti-idiotypic, chimeric, and single chain antibodies, as well as Fab, F(ab′)₂, Fab expression library fragments, and epitope-binding fragments of antibodies); and 4) small organic and inorganic molecules.

The compounds can be obtained from a wide variety of sources such as, but not limited to libraries of synthetic or natural compounds. Synthetic compound libraries may be commercially available from, for example, Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich Chemical Company, Inc. (Milwaukee, Wis.). Natural compound libraries comprising bacterial, fungal, plant or animal extracts are available from, for example, Pan Laboratories (Bothell, Wash.). In addition, numerous means are available for random and directed synthesis of a wide variety of organic compounds and biomolecules, including expression of randomized oligonucleotides.

Alternatively, libraries of natural compounds in the form of bacterial, fungal, plant and animal extracts may be produced. Methods for the synthesis of molecular libraries are readily available (see, e.g., DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233). In addition, natural or synthetic compound libraries and compounds can be readily modified through conventional chemical, physical and biochemical means (see, e.g., Blondelle et al., 1996, Trends in Biotech. 14:60), and may be used to produce combinatorial libraries. In another approach, previously identified pharmacological agents can be subjected to directed or random chemical modifications, such as acylation, alkylation, esterification, amidification, and the analogs can be screened for perforin-modulating activity.

Numerous methods for producing combinatorial libraries are known in the art, including those involving biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide or peptide libraries, while the other four approaches are applicable to polypeptide, peptide, non-peptide oligomer, or small molecule libraries of compounds (K. S. Lam, 1997, Anticancer Drug Des. 12:145).

Libraries may be screened in solution by methods generally known in the art for determining whether compounds will competitively bind at a common binding site. Such methods may including screening libraries in solution (e.g., Houghten, 1992, Biotechniques 13:412-421), or on beads (Lam, 1991, Nature 354:82-84), chips (Fodor, 1993, Nature 364:555-556), bacteria or spores (Ladner U.S. Pat. No. 5,223,409), plasmids (Cull et al., 1992, Proc. Natl. Acad. Sci. USA 89:1865-1869), or on phage (Scott and Smith, 1990, Science 249:386-390; Devlin, 1990, Science 249:404-406; Cwirla et al., 1990, Proc. Nat. Acad. Sci. USA 97:6378-6382; Felici, 1991, J. Mol. Biol. 222:301-310 and Ladner, U.S. Pat. No. 5,223,409).

A variety of other reagents may be included in the screening assay. These include reagents like salts, neutral proteins (e.g., albumin, detergents, etc.), which are used to facilitate optimal protein-protein binding and/or reduce non-specific or background interactions. Reagents that improve the efficiency of the assay, such as protease inhibitors, nuclease inhibitors, anti-microbial agents, etc., may be used. The components are added in any order that produces the requisite binding. Incubations are performed at any temperature that facilitates optimal activity, typically between 4° C. and 40° C. Incubation periods are preferably selected for optimum activity, but may also be optimized to facilitate rapid high-throughput screening. Normally, between 0.1 and 1 hours will be sufficient. Preferably, a plurality of assay mixtures is run in parallel with different test agent concentrations to obtain a differential response to these concentrations. Typically, one of these concentrations serves as a negative control, i.e., at zero concentration or below the level of detection.

The designing of mimetics to a known pharmaceutically active compound is also a known approach to the development of pharmaceuticals based on a “lead” compound. This might be desirable where the active compound is difficult or expensive to synthesize or where it is unsuitable for a particular method of administration, e.g., peptides are generally unsuitable active agents for oral compositions as they tend to be quickly degraded by proteases in the alimentary canal. Mimetic design, synthesis, and testing are generally used to avoid large-scale screening of molecules for a target property.

When designing a mimetic, it is desirable to firstly determine the particular regions of the compound that are critical and/or important in determining the target property. In the case of a peptide, this can be done by systematically varying the amino acid residues in the peptide (e.g., by substituting each residue in turn). These parts or residues constituting the active region of the compound are known as its “pharmacophore”.

Once the pharmacophore has been found, its structure is modelled according to its physical properties (e.g., stereochemistry, bonding, size, and/or charge), using data from a range of sources (e.g., spectroscopic techniques, X-ray diffraction data, and NMR). Computational analysis, similarity mapping (which models the charge and/or volume of a pharmacophore, rather than the bonding between atoms), and other techniques can be used in this modelling process.

In a variant of this approach, the three dimensional structure of the compound and its binding partner are modelled. This can be especially useful where the compound and/or binding partner change conformation on binding, allowing the model to take account of this in the design of the mimetic.

A template molecule is then selected, and chemical groups that mimic the pharmacophore can be grafted onto the template. The template molecule and the chemical groups grafted on to it can conveniently be selected so that the mimetic is easy to synthesize, is likely to be pharmacologically acceptable, does not degrade in vivo, and retains the biological activity of the lead compound. The mimetics found are then screened to ascertain the extent they exhibit the target property, or to what extent they inhibit it. Further optimization or modification can then be carried out to arrive at one or more final mimetics for in vivo or clinical testing.

Pharmaceutical Compositions

In yet another aspect of the present invention, there is provided a pharmaceutical composition comprising a recombinant perforin molecule as herein described, and/or an agonist or antagonist compound identified by the screening assays as herein described (also referred to herein as “active compounds”), together with a pharmaceutically acceptable carrier, excipient, diluent and/or adjuvant.

Pharmaceutical compositions of the present invention may be employed alone or in conjunction with other compounds, such as therapeutic compounds.

Such compositions typically include cells or biological tissue transfected with retroviral expression vectors capable of driving the expression of recombinant perforin, the perforin polypeptide, or a fragment or variant thereof, a nucleic acid molecule encoding said perforin, or a perforin-specific antibody, together with a pharmaceutically acceptable carrier, excipient, diluent and/or adjuvant. As used herein, the language “pharmaceutically acceptable carrier” preferably includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, or liquid polyetheylene glycol, and the like), and suitable mixtures thereof. The proper fluidity can be maintained, for example, by the use of a coating such as lecithin, by the maintenance of the required particle size in the case of a dispersion or by the use of surfactants. Prevention of the action of microorganisms can be achieved by incorporation of various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, or sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yield a powder of the active ingredient plus any additional desired ingredient from a previously sterile-filtered solution thereof.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavouring.

For administration by inhalation, the compounds are delivered in the form of an aerosol spray from a pressurised container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished with nasal sprays or suppositories. The compounds can be prepared in the form of suppositories (e.g., with conventional suppository bases such as cocoa butter and other glycerides) or retention enemas for rectal delivery.

For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. “Dosage unit form” as used herein preferably refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosages for use in humans. The dosage lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate perforin activity is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell, R. J. et al. (1996) Current Opinion in Biotechnology 7:89-94 and in Shea, K. J. (1994) Trends in Polymer Science 2:166-173. Such “imprinted” affinity matrices are amenable to ligand-binding assays, whereby the immobilized monoclonal antibody component is replaced by an appropriately imprinted matrix. An example of the use of such matrices in this way can be seen in Vlatakis, G. et al. (1993) Nature 361:645-647. Through the use of isotope-labeling, the “free” concentration of compound which modulates the expression or activity of perforin can be readily monitored and used in calculations of IC₅₀. Such “imprinted” affinity matrices can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz, D. et al. (1995) Analytical Chemistry 67:2142-2144.

As defined herein, a therapeutically effective amount of a recombinant perforin molecule (i.e., an effective dosage) preferably ranges from about 0.001 to 30 mg/kg body weight, more preferably about 0.01 to 25 mg/kg body weight, even more preferably about 0.1 to 20 mg/kg body weight, and still more preferably about 1 to 10 mg/kg, 2 to 9 mg/kg, 3 to 8 mg/kg, 4 to 7 mg/kg, or 5 to 6 mg/kg body weight. The composition can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including the activity of the specific compound employed, the age, body weight, general health, gender, and diet of the subject, the time of administration, the route of administration, the rate of excretion, any drug combination, the degree of expression or activity to be modulated. the severity of the disease or disorder, previous treatments and other diseases present.

For antibodies, the preferred dosage is generally 10 mg/kg to 20 mg/kg. However, if the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention as herein described can be inserted into vectors and used as gene therapy vectors. Preferably, the nucleic acid molecules are inserted into retroviral vectors, most preferably in the retroviral vector pLXSN. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is embedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment

In yet a further aspect of the present invention, there is provided a prophylactic or therapeutic method of treating a subject at risk of or susceptible to a disorder or having a disorder associated with undesired perforin expression and/or activity.

In a preferred embodiment, the prophylactic or therapeutic method comprises the steps of administering a therapeutic agent to a subject who has a disease, a symptom of disease or predisposition toward a disease associated with undesired perforin expression and/or activity as hereinbefore described, for the purpose to cure, heal alleviate, relieve, alter, remedy, ameliorate, improve, or affect the disease, the symptoms of the disease, or the predisposition towards the disease.

In a further preferred embodiment, the prophylactic or therapeutic method comprises the steps of administering a therapeutic agent to an isolated tissue or cell obtained from a subject who has a disease, a symptom of disease or predisposition toward a disease associated with undesired perforin expression and/or activity, as hereinbefore described, and reintroducing said tissue or cell into the subject for the purpose to cure, heal, alleviate, relieve, alter, remedy, ameliorate, improve, or affect the disease, the symptoms of the disease, or the predisposition towards the disease.

A “therapeutic agent” includes, but is not limited to, small molecules, peptides, antibodies, ribozymes, and antisense oligonucleotides, as herein described. With regards to both prophylactic and therapeutic methods of treatment, such treatments may be specifically tailored or modified, based on knowledge obtained from the field of pharmacogenomics. “Pharmacogenomics”, as used herein, preferably refers to the application of genomics technologies such as gene sequencing, statistical genetics, and gene expression analysis to drugs in clinical development and on the market. More preferably, the term refers to the study of how a patient's genes determine his or her response to a drug (e.g., a patient's “drug response phenotype”, or “drug response genotype”). Thus, another aspect of the present invention provides methods for tailoring an individual's prophylactic or therapeutic treatment with either the perforin molecules of the present invention or agents that modulate perforin expression and/or activity (such as those identified by screening assays as herein described), according to that individual's drug response genotype. Pharmacogenomics allows a clinician or physician to target prophylactic or therapeutic treatments to patients who will most benefit from the treatment and to avoid treatment of patients who will experience toxic drug-related side effects.

If the expression and/or activity of perforin are in excess, several therapeutic approaches are available. In one preferred approach, the therapeutic agent administered to a subject is an inhibitor compound (antagonist), as hereinbefore described, along with a pharmaceutically acceptable carrier, in an amount effective to inhibit perforin expression and/or activity, and thereby cure, heal alleviate, relieve, alter, remedy, ameliorate, improve, or affect the disease, the symptoms of the disease, or the predisposition towards the disease. For example, soluble forms of a perforin molecule capable of binding in competition with endogenous perforin may be administered. Preferred embodiments of such competitors comprise fragments of the perforin polypeptide that are able to bind native perforin to inhibit its biological activity, but have no inherent perforin activity of their own. A perforin antagonist may also include antibodies or antigen-binding fragments thereof (including, for example, polyclonal, monoclonal, humanized, anti-idiotypic, chimeric or single chain antibodies, and FAb, F(ab′)₂ and FAb expression library fragments, scFV molecules, and epitope-binding fragments thereof), oligonucleotides or perforin fragments or other small molecules that bind to a native perforin polypeptide and inhibit the biological activity of said native perforin.

The antagonist may also take the form of a compound that affects the target cell such that the target cell is modified and is no longer responsive to perforin or is less responsive to perforin. Here the treatment is not directed to perforin per se, but on the target cell. This allows for more accurate targeting of those cells that are targeted by perforin thereby protecting those cells from further attack.

Conditions in which perforin expression and/or activity is in excess, and where it is desirable to reduce said expression and/or activity, may be identified by those skilled in the art by any or a combination of diagnostic or prognostic assays known in the art. For example, a biological sample obtained from a subject (e.g. blood, serum, plasma, urine, saliva, and/or cells derived therefrom) may be analysed for perforin expression and/or activity as hereinbefore described. Such conditions include, but are not limited to, juvenile diabetes mellitus (type 1 or insulin dependent), graft-versus-host disease, chronic or acute allograft rejection and any other conditions associated with cytotoxic T lymphocyte- or natural killer cell-mediated immune pathology.

Thus, in a preferred embodiment, the prophylactic and therapeutic methods of treatment of the present invention are applicable to the treatment and/or prevention of immune mediated conditions such as, but not limited to juvenile diabetes mellitus (type 1 or insulin dependent), graft-versus-host disease, chronic or acute allograft rejection and conditions associated with cytotoxic T lymphocyte- or natural killer cell-mediated immune pathology.

For treating conditions in which it is desirable to increase perforin expression and/or activity, several approaches are also available. In a preferred approach, the therapeutic agent administered to a subject are the recombinant perforin polypeptides or compounds identified by the aforementioned screening assays, which activate endogenous perforin expression and/or activity, ie., an agonist as herein described above, in combination with a pharmaceutically acceptable carrier, to thereby cure, heal alleviate, relieve, alter, remedy, ameliorate, improve, or affect the disease, the symptoms of the disease, or the predisposition towards the disease. Preferred embodiments of such agonists include fragments of perforin polypeptides, and fragments and variants thereof, that are able to bind native perforin to increase its biological activity. A perforin agonist may also include antibodies or antigen-binding fragments thereof (including, for example, polyclonal, monoclonal, humanized, anti-idiotypic, chimeric or single chain antibodies, and FAb, F(ab′)₂ and FAb expression library fragments, scFV molecules, and epitope-binding fragments thereof) or other small molecules that bind to a native perforin polypeptide and increase the biological activity of said native perforin.

Conversely, as for the antagonist, the present invention also provides for compounds that are agonists that can modify the target cell such that the cell becomes more responsive to perforin. This assists in modifying those cells that may be targeted for elimination by perforin. Compounds employed in this method may be attached to an identifying moiety such as an antibody so that the moiety identifies those cells which require elimination.

An agonist is preferably employed for therapeutic and prophylactic purposes for conditions in which enhanced perforin activity is desirable, including, but not limited to, those associated with viral infection (such as the human immunodeficiency virus (HIV) and Hepatitis C), various cancer (such as lymphoma) and tuberculosis. Preferably, the agonist is employed for the treatment of conditions in which enhanced cytolytic T lymphocyte activity is desired. The agonists can also be employed for therapeutic and prophylactic purposes for conditions associated with perforin deficiency, such as HLH and more preferably FHL.

Thus, in a preferred embodiment, the prophylactic and therapeutic methods of treatment of the present invention are applicable to the treatment and/or prevention of viral infection (such as the human immunodeficiency virus (HIV) and Hepatitis C), various cancer (such as lymphoma), tuberculosis, conditions in which enhanced cytolytic T lymphocyte activity is generally desired, and conditions associated with perforin deficiency, such as HLH and more preferably FHL.

Alternatively, gene therapy may be employed to effect the endogenous expression of perforin by a cell in a subject in need of such therapy, including, but not limited to, rats, mice, dogs, cats, cows, horses, rabbits, monkeys, and most preferably, humans. For example, producer cells (as hereinbefore described) comprising a retroviral vector driving the expression of perforin, or a biologically active fragment thereof, may be administered to a subject for engineering cells in vivo to express the recombinant perforin polypeptide in vivo. For overview of gene therapy, see, for example, Chapter 20, Gene Therapy and other Molecular Genetic-based Therapeutic Approaches, (and references cited therein) in Human Molecular Genetics, Strachan T. and Read A. P., BIOS Scientific Publishers Ltd (1996).

Further, antisense and ribozyme molecules that inhibit expression of the target gene can also be used in accordance with the invention to reduce the level of target gene expression. Still further, triple helix molecules can be utilized in reducing the level of target gene expression.

As used herein, the term “antisense” preferably refers to a nucleotide sequence that is complementary to a nucleic acid encoding perforin, or a fragment or variant thereof, as hereinbefore described, e.g., complementary to the coding strand of the double-stranded cDNA molecule or complementary to the mRNA sequence ecoding perforin, or a fragment or variant thereof. The antisense nucleic acid is preferably complementary to an entire perforin coding strand, or to only a portion thereof. In a further embodiment, the antisense nucleic acid molecule is antisense to a “non-coding region” of the coding strand of a nucleotide sequence encoding perforin, or a fragment or variant thereof (e.g., the 5′ and 3′ untranslated regions).

An antisense nucleic acid can be designed such that it is complementary to the entire coding region of perforin, but more preferably is an oligonucleotide which is antisense to only a portion of the coding or non-coding region of perforin mRNA. For example, the antisense oligonucleotide can be complementary to the region surrounding the translation start site of perforin mRNA. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art.

For example, an antisense nucleic acid (e.g., an antisense oligonucleotide) can be chemically synthesized using naturally occurring nucleotides or variously modified nucleotides designed to increase the biological stability of the molecule or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest).

In a further embodiment of the present invention, perforin short interfering nucleic acid molecules (siRNA) that inhibit expression of the target gene can also be used in accordance with the invention to reduce the level of target gene expression.

The term “perforin short interfering nucleic acid”, “perforin siNA”, “perforin short interfering RNA”, “perforin siRNA”, “perforin short interfering nucleic acid molecule”, “perforin short interfering oligonucleotide molecule”, or “chemically-modified perforin short interfering nucleic acid molecule”, as used herein, preferably refers to any nucleic acid molecule capable of inhibiting or down-regulating perforin gene expression, for example by mediating RNA interference (“RNAi”) or gene silencing in a sequence-specific manner. Chemical modifications can also be applied to any siNA sequence of the present invention. For example, the siNA can be a double-stranded polynucleotide molecule comprising self-complementary sense and antisense regions, wherein the antisense region comprises nucleotide sequence that is complementary to a nucleotide sequence encoding perforin or a portion thereof and the sense region having a nucleotide sequence corresponding to a nucleotide sequence encoding perforin or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example, wherein the double stranded region is about 19 base pairs); the antisense strand comprises nucleotide sequence that is complementary to a nucleotide sequence encoding perforin or a portion thereof and the sense strand comprises nucleotide sequence corresponding a nucleotide sequence encoding perforin or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions, wherein the antisense region comprises nucleotide sequence that is complementary to a nucleotide sequence in a separate target nucleic acid molecule or a portion thereof and the sense region having a nucleotide sequence corresponding to a nucleotide sequence encoding perforin or a portion thereof. The siNA can be a circular single-stranded polynucleotide having two or more loop structures and a stem comprising self-complementary sense and antisense regions, wherein the antisense region comprises nucleotide sequence that is complementary to a nucleotide sequence encoding perforin or a portion thereof and the sense region having a nucleotide sequence corresponding to a nucleotide sequence encoding perforin or a portion thereof, and wherein the circular polynucleotide can be processed either in vivo or in vitro to generate an active siNA molecule capable of mediating RNAi. The siNA can also comprise a single stranded polynucleotide having a nucleotide sequence complementary to a nucleotide sequence encoding perforin or a portion thereof (for example, where such siNA molecule does not require the presence within the siNA molecule of a nucleotide sequence corresponding to a nucleotide sequence encoding perforin or a portion thereof), wherein the single stranded polynucleotide can further comprise a terminal phosphate group, such as a 5′-phosphate or a 5′,3′-diphosphate. In a preferred embodiment, the siNA molecule of the present invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In a further embodiment, the siNA molecule of the present invention comprises a nucleotide sequence that is complementary to a nucleotide sequence encoding perforin or a portion thereof. In another embodiment, the siNA molecule of the present invention interacts with a nucleotide sequence encoding perforin in a manner that causes inhibition of expression of the perforin gene. As used herein, siNA molecules need not be limited to those molecules containing only RNA, but further encompasses molecules comprising chemically-modified nucleotides or those in combination with non-nucleotides. In certain preferred embodiments, the siNA molecule of the present invention lacks 2′-hydroxy (2′-OH) containing nucleotides. Such siNA molecules that do not require the presence of ribonucleotides within the siNA molecule to support RNAi can, however, have an attached linker or linkers or other attached or associated groups, moieties, or chains containing one or more nucleotides with 2′-OH groups. Optionally, siNA molecules of the present invention can comprise ribonucleotides at about 5, 10, 20, 30, 40, or 50% of the nucleotide positions. The modified siNA molecules of the invention can also be referred to as short interfering modified oligonucleotides “siMON.” As used herein, the term siNA is preferably meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), translational silencing, and others. In addition, as used herein, the term RNAi is preferably meant to be equivalent to other terms used to describe sequence specific RNA interference, such as post transcriptional gene silencing, or epigenetics. For example, siNA molecules of the invention can be used to epigenetically silence genes at both the post-transcriptional level or the pre-transcriptional level. In a non-limiting example, epigenetic regulation of perforin gene expression by siNA molecules of the present invention can result from siNA-mediated modification of the chromatin structure to alter perforin gene expression.

The antisense and short interfering RNA molecules of the present invention are typically administered to a subject (e.g., by direct injection at a tissue site), or generated in situ such that they hybridise with or bind to cellular mRNA and/or genomic DNA encoding perforin to thereby inhibit expression of said perforin, e.g., by inhibiting transcription and/or translation. Alternatively, the molecules can be modified to target selected cells and then administered systemically. For systemic administration, antisense or siRNA molecules can be modified such that they specifically bind to receptors or antigens expressed on a selected cell surface, e.g., by linking the molecules to peptides or antibodies that bind to cell surface receptors or antigens. The molecules can also be delivered to cells using vectors, or by viral mechanisms (such as retroviral or adenoviral infection delivery). To achieve sufficient intracellular concentrations of the molecules, vector constructs in which the molecule is placed under the control of an appropriate promoter.

In yet another embodiment, the antisense nucleic acid molecule of the present invention is an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for perforin-encoding nucleic acid molecules can include one or more sequences complementary to the nucleotide sequence of perforin cDNA disclosed herein, and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a perforin-encoding mRNA (see, e.g., Cech et al. U.S. Pat. No. 4,987,071; and Cech et al. U.S. Pat. No. 5,116,742). Alternatively, perforin mRNA can be used to select a catalytic RNA having a specific ribonuclease activity from a pool of RNA molecules (see, e.g., Bartel, D. and Szostak, J. W. (1993) Science 261:1411-1418).

In a further embodiment, perforin expression can be inhibited by targeting nucleotide sequences complementary to the regulatory region of the perforin (e.g., a perforin promoter and/or enhancers) to form triple helical structures that prevent transcription of the perforin gene in target cells (see generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15). The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The antisense molecules may also be modified at the base moiety, sugar moiety or phosphate backbone to improve, e.g., the stability, hybridization, or solubility of the molecule. For example, the deoxyribose phosphate backbone of the nucleic acid molecule can be modified to generate peptide nucleic acids (see Hyrup B. et al. (1996) Bioorganic & Medicinal Chemistry 4 (1): 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93:14670-675.

PNAs of perforin nucleic acid molecules can be used in therapeutic and diagnostic applications. For example, PNAs can be used as antisense or antigene agents for sequence-specific modulation of gene expression by, for example, inducing transcription or translation arrest or inhibiting replication.

PNAs of perforin nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In other embodiments, the antisense molecules may comprise other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, antisense molecules can be modified with hybridization-triggered cleavage agents (See, e.g., Krol et al. (1988) BioTechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

It is possible that the use of antisense, siRNA, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy methods.

Another method by which nucleic acid molecules may be utilized in treating or preventing a disease characterized by undesired perforin expression and/or activity is through the use of aptamer molecules specific for perforin. Aptamers are nucleic acid molecules having a tertiary structure which permits them to specifically bind to protein ligands (see, e.g., Osborne, et al. (1997) Curr. Opin. Chem. Biol. 1(1):5-9; and Patel, D. J. (June 1997) Curr. Opin. Chem. Biol. 1(1):32-46). Since nucleic acid molecules may in many cases be more conveniently introduced into target cells than therapeutic protein molecules may be, aptamers offer a method by which perforin activity may be specifically decreased without the introduction of drugs or other molecules which may have pluripotent effects.

In conjunction with the treatment of diseases or conditions associated with undesired perforin expression and/or activity, pharmacogenomics (i.e., the study of the relationship between an individual's genotype and that individual's response to a foreign compound or drug) may also be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent to modulate perforin expression and/or activity, as well as tailoring the dosage and/or therapeutic regimen of such treatment.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms.

One pharmacogenomic approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high-resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., perforin), all common variants of that gene can be fairly easily identified in the population and it can be determined if having one version of the gene versus another is associated with a particular drug response.

Alternatively, a method termed the “gene expression profiling” can be utilized to identify genes that predict drug response. For example, the gene expression of an animal dosed with a drug (e.g., a perforin molecule or a modulator of perforin expression according to the present invention) can give an indication whether gene pathways related to toxicity have been turned on.

Information generated from more than one of the above pharmacogenomic approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a therapeutic agent as hereinbefore described.

Monitoring the influence of agents (e.g., drugs) on the expression and/or activity of perforin can be applied in clinical trials. For example, the effectiveness of a compound, identified by a screening assay as described herein, to increase perforin expression and/or activity can be monitored in clinical trials of subjects exhibiting decreased perforin expression and/or activity. Alternatively, the effectiveness of an agent determined by a screening assay to decrease perforin expression and/or activity can be monitored in clinical trials of subjects exhibiting increased perforin expression and/or activity. In such clinical trials, the expression and/or activity of perforin, and preferably, other genes that have been implicated in, for example, conditions associated with undesired perforin expression and/or activity (i.e. surrogate markers) can be used as a “read out” or markers of the phenotype of a particular cell.

It would also be well appreciated by one skilled in the art that the methods of treatment hereinbefore described could be used in any number of combinations with each other, or with other treatment regimes currently employed in the art.

Examples of the procedures used in the present invention will now be more fully described. It should be understood, however, that the following description is illustrative only and should not be taken in any way as a restriction on the generality of the invention described above.

### EXAMPLES

### Example 1

### The Expression of Wild Type Mouse Perforin in RBL Cells

A. Expression of Perforin

The following description includes materials and methods used for the recombinant expression, analysis and assesment of wild type mouse perforin.

i) Cell Culture

The cell lines RBL-2H3 (American Type Culture Collection-ATCC), 293T (human embryonic kidney) and EL-4 (mouse thymoma) were maintained in Dulbecco's modified Eagle's (DME) medium supplemented with 10% fetal calf serum (FCS), 2 mM glutamine (Commonwealth Serum Laboratories, Parkville, Melbourne, Australia (CSL)) and 100 μg/ml each of streptomycin and penicillin (Gibco, Grand Island, N.Y.). The cell lines were maintained in a humidified incubator at 37° C. in 10% CO₂. For harvesting RBL-2H3 and 293T cells, cells were washed once in PBS, and a trypsin-EDTA solution (CSL, Australia) was added to detach cells from the tissue culture flask. Cells were washed once in PBS before use.

(ii) Generation of a Plasmid Vector Encoding Wild Type Mouse Perforin

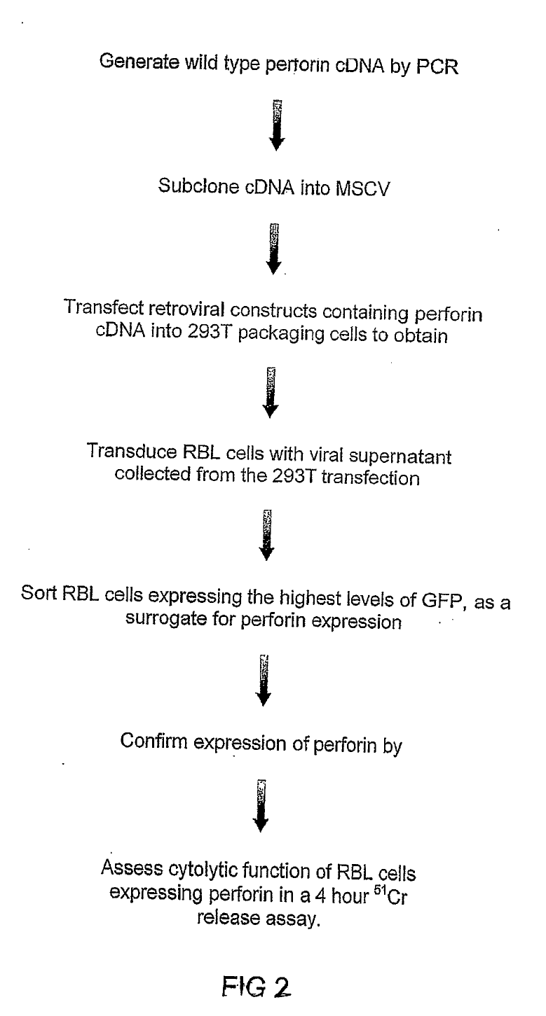

The overall strategy followed the expression of wild type mouse perforin (Pfp) in RBL cells using retroviral transduction is depicted in FIG. 2. This represents in a summarized form the protocols outlined below.

(iii) Subcloning of Pfp cDNA into MSCV

For the subcloning of Pfp cDNA into the murine stem cell retroviral vector, MSCV (kindly provided by Prof. Steve Jane, Royal Melbourne Hospital, Melbourne), a 1.8 kb fragment of DNA was amplified by polymerase chain reaction (PCR) using oligonucloeotides incorporating EcoRI and XhoI sites at their 5′ (Geneworks, Australia).

| | |
|---|---|
| Sense: | |
| 5′ CTCGAATTCGCATCATGGCCACGTGC 3′ | (SEQ ID NO: 1) |
| Antisense: | |
| 5′ CTATCTCGAGTTACCACACAGCCCCACTG 3′ | (SEQ ID NO: 2) |

The template DNA used in the reaction was a pEF-PGKpuroA construct containing the perforin cDNA previously amplified from RNA from the mouse Lymphokine activated killer (LAK) cell line, IMS-II using RT-PCR. The PCR was set up in a 500 volume and contained: 5 ng template DNA, 2.5 units Pfu Polymerase (Promega, NSW, Australia), 50 uM dNTP mix and 12.5 pmol of each primer in Pfu Polymerase buffer. The reaction was performed in a PTC-200 Peltier Thermal cycler (MJ Research Inc. Massachusetts) and consisted of the following cycles: 1 cycle at 94° C. for 2 min (denaturation); then 25 cycles at 94° C. for 30 secs, 60° C. for 30 secs (annealing), 72° C. for 4.5 mins (synthesis), and finally 1 cycle at 72° C. for 7 mins. The PCR products were separated by electrophoresis on a 1% (w/v) agarose/TBE (89 mM Tris-borate pH 8.0, 89 mM boric acid, 2 mM EDTA) gel and visualised by the addition of 1 μl ethidium bromide (10 mg/ml). The DNA bands were excised from the gel and purified using the Jetsorb DNA Gel extraction kit (Genomed, Inc. USA) according to the manufacturer's instructions.

The purified PCR products were prepared for subcloning by digestion with EcoRI and XhoI (Promega, NSW, Australia). Reactions contained EcoRI and XhoI (1 U of each), 100 ng DNA, restriction enzyme buffer appropriate for the respective enzyme, and were incubated overnight at 37° C. The digested cDNA (60 μg) was ligated with the MSCV DNA (50 ng) previously digested with EcoRI and XhoI using T4 DNA Ligase (Promega, Australia) (1 U) in ligase buffer at 14° C. overnight.

(iv) Transformation of Competent Bacteria

E. Coli strain Top 10F bacteria (100 μl) were mixed with 4 μl MSCV-Pfp ligation mix (above) and incubated on ice for 30 minutes. The bacteria were heated to 42° C. for 45 seconds, and then left at room temperature for 5 mins. Luria-Bertani broth (LB-broth) (900 μl) was added and the mix was cultured with shaking at 37° C. for one hour. The transformation reaction was then plated onto LB-media plates supplemented with ampicillin and tetracycline (10 μg/mL of each) and cultured at 37° C. overnight. Transformants were picked at random and cultured in 2 ml LB-broth supplemented with 10 μg/mL Ampicillin and Tetracycline overnight at 37° C. for further analaysis.

(v) Small Scale Preparation of Plasmid DNA

Plasmid DNA from the overnight cultures was isolated from the bacterial cells using a miniprep plasmid purification kit (Mo Bio Lab Inc. USA) as per the manufacturer's instructions. Plasmid identity was confirmed by restriction enzyme digestion with EcoRI and XhoI and agarose gel electrophoresis. Miniprep clones containing the cDNA insert were sequenced in full to verify that no PCR-related mutations were introduced. Sequencing was carried out using the automated Big Dye Terminator reaction protocol (as per manufacturer's instructions) and analysed at the Automated DNA Analysis Facility of The University of New South Wales (Australia).

(vi) Large Scale Preparation of Plasmid DNA

Large scale preparation of the sequenced Pfp DNA was obtained by the ‘alkaline lysis’ method and purified on a CsCl-ethidium bromide gradient. Bacteria from a 500 ml overnight culture in LB-broth supplemented with Ampicillin and Tetracycline (50 μg/ml) were pelleted by centrifugation for 10 minutes at 4000 rpm in a RC-C Sorval centrifuge (Sorval Instruments, Du Pont). The pellet was resuspended in 10 ml solution 1 (50 mM glucose, 25 mM TrisCl, pH 8, 10 mM EDTA, pH 8), lysed for 10 minutes on ice in 20 ml solution 2 (0.2 M NaOH, 1% SDS) and the pH was neutralized in 15 ml solution 3 (3M potassium acetate, pH 4.8) on ice for 10 minutes. The cellular debris was removed by centrifugation at 4000 rpm for 10 mins and the supernatant containing the plasmid DNA was filtered through cheesecloth. The DNA was precipitated by adding 35 ml isopropanol for 15 minutes on ice and pelleted at 12,000 rpm for 15 minutes at 4° C. The pellet was washed in 70%, then absolute ethanol, dried at room temperature and resuspended in 10 ml dd H₂O. The CsCl gradients were set up by adding 10.7 g CsCl and 500 μl Ethidium bromide solution (10 mg/ml) to the plasmid DNA. Following a 5 min centrifugation at 3200 rpm to pellet debris, the samples were loaded into Beckman Polyllomor Bell-top Quick-seal centrifuge tubes (Beckman Instruments Inc., CA, USA) and centrifuged in a Beckman TL-100 Ultracentrifuge (Beckman Instruments Inc.) overnight at 55,000 rpm, 20° C. The DNA band was recovered with a 26-gauge needle and the ethidium bromide was extracted by washing the sample three times with equal volumes of isoamyl alcohol and precipitated with 2.5 volumes of absolute ethanol at 4° C. overnight. The samples were pelleted at 13,000 rpm for 15 minutes at 4° C., washed in 70% ethanol, dried and resuspended in TE buffer pH 7.4. DNA was quantified both by visualisation on a 1% agarose gel, and measuring the absorbance at 260 nm.

(vii) Expression of Pfp Using a Retroviral Expression System

Expression of perforin using the retroviral expression system exploited several features of the MSCV vector (FIG. 3). The biscistronic plasmid contains several features which enable the selection of transduced cells: 1) the amphotropic MSCV 5′ long terminal repeat (LTR); 2) the cDNA for Green Fluorescent Protein (GFP); 3) the encephalomyocarditis internal ribosomal entry site (IRES) and 4) a bacterial origin of replication and the ampicillin resistance gene. The IRES allows for two genes to be transcribed on the same strand of mRNA, so that a marker can be placed downstream from the main gene to be transcribed and the two will be translated separately. The expression of GFP, which causes the cells to fluoresce under ultraviolet light, serves as a surrogate marker for perforin and enables the selection of cells expressing high levels of the transgene.

(viii) Generation of Recombinant Virus for Pfp Expression

MSCV DNA encoding the perforin gene (MSCV-Pfp) was transiently transfected into the 293T packaging cell line, which secretes viral particles into the culture supernatant used later to infect the RBL cells. The co-transfection of an amphotropic helper plasmid provides the retroviral DNA with viral envelope proteins recognized by the amphotropic receptor on a large number of mammalian cells, thereby facilitating the delivery of the foreign genes. Initially, 293T cells (5×10⁵) were plated in 100 mm Petri dishes overnight and 3 hours prior to transfection, the culture medium was replaced with fresh complete DMEM. The cells were transfected with MSCV vector DNA or MSCV-Pfp DNA by the calcium phosphate precipitation method (Gibco) according to the manufacturer's instructions. On the day of transfection, a DNA-CaCl₂ solution was prepared by mixing 25 μl M CaCl₂, 10 μg plasmid DNA (in 10 mM Tris-Cl, pH 7.5), 10 μg of helper plasmid cDNA encoding the gag and pol genes and water to a 200 μl final volume. Precipitation buffer was also prepared consisting of 100 μl of 500 mM HEPES-NaOH (pH 7.1), 125 μl of 2 M NaCl, 10 μl of 150 mM NaHPO₄—NaH2 PO₄ (pH 7.0) and water to a final volume of 1 ml. To 200 μl of the precipitation buffer, 200 μl of the DNA-CaCl₂ solution was added drop wise and the mixture constantly agitated. The mixture was kept at room temperature for 30 minutes and the resultant fine precipitate was added to a dish of 293T packaging cells. Cells were exposed to the DNA precipitate for 24 hours then the medium was replaced with fresh complete DMEM. After 48 hours, cells were harvested and analyzed for expression of GFP. GFP expression as measured by flow cytometry using FACScan (Becton Dickinson, San Hose, Calif.) determined the transfection efficiency, which is indicative of virus titre in the supernatant. Culture supernatant from the most efficient transfections (>30% GFP-expressing cells) were collected and stored in 1.5 ml aliquots for the transduction of RBL cells (see below).

(ix) Transduction of RBL Cells with Virus-Enriched Supernatant

For transduction using retrovirus, RBL cells were plated into 6-well plates at (2×10⁵ in 1 ml of complete DME-M). Cells were mixed with retroviral supernatant six times at 12 hourly intervals in the presence of 4 μg/ml polybrene, allowed to recover for 72 hours, then analysed for GFP expression by flow cytometry on a FACStar cell sorter. Cells in the population with the greatest GFP expression (up to 5% of cells) were selected for further expansion and screened for perforin expression and for functional analysis.

B. Analysis of Pfp Expression

(i) Preparation of Cell Lysates

Lysates of RBL cells transduced with MSCV-Pfp were analysed by western blotting to screen for protein expression. Cells were harvested and resuspended (2×10⁷/ml) in NP-40 lysis buffer (25 mM Hepes buffer, pH 7, 0.25 mM NaCl, 2.5 mM EDTA, 0.1% Nonidet-P40 (NP-40), 0.5 mM DTT, and a cocktail of protease inhibitors (Roche, Germany). Cells were incubated on ice for 20 min, then pelleted at 13000 rpm to remove cell debris. The collected supernatant was diluted in an equal volume of 2× sample buffer containing reducing agent (1.52 g Tris base, 20 ml glycerol, 2 g SDS, 2 ml 2-mercaptoethanol, 1 mg bromophenol blue, pH 6.8 up to 100 ml with H₂O), boiled at 95° C. for 5 mins and loaded onto a 10% (w/v) sodium dodecyl sulphate (SDS)-polyacrylamide gel.

(ii) SDS-Polyacrylamide Gel Electrophoresis and Immunodetection of Proteins

Polyacrylamide gels were assembled according to the Mini-Protean II Electrophoresis Cell (Bio-Rad, USA) specifications. Protein samples were resolved through 4.5% stacking gel (0.8 ml 30% Acrylamide/bis, 2.95 ml ddH2O, 1.25 stacking buffer, 50 μl 10% APS, 10 μl TEMED) and a 10% separation gel (2.75 ml 30% Acrylamide/bis, 3.25 ml ddH₂O, 2 ml separation buffer, 50 μl 10% APS, 10 ul TEMED. Electrophoresis was at 160 V in running buffer (0.1% SDS, 25 mM Tris-HCl, 192 mM glycine). Proteins separated by SDS-PAGE were transferred to nitrocellulose ‘Immobilon’ membrane (Millipore Bedford, Mass.) in western transfer buffer (48 mM Tris, 39 mM glycine, 20% methanol, pH 9.2) using the Trans-Blot SD Semi Dry Transfer Cell (Bio-Rad, Hercules, Calif., USA). Transfer was performed at 14 V, 0.5 A for 30 minutes. Non-specific binding of proteins to the membrane was blocked for 1 hour in a solution of 5% skim milk powder/PBS and then probed with a primary rat anti-mouse perforin antibody, PI-8 (stock concentration 1.7 mg/ml; kindly provided by Dr H. Yagita, Juntendo University School of Medicine, Tokyo, Japan), which was diluted 1/1000 in 5% skim milk buffer. The membrane was washed (3×8 mins) in 0.05%/Tween PBS, and the bound rat Ig was detected with a secondary goat anti-rat antibody conjugated to horse-radish peroxidase (HRP) ( 1/10000 dilution) for 1 hour at room temperature. The membrane was washed as before and the bound antibody visualised using the Enhanced Chemiluminescence (ECL) Detection System (Amersham International, UK) and exposure to X-OMAT AR Imaging film (Eastman Kodack company, Rochester, N.Y., USA). The membrane used in this western blot was also probed with an anti-tubulin antibody (Sigma) ( 1/3000) to ensure equal protein loading.

(iii) Labelling of RBL Cells with Anti-TNP IgE Antibody: Optimisation of Labelling Conditions

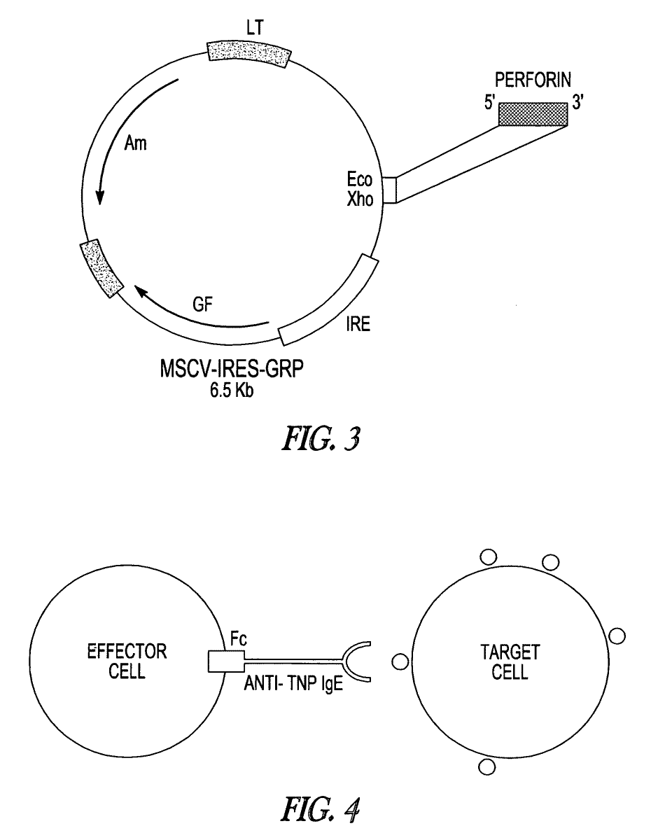

The ability of RBL cells expressing MSCV-Pfp to kill mouse thymoma EL-4 target cells was assessed in a 4 hour ⁵¹Cr release assay as herein below described (see section 2.5). The assay (previously described by Shiver et al, 1991) involved the use of an anti-trinitrophenyl (TNP) IgE antibody which crosslinks the Fcε receptor on RBL cells with TNP-labelled target cells and to stimulate granule secretion (FIG. 4). To determine the optimal concentration for anti-TNP IgE binding, RBL cells were labelled under various conditions and surface binding detected by flow cytometry. 1×10⁶ cells were labeled with varying dilutions of the hybridoma culture supernatant (kindly provided by Prof M. Hogarth, Austin Research Institute, Melbourne, Australia) containing anti-TNP IgE antibody (stock concentration 2 μg/ml). Antibody dilutions of ½, 1/10, 1/50 or 1/100 were set up in PBS and incubated for 1 hour at 37° C. Cells were washed three times and then incubated with a biotin-conjugated anti-mouse IgE antibody (PharMingen) at 1.25 μg/ml for a one hour. Cells were washed three times before the addition of Streptavidin-PerCP at 0.5 μg/ml for analysis by flow cytometry. To determine the optimal conditions for antibody binding, cells were incubated in a ½ dilution of the anti-TNP IgE hybridoma supernatant and incubated at 37° C. for 15 or 60 minutes and at 4° C. for 15 or 60 minutes. For detection of surface labeling, cells were incubated as mentioned above and analysed by flow cytometry.

C. Assessment of Pfp Cytolytic Function

(i) Dual Labeling of EL-4 Target Cells with ⁵¹Cr and TNP.

The EL-4 cells were loaded with ⁵¹Cr and labelled with the TNP hapten for use as target cells in the cellular cytotoxicity assay outlined as follows. Cells were washed twice in plain DME medium and resuspended in 100 μl of the same medium. The cells were labelled with 100 μCi ⁵¹Cr for 1 hr at 37° C. and washed three times with plain medium to remove free ⁵¹Cr. The cells were then labelled with TNP by resuspending them at 5×10⁶/ml in 1 mM TNBS (Fluka) solution (pH 7.4) for 15 mins at 37° C. Cells were washed three times in PBS and suspended at 1×10⁶/ml in 1% bovine serum albumin (BSA)/DME medium for use in the cytotoxicity assay.

(ii) Labelling of RBL Cells with IgE Antibody

RBL cells were labelled with the anti-TNP IgE antibody by resuspending at 5×10⁶ cells/ml in PBS containing antibody. Cells were incubated for 1 hour at 37° C., then washed three times in PBS and resuspended at 1×10⁷/ml in DME-M containing 1% BSA (CSL, Australia) for use in the cytotoxicity assay.

(iii) ⁵¹Chromium Release Cytotoxicity Assay

Cell death was assessed in ⁵¹Cr release assays by mixing IgE-labelled effector cells and ⁵¹Cr/TNP labelled target cells in 200 μl medium containing 1% BSA at a range of effector:target ratios. Experiments were carried out in 96 well V-bottom microtitre plates. The spontaneous release of ⁵¹Cr was determined by incubating the target cells with medium alone and the maximum release by adding HCl to a final concentration of 1M. As negative controls, EGTA was added to the reaction (final 2 mM). After 4 hours, the plates were centrifuged at 1500 rpm, 100 ul of supernatant was harvested and the released radioactivity measured by a Wizard 1470 Gamma counter.

Cytotoxicity was expressed as the percentage specific ⁵¹Cr release after subtracting spontaneous release. The percent specific lysis was calculated as follows: 100×[(experimental release−spontaneous release)/(maximum release−spontaneous release)].

A. Expression of Perforin in RBL Cells Using Retro Viral Expression System

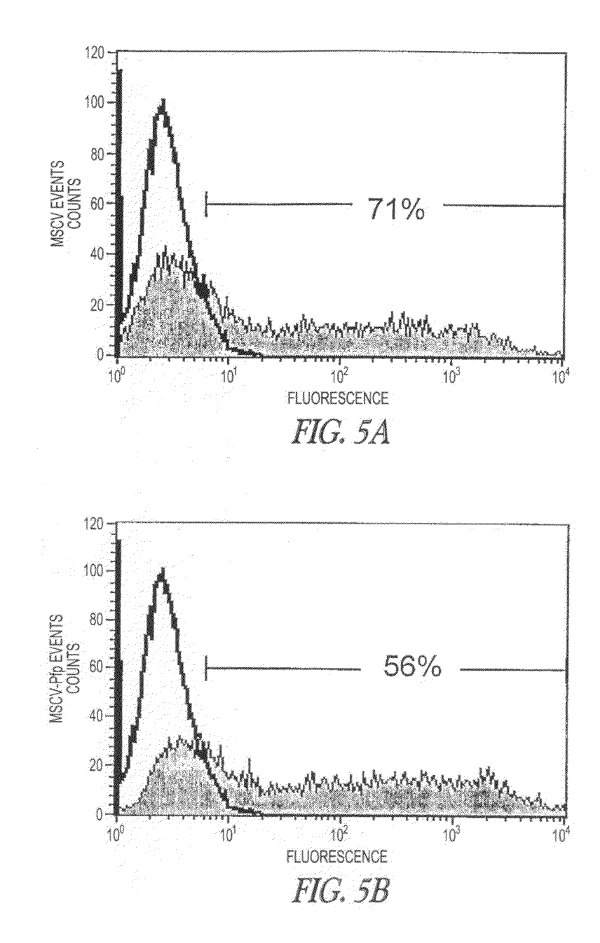

Expression of perforin was achieved in RBL cells using a retroviral-mediated approach based on the MSCV vector. Transfection of 293T packaging cells with MSCV-Pfp constructs gave rise to culture medium enriched for retrovirus which was used to transduce RBL cells. Flow cytometry analysis of the 293T cells for GFP expression was assessed 3 days after transfection as an estimate of the efficiency of the transfection. It has previously been shown in extensive experiments that GFP expression in more than 30% of the 293T cells was a reliable indication that viral titres up to 1.5×10⁷ pfu/ml of infectious virus was present in the culture supernatant (Dr. S Jane, Royal Melbourne Hospital, personal communication). Formal viral plaque assays were therefore not rountinely performed. As shown in FIG. 5, more than 50% of the 293T cells transfected with the MSCV-Pfp plasmid were strongly fluorescent, indicating GFP expression, 3 days after transfection. This was comparable to the GFP expression levels seen with empty-MSCV vector DNA. As expected, 293T cells transfected with the helper plasmid alone (blue solid line) did not express GFP.

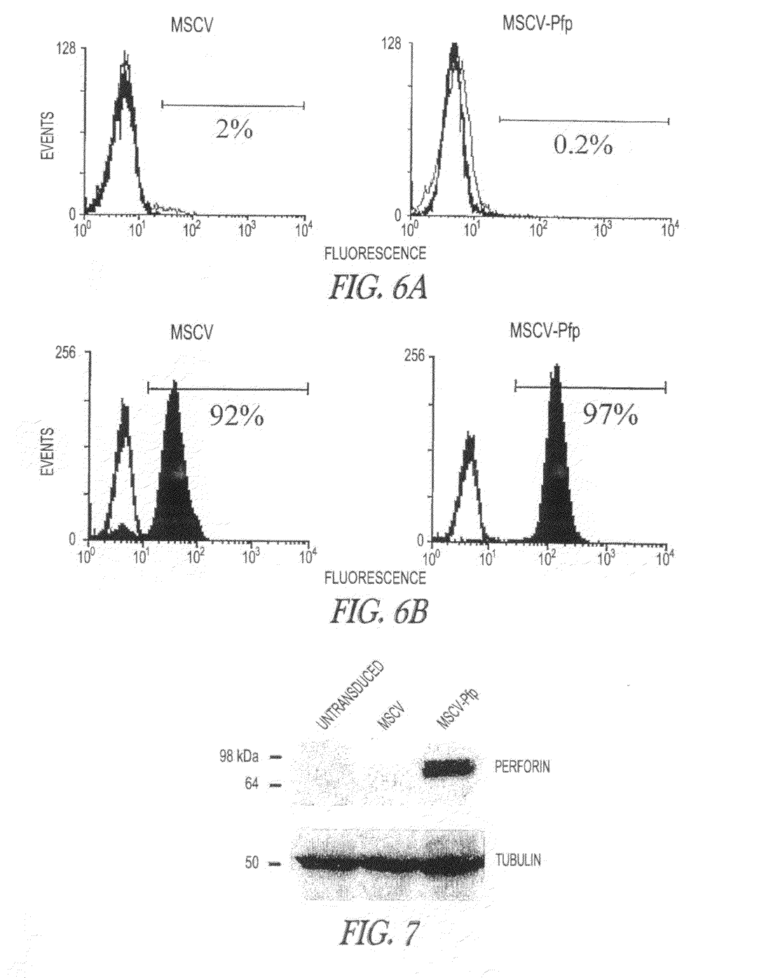

Next, RBL cells were transduced with the viral supernatant derived from the 293T cell transfection. Flow cytometry analysis was then performed in a similar manner, utilising GFP as a marker for RBL cells expressing perforin. The histogram profiles in FIG. 6A revealed a small population (typically between 0.1 and 5%) expressing high levels of GFP, depicted as the M1 gated region. Both the empty MSCV- and MSCV-Pfp-infected cells were isolated and expanded, resulting in a population in which more than 95% of cells expressed the surrogate marker (FIG. 6B). Sequential analysis showed GFP expression to be stable in cells that were continually cultured for more up to 8 weeks.

RBL cells transduced to express MSCV-Pfp were then analysed by western blotting for protein expression. As shown in FIG. 7, a 67 kDa immunoreactive band corresponding to perforin was identified in the RBL cells transduced with MSCV-Pfp, but neither parental RBL, nor unmodified empty vector-transduced RBL cells showed any perforin expression.

B. Optimal Labelling Conditions of RBL Cells with IgE Antibody

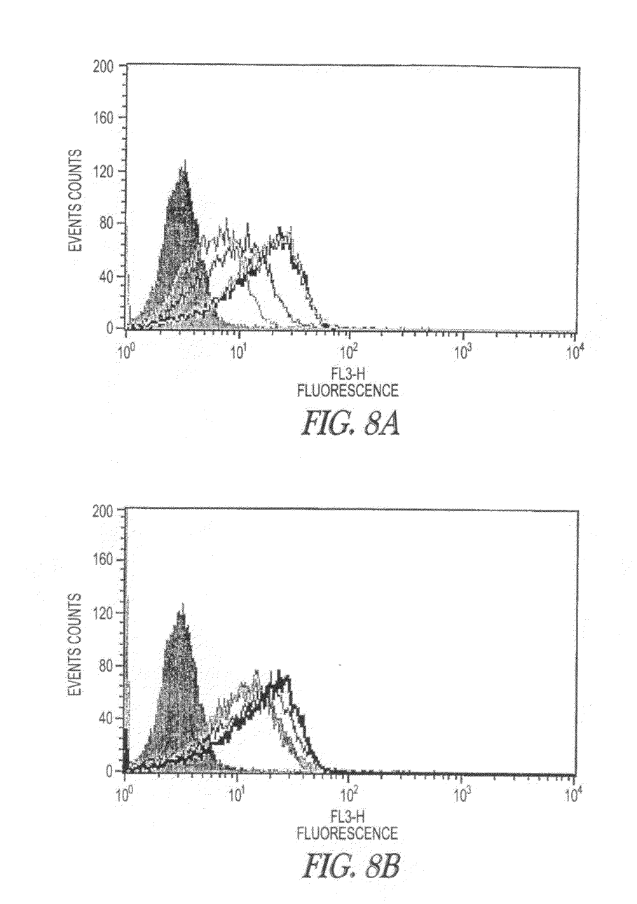

As a prelude to cytotoxicity assays, RBL cells were labelled with anti-TNP IgE antibody to determine the optimal conditions for IgE binding as a function of temperature, time and concentration of antibody. Flow cytometry analysis of RBL cells incubated with various concentrations of the antibody showed that a ½ or 1/10 dilution achieved a saturating level of binding (FIG. 8A). To label RBL cells for the purpose of the cytotoxicity assay (see next section), the ½ dilution was selected. FIG. 8B shows the level of antibody binding when temperature and incubation time were varied. The highest level of binding took place at 37° C. degrees for 1 hour, with almost equivalent binding at 4° C. degrees for 1 hour. Incubation for 15 minutes resulted in somewhat lower binding. To prepare RBL cells for the cytotoxicity assay, it was concluded that cells would be incubated in a ½ dilution of antibody at 37° C. for 1 hour.

C. RBL Cells Expressing MSCV-Pfp Acquire Strong Cytotoxicity

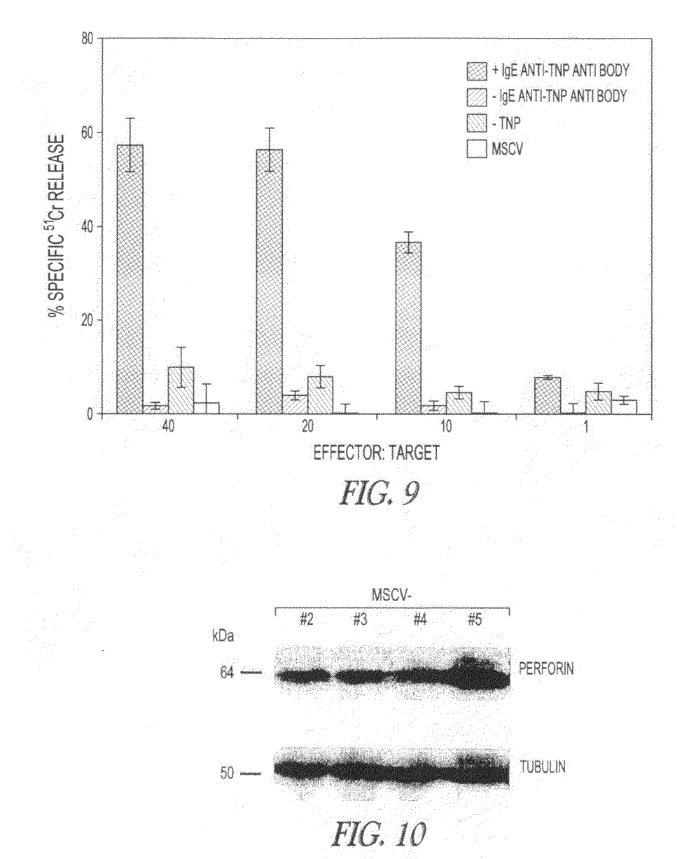

To test the cytolytic potential of RBL cells expressing MSCV-Pfp, these cells were labelled with anti-TNP IgE antibody and used as effectors cells to kill TNP-labelled target cells. Target cell death was assessed in a 4 hr ⁵¹Cr release assay. As shown in FIG. 9, RBL cells expressing perforin exhibited potent cytotoxicity against TNP-labelled EL-4 cells. At the highest E:T ratio of 40:1, RBL cells were able to induce about 60% specific ⁵¹Cr release and this level of cell death became reduced as the effector:target ratio fell. As expected, RBL cells transduced with an empty MSCV vector were not capable of lysing target cells, indicating that this non-cytotoxic cell line can be endowed with potent cytotoxicity when it expresses perforin. No lytic activity was observed with RBL cells in the absence of anti-TNP IgE antibody, indicating that efficient binding to the target cell and degranulation were essential for lysis. Similarly, EL-4 target cells that were not labelled with TNP failed to be recognised or killed by the effector cells. The cytotoxicity experiment was also repeated as a time-course assay to determine when maximal lysis of the target cells took place. Maximal ⁵¹Cr release was observed at 6 hours, with a plateau in ⁵¹Cr release observed beyond this time point, out to 24 hours. It was decided that the standard cytotoxicity assay would be carried out for 4 hours, as this timepoint resulted in a similar level of lysis observed after 6 hours.

D. Reproducibility of Perforin Expression: Production of Independent RBL Cells Lines Expressing MSCV-Pfp

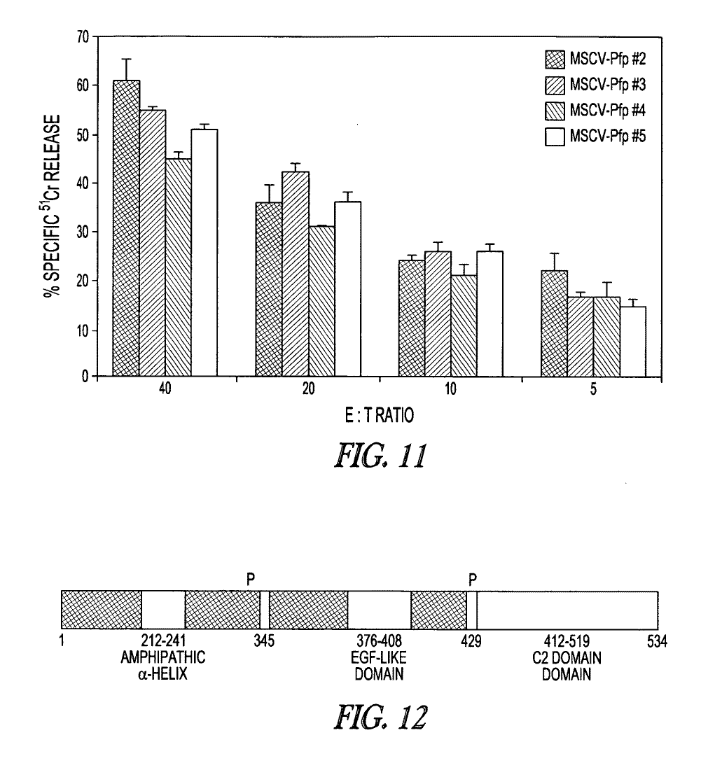

In order to assess the reproducibility of this method, multiple independent RBL cell lines expressing MSCV-Pfp were produced. The protocol described throughout this chapter was repeated: four further independent viral supernatants were generated by transfecting the MSCV-Pfp construct into 293 T cells and subsequently, RBL cells were transduced with the viral supernatants giving rise to RBL populations termed MSCV-Pfp #2-5. Western blotting revealed that all four RBL populations expressed approximately equal levels of perforin protein, however MSCV-Pfp #5, expressed slightly higher levels as compared to the tubulin loading control (FIG. 10). These populations were then used in a standard ⁵¹Cr assay to determine a normal range of lysis achieved by MSCV-Pfp (FIG. 11). Lysis was found to range between 40% and 60% for the four perforin-expressing populations at an effector:target ratio of 40:1. As expected, no significant lysis was observed by using negative control RBL cells discussed earlier in FIG. 8. To assess variations in cytotoxicity observed by the RBL cells on different days the assay was repeated multiple times (n=6) with all four populations, and a mean value of ⁵¹Cr release of 56%+/−3% was calculated at an effector:target ration of 40:1. In this way, mutant function to be investigated in future chapters can be compared to this standardised level of killing.

### Example 2

### Functional Analysis of Two Missense Perforin Mutations (G429E and P345L) by Retroviral Expression in RBL Cells

A. Construction of Mutated Mouse Perforin cDNAs

The mutations identified in Patient 5 (G429E) and in Patient 6 (P345L) (FIG. 12) were introduced into recombinant perforin cDNA for expression in RBL cells. MSCV plasmids encoding the mutated perforin cDNA will be referred to as P5-Pfp and P6-Pfp respectively. Using the wild type perforin cDNA inserted in MSCV (WT-Pfp) as a template (see Example 1), mutations were introduced using a site-directed mutagensis PCR reaction and the following primers:

For the introduction of the P5 (G429E) mutation:

| | |
|---|---|
| Sense: | |
| 5′AGAACATCTGTGGGAAGACTACACCACAG3′ | (SEQ ID NO: 3) |
| Antisense: | |
| 5′CTGTGGTGTAGTCTTCCCACAGATG3′ | (SEQ ID NO: 4) |

For the introduction of the P6 (P345L) mutation:

| | |
|---|---|
| Sense: | |
| 5′ CTACAGCCTGGAGCTCCTGCACACATTAC 3′ | (SEQ ID NO: 5) |
| Antisense: | |
| 5′ GTAATGTGTGCAGGAGCTCCAGGCTGTAG 3′ | (SEQ ID NO: 6) |

The PCR were set up according to manufacturer's instructions in the Quickchange Site Directed mutagenesis kit instructions (Stratagene, Calif.) and contained: 50 ng template DNA (WT-Pfp MSCV plasmid), 2.5 units Pfu Polymerase, 50 uM dNTP mix, 125 ng of each primer in Pfu Polymerase buffer. The PCR consisted of the following cycles: 1 cycle at 95° C. for 30 seconds, 14 cycles consisting of 95° C. for 30 seconds, 55° C. for 1 minute and 68° C. for 5 minutes (2 minutes/Kb plasmid length). Following completion, the PCR mixture was digested with 10 U of DpnI enzyme at 37° C. for 1 hour to digest parental DNA template while leaving newly synthesized mutated DNA intact. The DpnI endonuclease, which targets methylated and hemimethylated DNA was used to selectively digest the parental DNA. The PCR-derived DNA, incorporating the desired mutation was then used to transform XL-10 Gold supercompetent cells. For the transformation, 1 μl of digested DNA was added to 100 μl of competent XL-10 Gold competent cells and placed on ice for 30 minutes. Cells were heat-shocked at 42° C. for 45 seconds, placed on ice for 2 minutes and incubated with 200 μl LB-Broth, at 37° C. for 30 minutes before plating out on Amp LB agar plates.

Miniprep and large-scale DNA preparations were carried out according to the methods outlined in Example 1. cDNA clones were sequenced to verify that only the desired mutations had been introduced (see Example 1 for sequencing protocols). P5-Pfp and P6-Pfp inserts were then subcloned into EcoRI-XhoI digested MSCV vector DNA.

B. Expression of Mutated Perforin Protein in RBL Cells.

The expression of P5-Pfp and P6-Pfp in RBL cells was achieved using the protocol optimised in Example 1. Briefly, this involved transfection of 293T cells for the generation of virus-enriched supernatant, transduction of RBL cells and cell sorting for isolation of RBL cells expressing high levels of the GFP marker. Whole cell lysates were analysed for protein expression as hereinbefore described in Example 1.

C. Comparison of the Function of P5 and P6 Mutated Pfp to WT-Pfp Cyolytic Function

The cytolytic function of RBL cells expressing P5-Pfp or P6-Pfp was analysed against EL-4 target cells in a 4 hour ⁵¹Cr release cytotoxicity assay as previously outlined (Example 1). WT Pfp-expressing RBL cells described in Example 1 were used as the positive control for perforin function, and RBL cells transduced with empty MSCV vector as a negative control.

D. Isolation of Lysosomal Granules from RBL Cells

WT-Pfp, P5-Pfp or P6-Pfp was isolated from the RBL granules by nitrogen caviatation and percoll density fractionation of the cellular contents as described by Davis et al (J Immunol Methods. 2003, 276(1-2):59-68). RBL cells (1×10⁹) were washed three times in PBS, then resuspended at 1×10⁸/ml in relaxation buffer (100 mM KCL, 3.5 mM MgCl2, 1 mM PIPES pH6.8, 1.25 mM EGTA) and lysed in a nitrogen cavitation apparatus at 450 psi for 20 minutes at 4° C. on a rotating platform. The cell lysate was collected following sudden decompression, and the nuclei removed by centrifugation at 2000 rpm for 10 minutes at 4° C. The nuclei were washed twice with 1 ml relaxation buffer and the supernatants were pooled with the supernatant from the first wash. The pooled supernatants were centrifuged at 2000 rpm for a further 5 minutes to remove all cell debris. A 40% percoll density gradient was then formed by mixing 8 ml of adjusted percoll (45 ml percoll and 5 ml 10× relaxation buffer) with 12 ml of relaxation buffer, containing 1 mM ATP. 5 ml of cell lysate was loaded onto each gradient and centrifuged at 20,000 rpm for 35 minutes at 4° C. The cytotoxic granules, which migrate to the dense region of the gradient, were collected by harvesting 1 ml fractions from the bottom of the gradient using a long spinal tap needle attached to a syringe. The fractions were concentrated (individually) in an ultracentrifuge (Beckman Coulter) at 100,000 rpm for 3 hours at 4° C. and the granules obtained from the surface of the pelleted percoll by washing them into a small volume of resuspension buffer. To release the perforin, the granules were disrupted by resuspension in an equal volume of 2 M NaCl and three cycles of freezing in liquid nitrogen and thawing in a 37° C. waterbath.

(i) β-Hexoaminidase Assay

50 ul of freeze-thawed granule extract was added to 30 ul of 8 mM p-nitrophenyl N-acetyl-β-D.glucosaminide (Sigma) in H₂O and 10 ul of 0.5 M sodium acetate solution (pH 5.0). The reaction was stopped after 30 minutes at room temperature by the addition of 150 ul of 50 mM NaOH and the optical density of the samples measured at 405 nm.

(ii) Lysis of Jurkat Cells by Granule Extracts

Jurkat cells were labelled with 50 μCi ⁵¹Chromium in 100 ul unsupplemented RPMI medium at 37° C. for 1 hour. ⁵¹Cr-labelled cells were then resuspended in HBSS buffer (CSL Ltd.) with or without 2 mM EGTA. For the assay, 2×10⁴ cells resuspended in Hank's buffered saline solution (HBSS) were added in wells of a 96 well V-bottom plate. Granule fraction #8 (determined by western analysis to contain the highest perforin content) was serially diluted in HE buffer and incubated with target cells in a final volume of 200 for 4 hours at 37° C. The spontaneous release of ⁵¹Cr was determined by incubating the target cells with HE buffer alone and the maximum release was determined by adding HCl to a final concentration of 1M. After 4 hours, the plates were centrifuged at 1500 rpm, 100 μl of supernatant was harvested and the released radioactivity was measured in a Wizard 1470 Gamma counter. Cytotoxicity was expressed as a percentage specific ⁵¹Cr release after subtracting spontaneous release. The percent specific lysis was calculated as follows: 100×[(experimental release−spontaneous release)/(maximum release−spontaneous release)].

(iii) Erythrocyte Lysis Assay

Sheep red blood cells (sRBC) were washed three times then resuspended at 10⁸/mL in 150 mM NaCl. For the assay, 50 ul of freeze/thawed granule extract [fraction #8 (see above)] was incubated with 20 μl of the sRBC suspension in the presence of 2 mM CaCl₂ at 37° C. for 30 minutes in v-bottom 96 well plates. For maximal haemoglobin release, H₂O was used to lyse the red blood cells. Plates were centrifuged at 1500 rpm for 5 minutes and the haemoglobin released into the supernatant was estimated by measuring the optical density at 405 nm. Cell lysis was expressed as a percentage of maximal haemoglobin release.

(iv) Immunoperoxidase Staining for Perforin in RBL Cells

Approximately 1×10⁵ cells were seeded in each well of an 8 well chamber slide one day prior to the staining procedure. Cells were fixed for 10 minutes at room temperature in fixation buffer (3.7% paraformaledehyde in PMED) and then washed three times in PBS. Permeabilisation buffer (0.1% Triton-X, 0.5% BSA) was then added for 5 minutes and the cells were washed as before. The cells were treated with Periodic acid (0.5%) for 10 minutes at room temperature, rinsed and endogenous peroxide quenched by incubating with 0.3% H₂O₂ for 15 minutes. Blocking buffer (1% BSA/1% skim milk powder/PBS) was added to the wells for 30 minutes and washed twice as before. The monoclonal anti-mouse perforin antibody, P1-8, was then added ( 1/1000 dilution or 2 μg/ml). After 3 washes in PBS, a biotin-conjugated donkey anti-rat IgG antibody (Jackson ImmunoResearch, USA) was added ( 1/600) dilution and the cells washed as before. Streptavidin-HRP (Dako) was incubated with the cells for 10 minutes at room temperature, washed three times and the HRP signal detected by adding the chromogen DAB (Dako) for a further 10 minutes. Cells were counterstained with eosin for visualization of the nucleus.

(v) Degranulation of RBL Cells

RBL cells were triggered to exocytose their granule contents to assess the release of perforin. Empty-MSCV transduced RBL cells, or cells expressing WT-Pfp, P5-Pfp or P6-Pfp (1×10⁵) and were seeded in wells as described above, and labelled with anti-TNP IgE antibody (½ dilution in PBS) for 30 minutes at 37° C. Cells were washed three times in PBS then 1×10⁶ TNP-labelled EL-4 cells (see Example 1) were added to the effector cells and incubated for 30 minutes at 37° C. Cells were then washed three times with PBS to remove the EL-4 cells. To compare their perforin content before and after degranulation, RBL cells were immunostained as described earlier.

A. Expression of Perforin in RBL Cells Using Retroviral Expression System

The aim of the current study was to use the RBL expression system to characterise the biosynthesis and function of two mutated forms of perforin expressed in FHL patients, P5 and P6. Therefore, using the methodology optimised in Example 1 for the expression of WT mouse perforin, mutations equivalent to the human P5 and P6 mutations were introduced into mouse perforin for expression in the RBL cells. The residues in question (G429 and P345) are invariant in human, mouse and rat perforins, suggesting conservation of function.

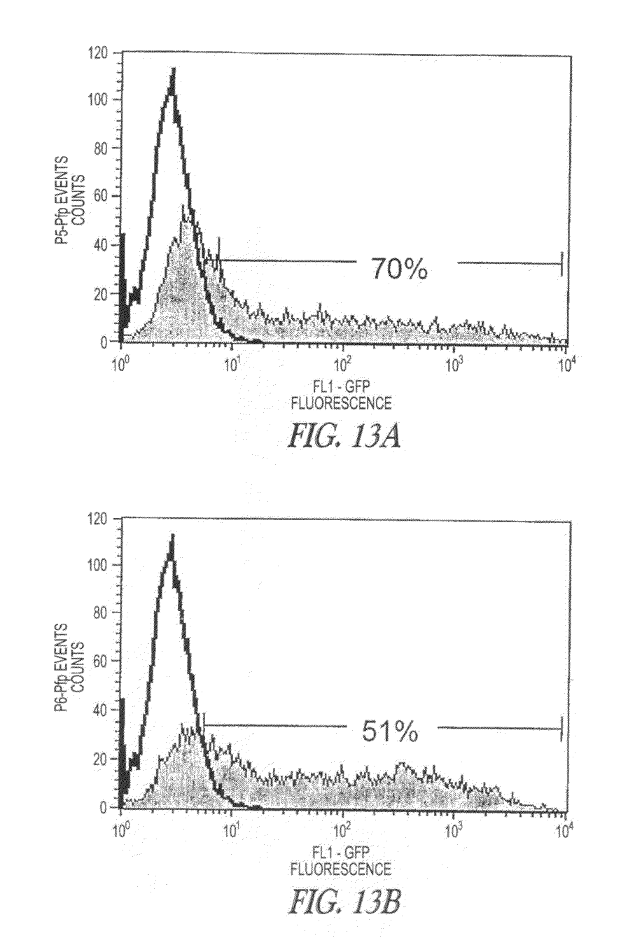

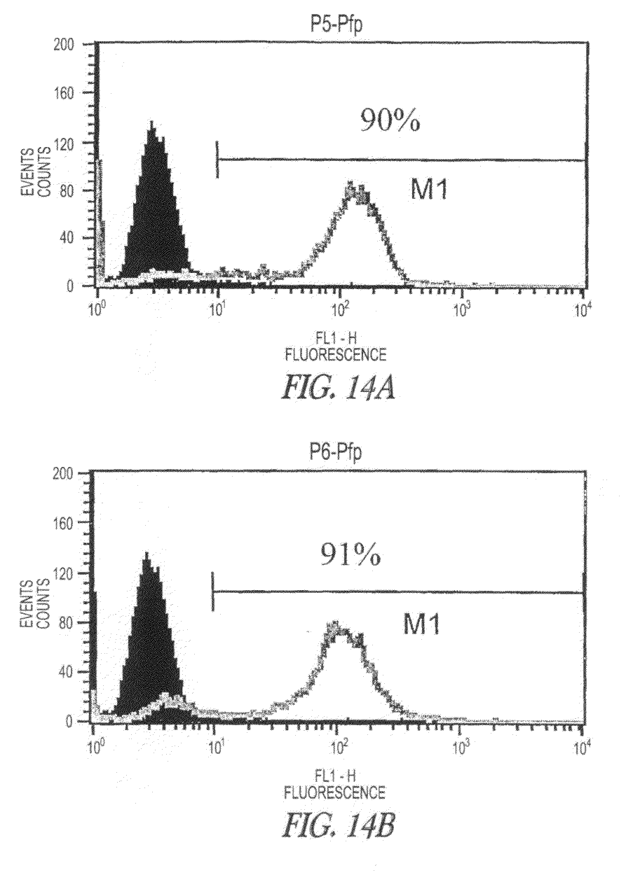

The two-step retroviral transduction procedure once again involved initial transfection of 293T cells with plasmid DNA, giving rise to enriched viral supernatant required for the transduction of RBL cells. Analysis of the 293T cells following transfection with the P5-Pfp and P6-Pfp expression constructs indicated that more than 50% of the cells were expressing GFP, suggesting a high virus titre in the culture supernatant (FIG. 13). The levels of fluorescence were comparable to those seen in 293T cells transfected with the WT-Pfp and empty-MSCV constructs (see Example 1 and FIG. 5). Following retroviral transduction, small populations of RBL cells expressing the GFP marker were once again sorted and re-expanded in culture to yield a population of cells with uniformly high expression of the GFP transgene. Analysis of the expanded populations confirmed the selection of GFP-expressing cells, in that more than 90% of cells transduced with virus encoding P5-Pfp and P6-Pfp were now strongly fluorescent (FIG. 14). Taken together, these expression profiles indicated that the expression of mutated perforin occurred in a manner similar to WT-Pfp (see Example 1 for WT-Pfp expression).

Western blot analysis of the expanded RBL populations with the P1-8 mAb detecting perforin, revealed the perforin protein with apparent molecular weight of 67 kDa in each cell population transduced to express P5-Pfp and P6-Pfp perforin but not in cells transduced with empty vector (FIG. 15). The mutated perforin protein was expressed to similar levels as the WT-Pfp, as compared with tubulin loading controls, suggesting that introducing the respective FHL mutations into perforin did not affect the stability of the protein in the RBL cells.

B. Cytotoxicity Mediated by RBL Cells Expressing Wt and Mutated Pfp

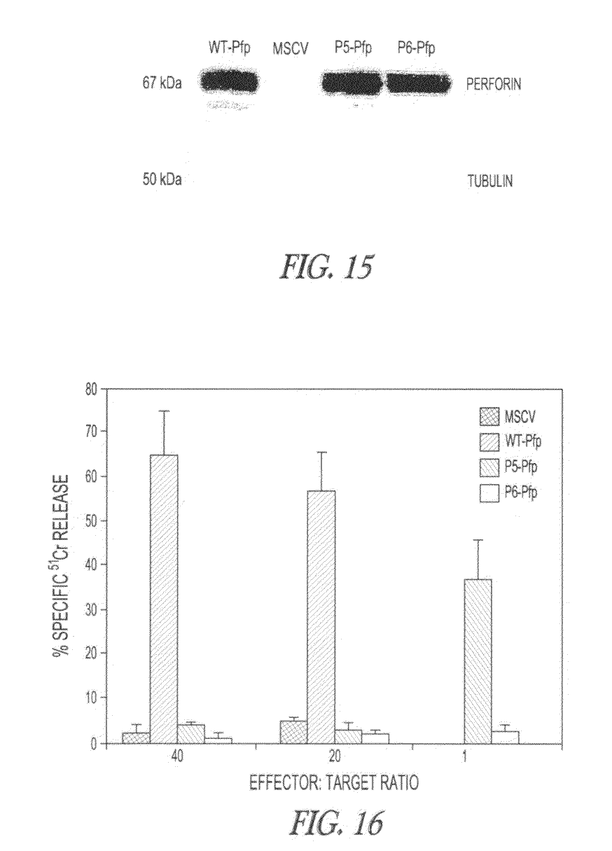

To test the effect of the introduced mutations on perforin function, the cytolytic capacity of the RBL cells expressing P5-Pfp or P6-Pfp was compared to cells expressing WT-Pfp in a 4 hr ⁵¹Cr release assay using TNP-labelled EL-4 cells as targets (FIG. 16). In marked contrast to the potent cytotoxicity seen with RBL cells expressing WT-Pfp, the release of ⁵¹Cr release from target cells in response to RBL populations expressing mutated perforin was greatly reduced. This result was reproduced in several experiments and at multiple E:T ratios.

The lack of cytotoxicity observed could not be attributed to differences in expression levels of the protein, as shown earlier by western blotting.

C. WT and Mutated Perforin are Localised in RBL Cytoplasmic Granules

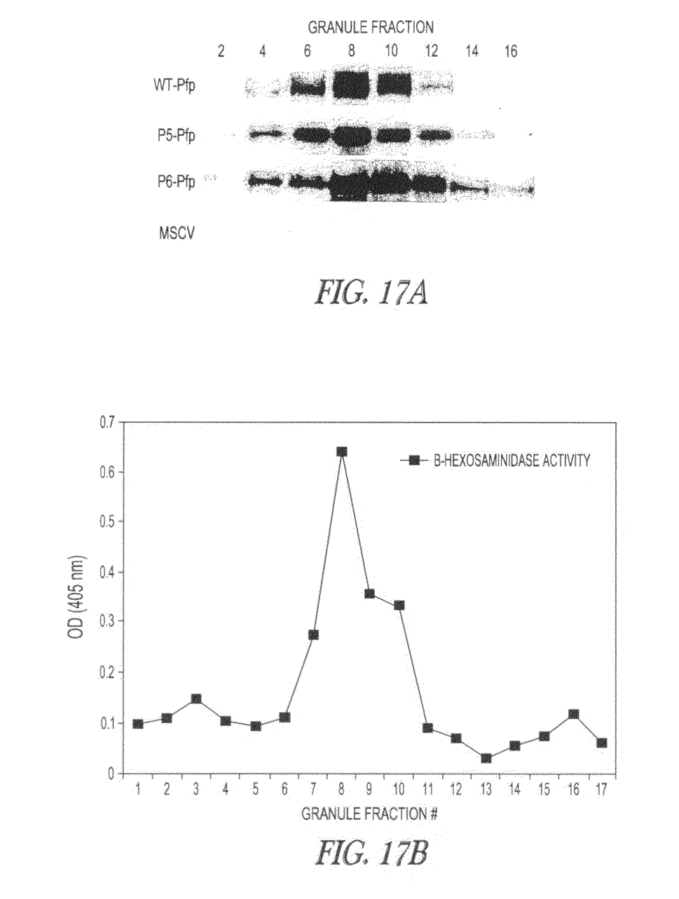

The subcellular distribution of perforin in RBL cells was examined in order to detect any differences in the trafficking of P5 or P6 mutant perforin to lysosomal granules, compared to WT-Pfp. Western blotting of the Percoll-fractionated RBL cell lysates showed perforin localisation in Fractions #6-10, with the peak perforin content in Fraction #8 (FIG. 17A). Peak perforin expression also coincided with maximum β-hexo-glucosaminidase activity (FIG. 17B), an enzymatic marker of the lysosomal granules (Schwartz and Austen, 1980; J Invest Dermatol. 1980, 74(5):349-53). This indicated that WT and mutated perforins were localised within the secretory granules and lysosomes. These findings were also concordant with previous data in which fractions 6-8 were identified as the granule-rich fractions of RBL cells.

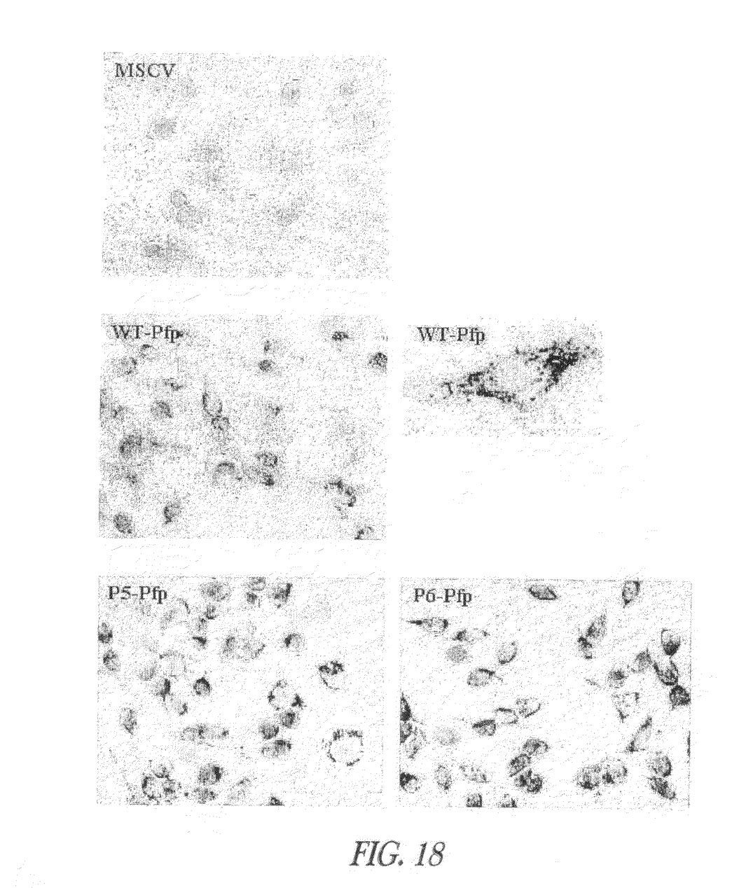

The subcellular localisation of perforin to secretory granules was further confirmed by immunhistochemical staining. As shown in FIG. 18, RBL cells expressing WT-Pfp, P5-Pfp and P6-Pfp stained strongly for perforin, whereas empty vector-transduced RBL cells did not show any staining. Virtually 100% of the cells stained for perforin which was consistent with earlier flow cytometry analysis for GFP expression, which was found in more than 95% of the RBL cells (FIG. 15). Under high magnification punctate cytoplasmic staining was observed, consistent with lysosomal localisation of the perforin. Similar punctate staining was also observed under high magnification for the mutated P5-Pfp and P6-pfp.

D. Investigating the Degranulation Function of P5-Pfp and P6-Pfp: Lysis of Nucleated and Enucleated Target Cells by Granules Contents

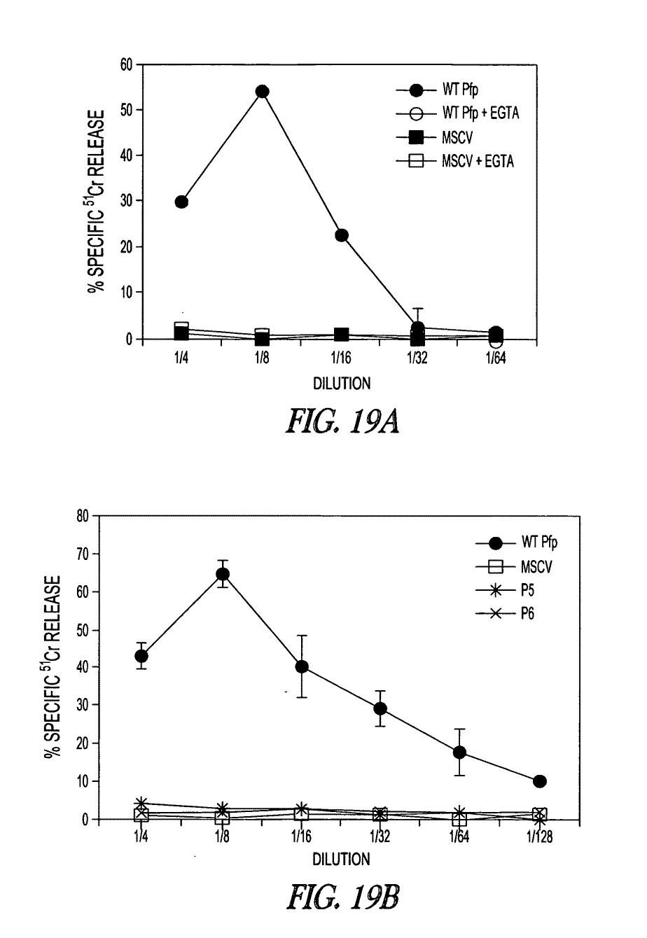

The results presented above suggested that P5-Pfp and P6-Pfp were both synthesised, trafficked and stored normally in cytoplasmic granules, and that each mutated form is incapable of inducing target cell death. However, it was also possible that both mutated perforins were incapable of being released from the RBL cells by exocytosis. This possibility was tested by purifying the lytic granules and applying them directly to target cells. Thus, P5-Pfp and P6-Pfp were dissociated from their intracellular compartment, bypassing a potential defect in degranulation. WT and mutated perforins were then tested for their ability to lyse nucleated Jurkat cells and non-nucleated sRBC. As shown in FIG. 19A, WT-Pfp caused considerable lysis of Jurkat cells, with a clear dose-dependent effect as the granules were diluted. At the highest concentration of granules tested, a somewhat lower level of cytotoxicity was observed, possibly due to the presence of some inhibitory granule component, perhaps acting as a scavenger of calcium ions. By diluting the granules 1/32, approximately 65% specific lysis was observed. This lytic function was completely inhibited by the addition of Ethyleneglycotetraacetic acid (EGTA), a chelator of calcium ions, indicating that lysis was proceeding through a perforin-mediated mechanism. Granules derived from empty-MSCV transduced RBL cells did not induce lysis of the Jurkat cells. In striking contrast to WT perforin, the P5-Pfp and P6-Pfp containing granules were incapable of causing any damage of the target cells in the presence of Ca²⁺ (FIG. 19B).

In a similar experiment, the disrupted granules were mixed with sRBC which are non-nucleated and are more sensitive to perforin-mediated membrane damage (Shiver and Henkart, Cell. 1991, 64(6):1175-81). WT-Pfp resulted in almost complete RBC lysis as detected by haemoglobin release at a ⅛ dilution and significant lysis was seen out to 1/64 (FIG. 19C). Hemolysis was a function of the amount of granule material added in the assay and was inhibited by EGTA, as with Jurkat cell targets. Neither P5-Pfp, nor P6-Pfp containing granules were able to cause lysis of the sRBC.

E. Investigating the Degranulation Function of P5-Pfp and P6-Pfp: Visualisation of Perforin Content in Cytoplasmic Granules Before and after Degranulation

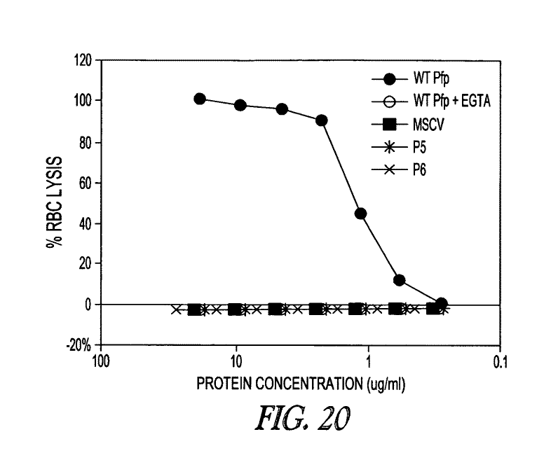

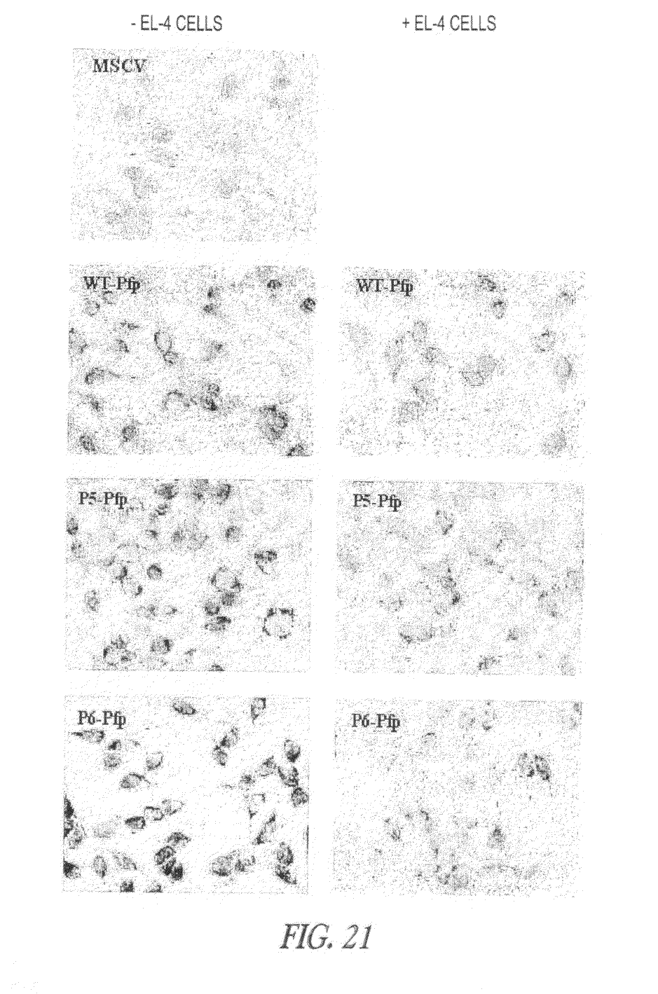

The ability of WT-Pfp, P5-Pfp and P6-Pfp to be liberated from RBL cells was examined directly using immunohistochemistry. RBL cells were stimulated to release their granules by labelling them with the anti-TNP IgE antibody and incubating them with TNP-labelled EL-4 cells. Unstimulated RBL cells expressed approximately equal quantities of WT-Pfp, P5-Pfp and P6-Pfp (FIG. 20). Following incubation with TNP-labelled EL-4 cells, the level of staining decreased significantly in the RBL cells, indicative of perforin exocytosis. This decrease in staining was similar whether WT-Pfp, P5-Pfp or P6-Pfp were expressed. This suggested that P5 and P6 perforin were equally capable of being exocytosed from the granules, and that the lack of cytotoxicity observed was due to perforin dysfunction at the level of the target cells.

### Example 3

### Functional Analysis of Two Putative Polymorphisms (R225W and G429E) Associated with Familial Hemophagocytic Lymphohistiocytosis

This study elucidates the cellular basis for perforin dysfunctions in hemophagocytic lymphohistiocytosis and demonstrates the utility of aspects of the present invention as a means for studying the “structure-function” relationship of perforin.

A. Cell Culture.

The cell lines RBL-2H3 cells (rat basophil leukemia; American Type Culture Collection), which will be referred to in the text as RBL, and 293T (human embryonic kidney) were maintained in DMEM medium supplemented with 10% FCS, 2 mM glutamine, and 100 μg/ml each of streptomycin and penicillin in a humidified incubator at 37° C. Jurkat T cells were maintained in RPMI-1640 medium supplemented as above. RBL and 293T cells were detached from culture flasks using trypsin-EDTA solution (CSL Ltd.) at 37° C.

B. Transient Transfection of RBL Cells.

Mature human and mouse perforin each have 534 amino acids. However, the leader sequence of human perforin is one amino acid longer than that of the mouse. This results in a difference in conventional amino acid numbering such that amino acids at positions 225 and 429 mutated in HLH Patient #5 (as described by Stepp, S. E. et al., 1999, Science, 286:1957-1959) correspond to residues 224 and 428 in the mouse protein, as noted in the experiments below. Importantly, arginine 225 is a nonconserved residue with threonine being present in mouse perforin. To demonstrate the equivalence of arginine and threonine at this position, we generated the T224R variant and, subsequently, the T224W mutant, which corresponds to R225W in Patient #5 (11). The mutations were introduced using the Transformer (Stratagene) site-directed mutagenesis system according to the manufacturer's instructions. The resultant and the WT cDNA was cloned into the pIRES2-EGFP expression vector (CLONTECH Laboratories, Inc.). Fcε receptor-expressing RBL cells were grown to near confluence in 175-cm² flasks, harvested, washed twice, and resuspended at 10⁷ cells/ml in serum-free DMEM. 200 μL of the cell suspension was mixed with 20 μg pIRES2-EGFP containing the WT or mutated perforin cDNA or vector DNA alone, incubated at room temperature for 10 min, and electroporated in 4-mm electroporation cuvettes and Bio-Rad Laboratories pulser at 500 μF and 0.25 V. After 10 min at RT, the cells were transferred into complete DMEM. Cells were harvested 18-20 h later, and GFP-expressing cells were sorted by flow cytometry (FACStar; Beckton Dickinson).

C. Generation of Recombinant Retroviruses and Stable Expression of Perforin in RBL Cells.

The missense perforin mutation, G428E, corresponding to the human G429E (identified in another perforin allele in Patient #5), was generated using the Quick-Change site-directed mutagenesis system (Stratagene) according to the manufacturer's instructions. The cDNAs encoding mouse WT and G428E perforin were subcloned into the retroviral expression vector MSCV, which contains an internal ribosome entry site for GFP expression. For retroviral transduction of RBL cells, viral supernatant was generated by cotransfecting the MSCV plasmids with an amphotropic packaging plasmid into 293T cells by calcium phosphate precipitation.

After 48 h, the viral supernatant was harvested and added to RBL cells every 12 hours for 3 days. The population of cells with the greatest GFP expression (up to 5% of total cells) was subsequently purified by flow cytometry and analyzed for perforin expression.

D. Assessing the Cytotoxicity of Transfected RBL Cells.

The cytotoxic capacity of RBL cells was analyzed using Jurkat T cell targets in a 4-h ⁵¹Cr release assay, as detailed above. Briefly, the surface of ⁵¹Cr-labeled Jurkat cells was derivatized with a 1 mM solution of trinitrobenzosulfonic acid in PBS, (pH 7.4) for 15 min at 37° C. and washed with unsupplemented DMEM three times. The transfected RBL cells were harvested and incubated with antitrinitrophenol IgE mAb (2 μg/ml) at 37° C. for 15 min and washed with unsupplemented DMEM three times. RBL and Jurkat cells were coincubated at various effector to target (E:T) ratios at 37° C. in 2004 serum-free DMEM supplemented with 1% BSA for 4 h in 96-well plates. The supernatant was then harvested and the released ⁵¹Cr measured in a gamma counter. The total ⁵¹Cr content of Jurkat cells was estimated using 5% Triton X-100-lysed cells. The percentage-specific chromium release was calculated as 100×([experimental release×spontaneous release]/[total release−spontaneous release])) and is shown as mean±SD.

E. Isolation of Lysosomal Granules from RBL Cells.

Perforin was isolated from 10⁹ stably expressing RBL cells by nitrogen cavitation and Percoll density fractionation. To distinguish granule-enriched fractions from other subcellular fractions, the activity of the RBL granule marker enzyme, β-hexosaminidase, was measured as follows. 50 μL of each fraction was mixed with 30 μL 8 mM p-nitrophenyl N-acetyl-β-D-glucosaminide (Sigma-Aldrich) and 10 μL 0.5 M sodium acetate, pH 5.0, at RT for 30 min. The reaction was stopped by adding 150 μL 50 mM NaOH, and the absorbance was measured at 405 nm.

F. Expression of Recombinant Perforin and Membrane-Binding Assay.

Perforin cDNA was cloned into the pFastBac vector and overexpressed in Sf-21 cells cultured in SF900-II SFM medium using a Bac-to-Bac kit (Invitrogen) and perforin was purified, all according to the manufacturer's instructions. Small amounts of recombinant WT and the G428E perforin mutant protein were obtained.

To study the calcium-dependent membrane binding of perforin, 2×10⁶ sheep RBCs were resuspended in 200 μL 20 mM Hepes-150 mM NaCl buffer (pH 7.4) supplemented with 1 mM CaCl₂. An aliquot of the purified perforin was added to the cell suspension for 5 min on ice. The cells were pelleted at 16,000 g for 10 s, the supernatant promptly removed, and the cells lysed in ice cold water. The lysate was centrifuged for 20 min at 16,000 g at 4° C. The pellet was washed once, dissolved in SDS-PAGE loading buffer, and analyzed by Western blotting.

G. Immunoperoxidase Staining.

Approximately 1,000 RBL cells were seeded in each well of an 8-well chamber slide 1 day before staining and cultured overnight. In some experiments, cells were induced to undergo degranulation by transient incubation with TNP-labeled tumor target cells. The RBL cells were fixed for 10 min at RT in 3.7% paraformaldehyde, washed three times in PBS, permeabilized in 0.1% Triton X-100, 0.5% BSA for 5 min, and then washed as before. The cells were treated with periodic acid (0.5%) for 10 min, and endogenous peroxidase activity was quenched with 0.3% H₂O₂ for 15 min. Blocking buffer (1% BSA/1% skim milk powder in PBS) was added for 30 min before the rat antiperforin mAb P1-8. Bound Ig was detected with biotinylated donkey anti-rat IgG (Jackson ImmunoResearch Laboratories), streptavidin-HRP (Dako) for 10 min, and the chromogen diaminobenzidine (Dako). Finally, cells were counterstained with eosin and viewed by light microscopy.

H. Western Blotting.

Cell lysates from stable or transiently transfected RBL cells or granule extracts were resolved on 10% SDS-PAGE (Tris-Glycine) gels, transferred to PVDF membranes, and assayed for perforin content using rat antiperforin mAb PI-8 and anti-rat HRP-conjugated Ig. The signal was detected using chemiluminescence (Amersham Biosciences).

G. Results

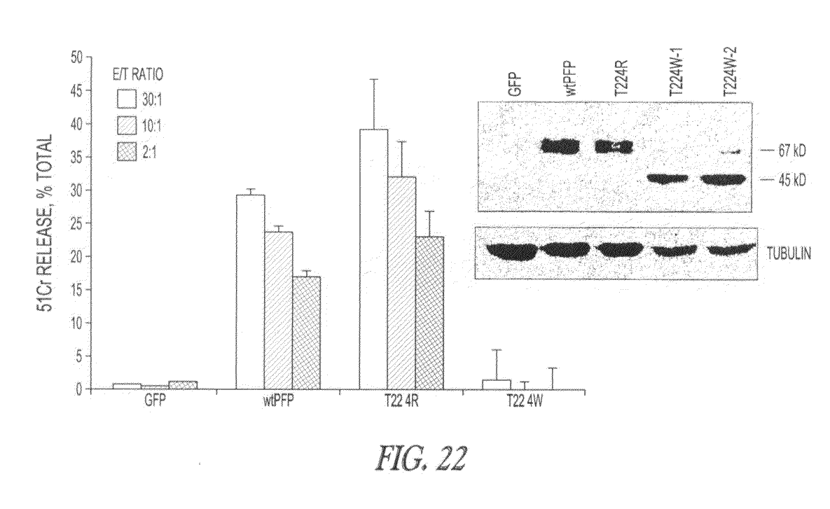

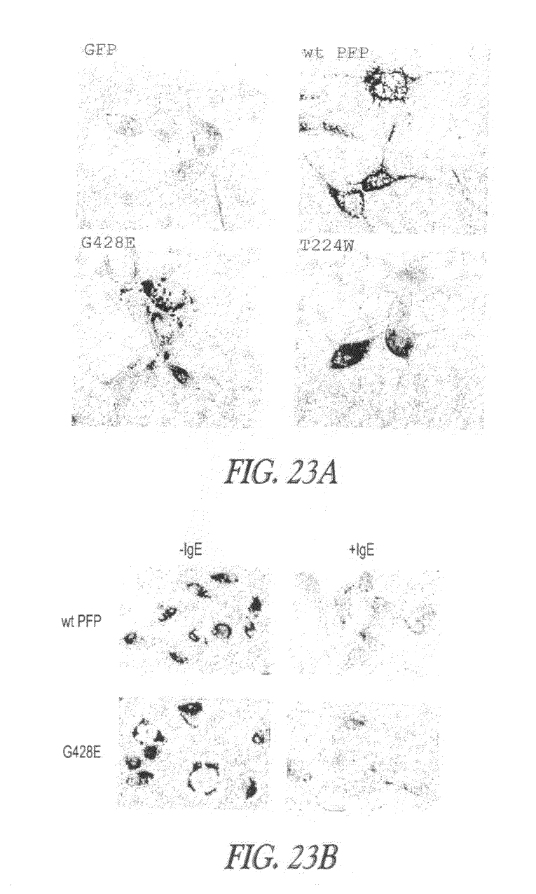

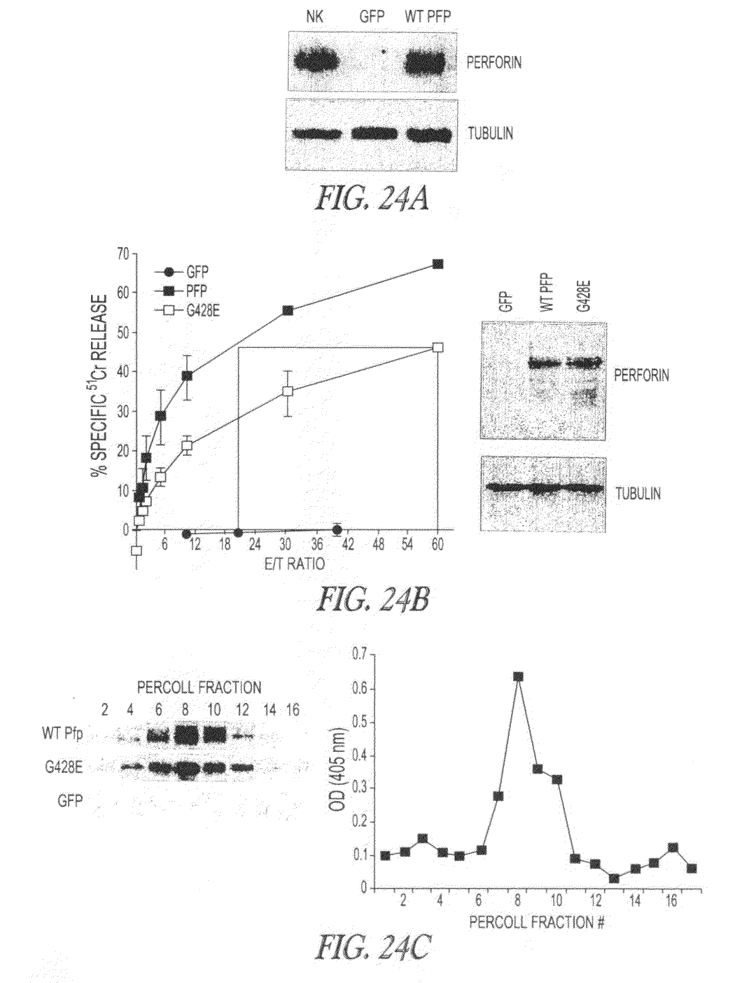

The efficiency of electroporation was as high as 40%, and up to 10⁶ GFP-expressing cells were obtained per electroporation. Although G429 is conserved in human, mouse, and rat perforin, R225 is not invariant and corresponds to T224 in mouse perforin. To confirm the functional equivalence of arginine and threonine at this position, we generated RBL cells expressing T224R mouse perforin and found they were as efficient in the ⁵¹Cr release assay as WT perforin-transfected cells. However, expressing perforin with tryptophan at the same position (T224W) resulted in complete loss of cytolytic function (FIG. 22). As expected, the WT protein had an apparent molecular mass of ˜67 kD; however, the introduction of tryptophan resulted in the appearance of truncated (˜45 kD) perforin (FIG. 22), suggesting the mutation facilitated proteolytic cleavage/processing of perforin. Furthermore, immunohistochemistry analysis of transfected cells indicated mislocalisation of T224W, possibly due to a loss of putative signaling motif(s). Whereas WT perforin produced a punctate appearance consistent with packaging in secretory granules, T224W perforin produced diffuse staining throughout the RBL cell cytoplasm (FIG. 23 A). When we similarly analyzed the effect of the G428E (G429E in humans) mutation co-inherited by Patient #5, we observed a reduced level of ⁵¹Cr release compared with RBL cells expressing WT perforin (data not shown). To accurately quantify this reduced activity, we produced cell lines that stably expressed WT and G428E perforin. Retrovirus-transduced RBL cells were analyzed on a flow cytometer, and the most highly fluorescent cells (0.2-5% of the total population) were sorted and expanded in culture resulting in ˜93% GFP-positive cells some days later. These cells expressed perforin at levels equivalent to IL-18/IL-21-activated mouse primary NK cells (FIG. 24 A). Perforin expression and cytotoxic function remained stable over many weeks of continuous culture (not depicted). Consistent with our transient transfection experiments, RBL cells expressing WT perforin were efficient in lysing Jurkat target cells across a broad range of E:T ratios (FIG. 24 B). To determine the difference in cytolytic activity between WT and G428E perforin, the E:T ratios required to produce equivalent levels of ⁵¹Cr release were compared. We found that RBL cells expressing similar levels of G428E were three to four times less efficient at inducing chromium release (FIG. 24 B).

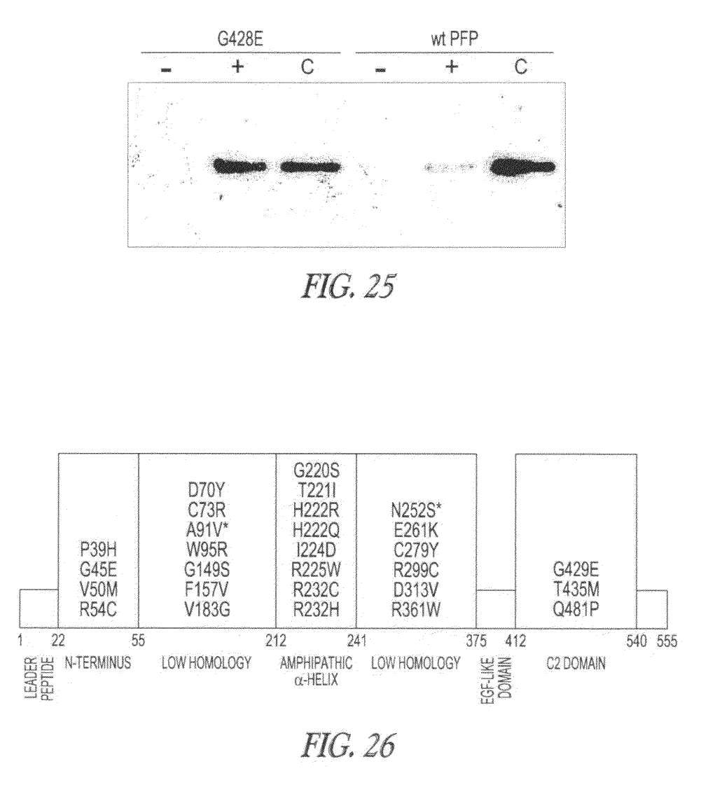

We then went on to investigate the reason for the reduced cytotoxicity of G428E perforin. As demonstrated by immunoblotting (FIG. 24 B), this was not due to protein cleavage or degradation. To rule out incorrect trafficking to secretory granules, we examined the intracellular localization of WT and G482E perforin in stably transduced RBL cells. Finding normal quantities of mutated perforin in the granules would further exclude a significant defect in gene transcription, mRNA stability or translation, or protein folding. When lysates of RBL cells expressing WT or G428E perforin were fractionated on a Percoll gradient and analyzed by Western blot, perforin was consistently localized in the fractions containing maximal β-hexosamidase activity, a marker of the lysosome-like secretory granules (FIG. 24 C). The correct targeting of perforin was also confirmed through immunohistochemical staining, as both WT and G428E perforin demonstrated indistinguishable punctate cytoplasmic staining (FIG. 23 A). G428E perforin was also released by exocytosis as efficiently as WT perforin upon RBL Fcε receptor cross-linking (FIG. 23 B). Since G428E perforin was expressed at equivalent levels to WT perforin and correctly targeted to, and released from, granules (FIG. 23 and FIGS. 24, B and C), the mutation was likely to affect a postsynaptic function of perforin. To test this possibility, we generated and purified recombinant WT and G428E perforin using a baculovirus expression system and tested their ability to bind to sheep RBC membranes in a calcium-dependent manner. Whereas WT perforin displayed strong calcium-dependent plasma membrane binding with essentially all the added perforin bound, the binding of G428E perforin was markedly reduced (FIG. 25). Consistent with this observation, the cytolytic activity of the recombinant G428E mutant was ˜5% of that of WT perforin (not depicted). Although RBL cells have been used as a read-out of perforin function for many years, a perceived weakness of the model is that perforin exerts its cytolytic effects in the absence of granzyme B. Exposure of target cells to recombinant G428E-perforin with granzyme B did not rescue the perforin phenotype (not depicted). Therefore, our findings strongly suggested that the diminished activity of G428E perforin was due to diminished target cell membrane binding, rather than the absence of granzymes.

This is the first study to successfully define the functional basis of naturally occurring perforin mutations that when co-inherited, lead to the catastrophic immunosuppression seen in HLH. Surprisingly, we demonstrated that partial loss of perforin function may be sufficient to bring about fatal disease. Whereas the T224W mutation (corresponding to R225W in humans) resulted in protein instability and complete loss of RBL cytotoxic function, G428E (G429E in humans) was only partially inactivating as RBL cells retained ˜25-30% of WT lytic activity. Based on the result of our RBL assays, one could predict that CTL expressing equal quantities of T224W- and G428E-perforin would have some residual but markedly reduced cytotoxic activity. In fact, the NK cells of Patient #5 did exhibit ˜15% lytic activity of control samples. The concordance of our data with the clinical findings in this case provides evidence that our experimental approaches should provide a robust basis for understanding other perforin mutations identified in HLH.

### Example 4

### Functional Analysis of Two Putative Polymorphisms (A91V and N252S) and 22 Misense Perforin Mutations Associated with Familial Hemophagocytic Lymphohistiocytosis

A. Construction of Mutated Perforin cDNAs

Mouse perforin cDNA cloned in pKS(+) Bluescript was mutated using the Transformer or QuickChange kits according to manufacturer's instructions (Stratagene) (oligonucleotide primer sequences provided on request). To avoid confusion in comparison to clinical cases, we have used the amino acid numbering of human perforin throughout this study. The relative positions of mutated residues are identical in the human and the mouse forms of the protein.

The WT or mutated perforin P39H, G45E, V50M, D70Y, C73R, A91V, W95R, G149S, F157V, V183G, G220S, T221I, H222R, H222Q, 1223D, R232C, R232H, N252S, E261K, C279Y, R299C, D313V, R361W and Q481P were cloned into the pIRES2-EGFP expression plasmid (BD Biosciences Clontech). Two allelic substitutions found in the flounder, R232S and Q481E, were similarly expressed. Each perforin cDNA was sequenced in full on both strands to check the fidelity of site-directed mutagenesis. The resultant expression plasmids were purified using the Qiagen Maxi-kit.

B. Transient Transfection of RBL Cells

Fcε-expressing RBL cells were cultured and transiently transfected as detailed in Example 3B above. BGFP-expressing cells were collected 18-20 hours later using flow cytometry (FACStar, Beckton-Dickinson). Numerous reports indicated the lack of perforin expression in NK cells of HLH patients, suggesting inherent instability of the mutated proteins. To address the issue, and given the large number of samples analysed, we had to be able to reliably compare the levels of expression of perforin variants. Therefore, prior to sorting transfected cells, the FACStar flow cytometer was calibrated by using CalibRITE FITC-labelled fluorescent beads (Beckton-Dickinson). We found that this approach provided us with reproducible levels of WT perforin expression and comparable cytotoxicity, on a day-to-day basis.

The cytotoxicity of RBL cells was analysed using Jurkat T cells as targets in a 4-hour ⁵¹Cr release assay as described in Example 3 above. Cell lysates from transiently-transfected RBL cells were resolved on a 10% SDS-PAGE (Tris-Glycine) gel, which was then analysed for perforin or tubulin expression by immunoblotting with P1-8 anti-perforin, or anti-tubulin antibodies, followed by the secondary HRP-linked anti-rat or anti-mouse immunoglobulin. The signal was detected by chemiluminescence (Amersham-Pharmacia).

C. Results

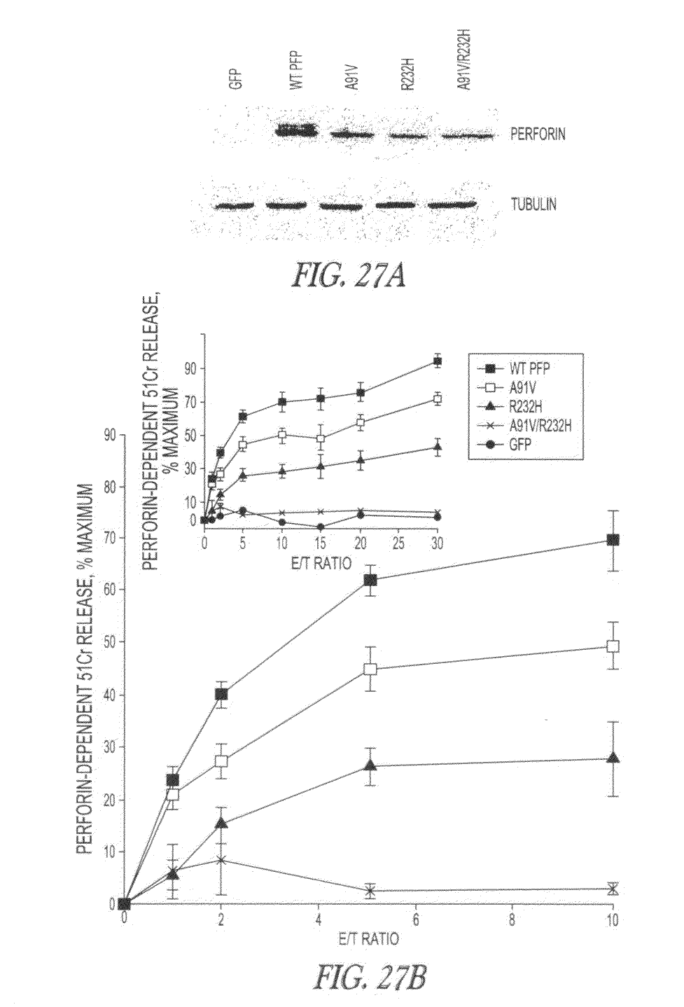

In this experiment, we undertook a functional analysis of 22 suspected HLH-causing missense mutations of PRF1 that map to various perforin domains, as listed in FIG. 26. To analyse the impact of the mutations on perforin function in isolation from potential defects in other loci, we expressed WT or mutated perforin in RBL cells as detailed earlier in Example 3, then ascertained their ability to lyse Jurkat target cells to which they were conjugated. Using this approach, we were able to discriminate likely pre-synaptic and post-synaptic dysfunctions of the various perforin molecules. We also performed a detailed analysis of two alterations of the perforin sequence that have thus far been considered to be PRF1 polymorphisms, A91V and N252S.

i) A Functional Analysis of the Suspected Perforin Polymorphism, A91V.

On the basis that it took about twice as many RBL cells to achieve a given level of target cell death, the cytotoxic activity of A91V perforin was consistently reduced by approximately 50% compared to WT FIG. 27). By the same criterion, R232H perforin was slightly less active than A91V, and generated approximately 30% of WT perforin activity. Importantly, the doubly mutated A91V/R232H protein was completely inactive.

Analysis of protein expression levels by Western blot revealed reduced expression of A91V, R232H and, to a greater extent A91V/R232H perforin, compared to the WT protein. These observations suggested that both mutations affected the folding and stability of perforin, and were likely to impact negatively on its cytotoxicity in the RBL assay. We also produced recombinant human A91V and WT perforin using the baculovirus expression system, as described in Example 3F above. We found that the lytic activity of A91V was reduced to <10% that of WT perforin (data not shown here). In addition, purified A91V was functionally unstable, in that its lytic activity rapidly diminishing to undetectable levels after 48 hours of storage at 4° C. By comparison, WT perforin was stable under these storage conditions for several months. On this basis, we propose that the A91V substitution results in protein misfolding that is most likely responsible for its reduced stability in RBL cells. This instability was augmented in the case of perforin purified from baculovirus-infected insect cells, possibly due to the absence of appropriate intracellular chaperone(s) in insect cells, and/or the altered redox environment. As a whole, the above assays indicated that the A91V substitution is an unusual type of PRF1 polymorphism in that it has a high allele frequency, but clearly results in reduced stability and consequently, partial loss of perforin lytic activity. We propose that the level of cytotoxic activity of A91V may generally be substantial enough to prevent HLH provided the second allele is WT, or even when the mutation is inherited in the homozygous state, as is the case in 1-4% of healthy populations.

(ii) A Functional Analysis of the Suspected Perforin Polymorphism, N252S.

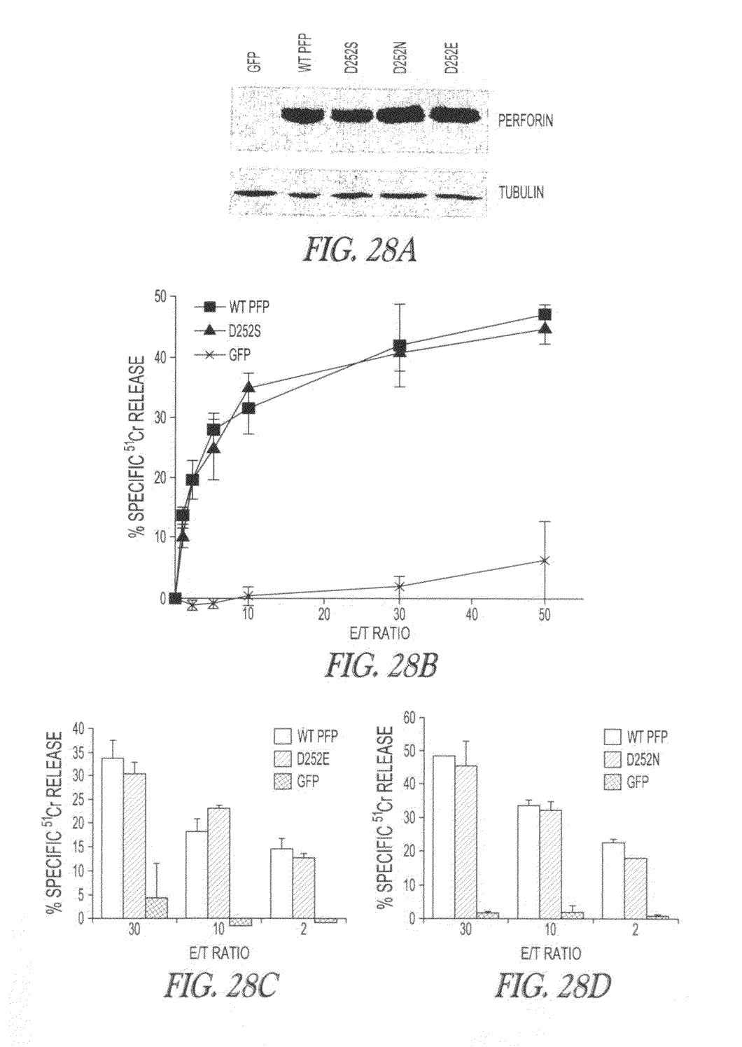

To elucidate the effect of the N252S substitution on perforin function, we generated several perforin mutations, D252N, D252E and D252S, and analysed their activity in the RBL cytotoxicity assay, the results of which are shown in FIG. 28. We found that all of these substitutions retained WT perforin activity. Assuming co-dominant expression, these observations suggested that an individual carrying the N252S allele and an inactivating mutation in their other PRF1 allele 28 would have ˜50% of normal perforin activity, consistent with the level of CTL activity observed by others in the HLH patients. Taken together, our data and epidemiological studies 9 indicate that the N252S substitution alone could not have been causative of disease, but rather, that an additional genetic defect(s) might have been responsible. We therefore concluded that N252S probably represents a true PRF1 polymorphism.

(iii) Functional Analysis of Missense Mutations Associated with HLH.

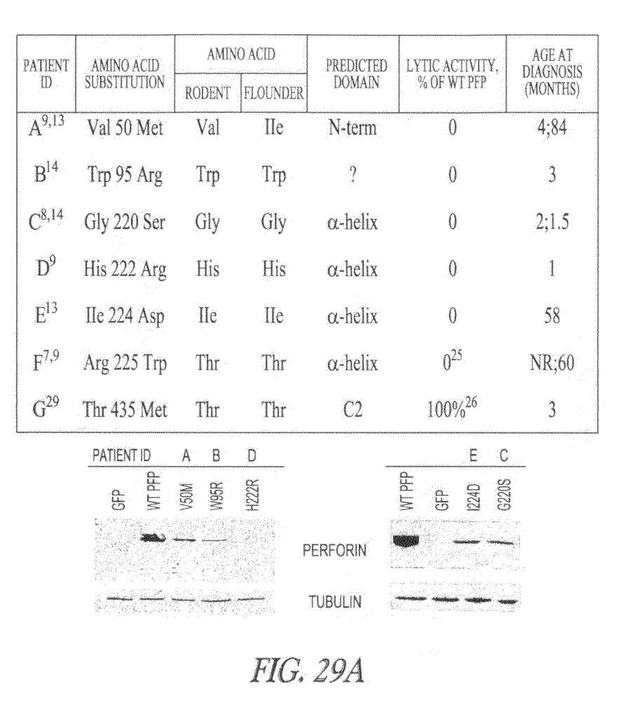

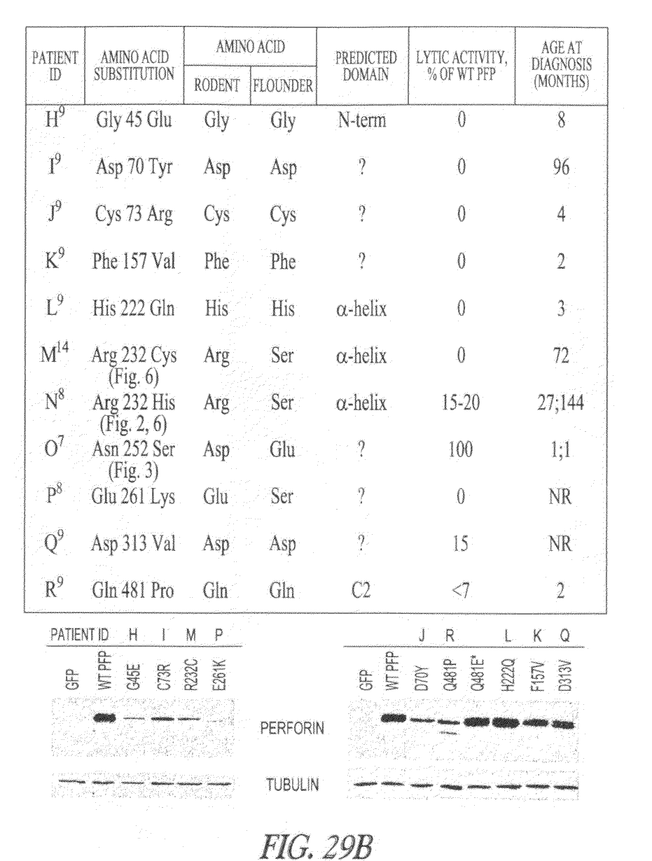

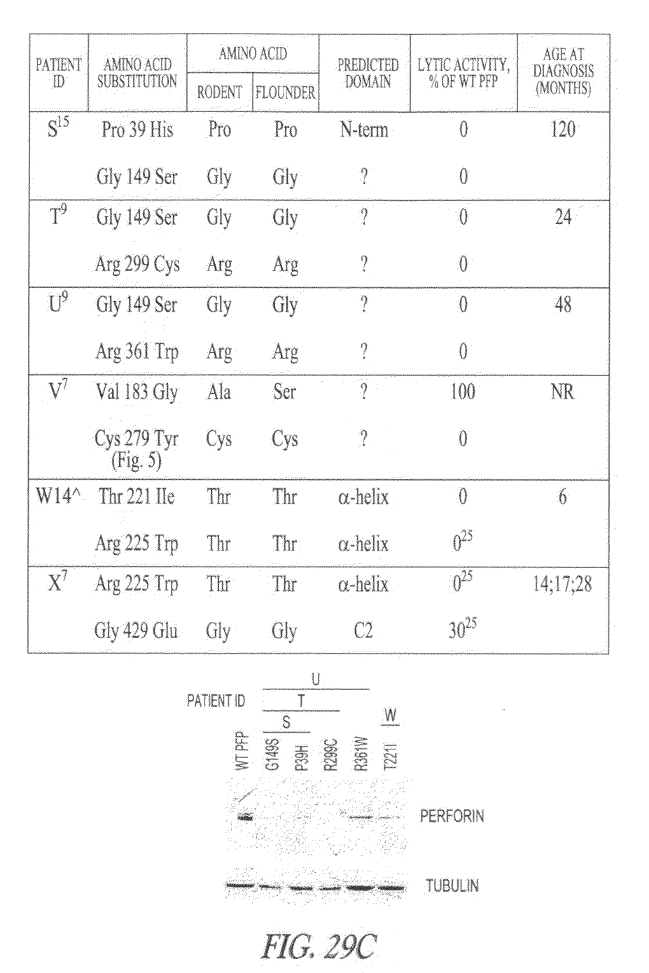

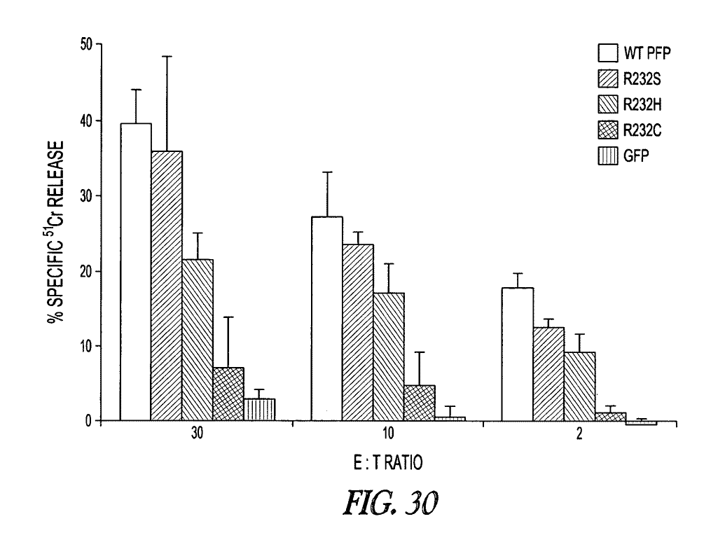

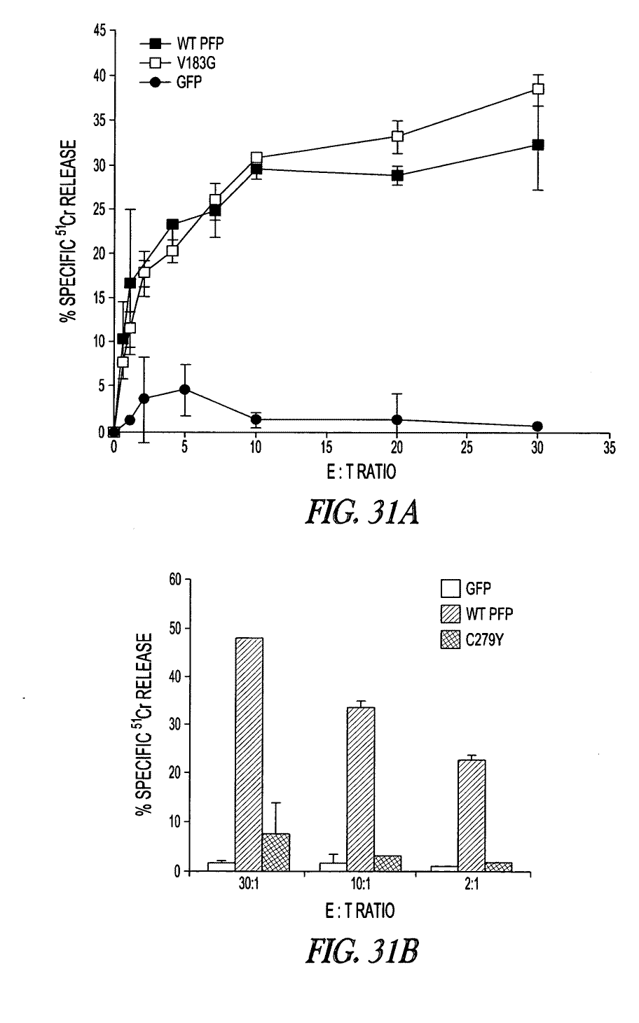

In the current study, we grouped perforin mutations according to the combinations of alleles reported in various HLH patients. FIG. 29A shows a summary of the results for alleles found in homozygous patients; FIG. 29B shows the corresponding data for alleles co-expressed with a null mutation (usually a truncation) of perforin, while FIG. 29C refers to alleles identified only in compound heterozygous patients with missense mutations in both alleles. This approach was chosen so that wherever possible, our findings might be usefully applied to the interpretation of corresponding clinical reports. We began our analysis of missense mutations by investigating whether a given mutation resulted in a pre-synaptic or post-synaptic dysfunction. The analysis of expression levels in RBL cells revealed that the majority of perforin mutations result in unstable/unfolded protein. Thus, according to Western blot analysis, perforin with the mutation P39H, G45E, G45R, V50M, D70Y, W95R, G149S, G220S, T221I, H222R, R232C, R232H, E261K, C279Y, R299C, R361W or Q481P was undetectable or greatly reduced in RBL cells compared to WT. It is likely that pre-synaptic defects of the mutated proteins were related to their misfolding or abnormal trafficking, leading to degradation. All of the unstable perforin variants had minimal detectable cytotoxic activity in the RBL cell-based ⁵¹Cr release assay (FIG. 29A-C). We also engineered amino acid substitutions R232S (FIG. 30) and Q481E (FIG. 29B), to mirror residues found in the corresponding position in flounder perforin. Unlike R232H (see also FIG. 27), R232S had normal activity, whereas R232C (reported in one HLH patient) 14 also had severely diminished function (FIG. 30). Flounder Q481E perforin also had WT expression levels (FIG. 29B) and activity (data not shown). Another grouping of perforin mutations analysed here were expressed quite differently from those described above. Contrary to clinical reports showing poor expression, V183G (FIG. 31) and H222Q perforin were expressed in RBL cells at a level equivalent to WT (FIG. 29B), and the expression levels of C73R, F157V and D313V perforin were only marginally reduced (FIG. 30B). Subsequently we used the ⁵¹Cr release cytotoxicity assay to analyse the cytotoxic properties of these mutated perforins. We were surprised to find that the lytic activity of V183G perforin, which has been implicated in HLH, was indistinguishable from that of the WT protein (FIG. 31). We concluded that the V183G mutation was unlikely to play a causative role in HLH for patient V (FIG. 29C), even though the second allele had an inactivating C279Y substitution. Given our experimental observations and the lack of amino acid conservation, we postulate that the V183G substitution is a true polymorphism of PRF1, and HLH in the corresponding patient was likely to be caused by some other mechanism independent of perforin. In addition, perforin mutations did not appear to have an appreciable ‘dominant negative’ effect on the function of WT perforin, as this property would be expected to affect perforin function in the patients' parents. Mutation of the conserved histidine, H222Q, resulted in normal expression of perforin in RBL cells, but the transfected RBL cells had no detectable cytotoxic activity (data not shown). Similar results were observed with non-conservatively substituted residues C73R, F157V and D313V mutations, whose expression levels in RBL cells were only slightly reduced compared to the WT perforin.

In conclusion, we have presented a comprehensive functional analysis of the missense mutations and polymorphisms of PRF1 thus far reported in association with HLH. Our data indicate that the instability of mutated perforin is a more common cause of perforin-related HLH than post-synaptic dysfunction. We established that the A91V mutation is an unusual case of “polymorphism” in that it significantly affects the stability and cytolytic activity of perforin, most likely due to incorrect folding of the protein. The fact that A91V is carried by a significant proportion of the healthy population in the homogygous state is in keeping with our experimental findings that this substitution nonetheless retains a significant proportion of WT function.

### Example 5

### Screening for Compounds with Perforin Inhibitor Activity

A. Reagents

Reagents used in this study are as follows:

HEPES (Sigma Aldrich Cat No. H-4034)

NaCl (BDH Cat No. 10241.45)

CaCl₂.2H₂O (BDH Cat No. 10070.44)

BSA (Sigma Aldrich Cat No. A-2153)

Polyoxyethylene Sorbitan Monolaurate (Tween 20; Sigma Aldrich Cat No. P-7949)

Triton X-100 (Sigma Aldrich)

Perkin Elmer SpectraMax 384-well plates (Cat No. 6007849)

B. Study Protocol

(i) Summary of Assay

| | |
|---|---|
| Target: | Perforin (mouse) |
| Target category: | Lytic protein |
| Assay associated with project(s): | |
| Assay technique: | Cell absorbance assay |
| Assay format: | 384-well |
| Alt. assay techniques | |
| investigated/Status | |
| Alternative assay(s) optimized: | |
| Enzyme: | Stock pooled purified perforin |
| | (~250 μg/ml) @4° C. |
| Assay substrate: | Sheep erythrocytes |
| Contact persons: | Annette De Bono-PMCI (9656 3725) |
| Date of delivery | Aug. 01, 2004 |

(ii) Assay Kinetics and Characteristics:

| | |
|---|---|
| [Enzyme] or dilution factor | ~1.5 nM (0.1 μg/ml) final conc. |
| | (1:2500) |
| [Substrate] (μM): | 10′ erythrocytes/well |
| Substrate kinetics (Km and Vmax): | N/A |
| [ATP] (for kinases): | N/A |
| ATP kinetics (for kinases, | N/A |
| Km and Vmax): | |
| Assay incubation time (min): | 15 min |
| Assay time linearity (min): | 10-15 min |
| S/B | 12-16 |
| Z′-factor | 0.8-0.9 |
| Final [DMSO] in assay (%): | 0.2% |
| DMSO tolerability: | Insignificant inhibition at 1% DMSO |
| Reference inhibitors tested | |
| (IC₅₀ in nM): | |
| Stability of enzyme solution: | 2-4 hr at 22° C. results in no loss of |
| | activity |
| Light sensitivity: | none |

(iii) Assay Reagents and Materials:

| | Content | Source | Comments |
|---|---|---|---|
| Buffer A | 10 mM HEPES, pH 7.4 | Sigma H-4034 | RT |
| | 150 mM NaCl | BDH 10241.45 | RT |
| | 0.01% BSA (fraction V) | Sigma A-2153 | Fresh |
| | 0.01% Tween20 | Sigma P-7949 | RT |
| Enzyme | 1:500 in buffer A | from PMCI | RT |
| solution | | | |
| RBC Buffer | buffer A plus | BDH 10070.44 | |
| | 1.875 mM CaCl₂ | | |
| Cells | 2.5 × 10⁸ cells/ml in | Sheep RBCs from | RT |
| working | RBC Buffer (10⁷ | UniMelb Vet School | |
| solution | cells/well). | | |
| 0% Control | 10 μl of Buffer A only | | |
| 100% | 10 μl of 2.5% Triton X- | Sigma X-100 | |
| Control | 100 | | |
| Plate | 384-well clear, flat | Packard | |
| | bottom | SpectraMax 384 | |
| | | (Cat no. 6007849) | |
| Reader | Envision (Perkin Elmer) | ABS@650 protocol | |

(iv) Assay Method:

0.1 μl compound/DMSO was added to 10 μl of 0.5 μg/ml perforin in buffer A or controls, respectively, using MiniTrak 1× using “Perforin-pintool transfer” method, with at least 30 min pre-incubation with compound routinely. 40 μl of sheep RBCs was then added in RBC Buffer using Zymark “Perforin2v4” method utilizing MultiDrop. Lysis of the sheep RBC results in a change in turbidity of the reaction mixture, whereas inhibition of cell lysis results in reduction or abolition of the change in the turbidity reading. As the inhibitor compounds were routinely dissolved in DMSO, the same concentration of DMSO was used as a negative control for the inhibition of perforin. In the wells where DMSO was used, perforin lysis was not inhibited, and the change in turbidity was equivalent to that observed in the absence of DMSO or inhibitor compounds.

Samples were initially read (t=0 min) at an absorbance of 650 nm (in Envision; using an Envision reader, automation ABS@650 nm), incubated for 15 min at 37° C., then read at an absorbance of 650 nm (in Envision) to assess a change in turbidity of the reaction mixture.

C. Experimental Procedures

The primary perforin-mediated lysis assay is based on the measurement of cell turbidity detected by absorbance measurements at a wavelength of 650 nm. Thus, the assay determines the potency of compounds by inhibition of perforin-mediated lysis of sheep RBC. Lysis of the sheep RBC results in a change in turbidity of the reaction mixture, whereas inhibition of cell lysis results in reduction or abolition of the change in the turbidity reading. As the inhibitor compounds were routinely dissolved in DMSO, the same concentration of DMSO was used as a negative control for the inhibition of perforin. In the wells where DMSO was used, perforin lysis was not inhibited, and the change in turbidity was equivalent to that observed in the absence of DMSO or inhibitor compounds.

(i) Primary Screen

The primary screen was performed at a final concentration of compound of 20 μM. Compounds were assayed as single-points.

(ii) Secondary Screen—Compound Dilution Plate Format

A 5-point dose-response was established with stock compound (controls in columns #23 and #24) serially diluted (changing pipette tips for each dilution series) into V-shaped, polypropylene 384-well plates (Matrical, Cat No. MP101-3-PP), from which 0.5 μl of diluted compound was dispensed per well of single assay plates (SpectraMax clear, flat bottom, 384-well plates, Perkin Elmer Cat No. 6007849), i.e. up-to 64 compounds tested per single assay plate.

(iii) Compound Concentrations

| | Compound concentration | Final compound |
|---|---|---|
| Dilution no. | in 100% DMSO | concentration in assay |
| 1 | 10000 μM | 100 μM |
| 2 | 2000 μM | 20 μM |
| 3 | 400 μM | 4 μM |
| 4 | 80 μM | 0.8 μM |
| 5 | 16 μM | 0.16 μM |

(iv) Data Analysis

The data obtained from each replicate experiment were analysed using the software ActivityBase™, version 5.0.10 (ID Business Solutions Ltd). The molar concentration of test compound producing 50% inhibition (IC₅₀) of the perforin-mediated cell lysis was derived utilising the MS Excel-based program XLfit (verson 3.0.5) to fit data to a 4-parameter logistic function of the form:

y=A+(B−A)/(1+((C/x)̂D))

wherein:

A is the bottom plateau of the curve i.e. the final minimum y value;

B is the top of the plateau of the curve i.e. the final maximum y value;

C is the x value at a y value of 50%. This represents the log IC₅₀ value when A+B=100;

D is the Hill slope factor. In this model a positive value is returned when y decreases with increasing x;

X is the original known x values; and

Y is the original known y values.

D. Results

(i) Primary Screen Data

| | ≧60% inhibition | | IC₅₀ ≦ 20 μM in |
|---|---|---|---|
| No. of | of lysis in primary | IC₅₀ ≦ 100 μM in | secondary |
| compounds | screen | secondary screen | screen |
| 101,024 | 612 | 333* | 132* |

(ii) Secondary Screen

All 612 compounds identified in the primary screen were subsequently tested at 100 μM, 20 μM, 4 μM, 0.8 μM and 0.16 μM using the same methodology as in the Primary screen, and the 384 well format. Of the 612 compounds, it was confirmed that 333 reproducibly inhibited sheep RBC lysis of mouse perforin with a IC50<100 mM. Of the 333 compounds, 132 were observed to have the greatest potency, defined as inhibiting lysis of sheep RBC with an IC50<20 μM.

(iii) Tertiary Screen

129 of the 132 compounds with an IC50<20 mM were tested for a third time for inhibition of lysis of mouse perforin. On this occasion, each inhibitor was tested at 100 mM, 25 mM, 5 mM and 1 mM (see Table below). The methodology for the sheep RBC lysis assay was varied as follows:

Compound/DMSO was added to perforin or controls and pre-incubated for 30 minutes in the wells of a 96-well V-bottom plate. All reagents were prepared as described above. Sheep RBC (prepared as described above) were then added and the plate was incubated for 15 minutes at 37° C. The plate was then centrifuged at 1500 rpm for 3 minutes at ambient temperature. Supernatant was collected from each test well with a pipette, and hemoglobin release was quantitated by measuring absorbance at 541 nM. Maximum hemoglobin release from the RBC was determined by resuspending the same number of sheep RBC in the same volume of distilled water, The negative control for lysis consisted of incubating the same number of sheep RBC in the same volume of buffer A without perforin. The percentage inhibition of lysis for each compound is shown in the Table.

% Inhibition by Lysis

| Compound ID No | 100 μM | 25 μM | 5 μM | 1 μM |
|---|---|---|---|---|
| 81690 | 99.4 | 101.3 | 102.6 | 14.6 |
| 83430 | 100.3 | 101.2 | 100.4 | 4.4 |
| 85062 | 99.24 | 97.7 | 96.5 | −22.6 |
| 86745 | 98.9 | 91.5 | 101.1 | −6.2 |
| 86830 | 98.1 | 100.6 | 102.6 | 10.8 |
| 87634 | 94.4 | 99.1 | 102.6 | 53.7 |
| 90683 | 100.1 | 101.6 | 103.26 | 12.5 |
| 91500 | 96.7 | 72.9 | 103.3 | 13.2 |
| 91507 | 32.6 | 16.8 | 74.7 | 14.1 |
| 93511 | 96.7 | 100.7 | 102.5 | 47.5 |
| 93694 | 99.4 | 101.2 | 103.5 | 0.08 |
| 95199 | 96.5 | 100.9 | 102.7 | 9.9 |
| 96634 | 87.5 | 87.3 | 100.1 | 85.2 |
| 97497 | 100.8 | 101 | 102.4 | 34.8 |
| 97753 | 93.6 | 100.1 | 91.9 | 38.1 |
| 98602 | 97.9 | 101.3 | 103.1 | 30.2 |
| 98714 | 99.1 | 101.2 | 102.8 | −11.4 |
| 98796 | 99.6 | 101.3 | 103.5 | 32.2 |
| 98853 | 98.7 | 101.5 | 103 | 56.9 |
| 98890 | 100.1 | 101.4 | 103.1 | 43.6 |
| 99593 | 97.9 | 100.7 | 102.8 | 52.7 |
| 99719 | 97.9 | 101 | 102.9 | 48.7 |
| 99746 | 96.6 | 102 | 102.9 | 91.1 |
| 100904 | 93.3 | 54.4 | 85.8 | 20.8 |
| 101334 | 59.8 | 88.1 | 20.9 | 1.8 |
| 102196 | 98.7 | 100.4 | 96.4 | 7.7 |
| 81459 | 95.9 | 100.7 | 72.4 | −20.7 |
| 7816 | 99.8 | 101.7 | 102.9 | 18 |
| 77033 | 82.2 | 88.2 | 64.3 | 8.8 |
| 56384 | 99.4 | 100.7 | 100.8 | 10.3 |
| 53476 | 98.7 | 101.3 | 98.6 | 90.2 |
| 54349 | 33.5 | 97.4 | 102.9 | 26.8 |
| 53700 | 80 | 91.7 | 100.8 | 98.5 |
| 51550 | 97.5 | 100 | 102.8 | 24.8 |
| 51346 | 99 | 101.4 | 102.9 | 86.2 |
| 35654 | 96.8 | 99.9 | 102.4 | 94.4 |
| 34488 | 99.7 | 100.9 | 101.1 | 98.2 |
| 34231 | 99.9 | 101.4 | 102.9 | 86.7 |
| 33744 | 93.7 | 92.8 | 81.9 | −36.9 |
| 33465 | 99.7 | 100.1 | 102 | 21.9 |
| 32846 | 96.7 | 97.1 | 99.1 | 86.3 |
| 32845 | 85.5 | 99.3 | 101.8 | −2.3 |
| 31622 | 97.9 | 100.3 | 102.1 | 33.9 |
| 17306 | 96.7 | 100.3 | 102.6 | 94.7 |
| 17020 | 97.6 | 100.7 | 102.5 | 3.3 |
| 16612 | 95.8 | 102.2 | 101.6 | 1.6 |
| 14621 | 95.4 | 96.8 | 102.6 | 35.7 |
| 14279 | 99.7 | 104.1 | 102.1 | 99.4 |
| 13729 | 97.8 | 99.9 | 101.4 | 26.6 |
| 13655 | 98.5 | 101.3 | 101.9 | −4.9 |
| 5857 | 99.1 | 102.6 | 102.6 | 77.8 |
| 49391 | 99.6 | 103.4 | 103.1 | 95.1 |
| 46553 | 98.4 | 99.2 | 103.2 | 97.4 |
| 44146 | 99.4 | 100.4 | 101.8 | 76.4 |
| 40217 | 98.1 | 99.1 | 102.6 | 79.7 |
| 40021 | 98.8 | 49.5 | 100.2 | 10.8 |
| 39822 | 97.7 | 93 | 102.9 | 78.1 |
| 37011 | 37.3 | 78.3 | 96.4 | 94.4 |
| 37003 | 97.5 | 101.1 | 101.9 | 22.2 |
| 36892 | 99.2 | 96.6 | 42.1 | 7.5 |
| 36837 | 99.9 | 98.1 | 101.2 | −8.5 |
| 88403 | 98.8 | 101.5 | 98.3 | 0.2 |
| 88082 | 94.3 | 102.2 | 101.2 | 0.08 |
| 88071 | 84.8 | 92.7 | 97.9 | 91.6 |
| 86792 | 99.1 | 97.9 | 97.5 | 32.3 |
| 86737 | 99.7 | 97.7 | 96.9 | 50.5 |
| 86671 | 96.7 | 93.8 | 101.6 | 83.9 |
| 85851 | 98.2 | 98.1 | 91.1 | 55.9 |
| 85368 | 99.5 | 103.6 | 98.6 | 16 |
| 84575 | 96.02 | 101.4 | 86.8 | −4.3 |
| 83514 | 96.3 | 101.6 | 97.98 | −8.3 |
| 83439 | 98.4 | 103.5 | 99.2 | 89.5 |
| 82708 | 98.7 | 101.6 | 101 | 98.5 |
| 82465 | 91.2 | 97.5 | 102.1 | −10 |
| 80405 | 83.7 | 100.8 | 101.9 | 5.8 |
| 80377 | 97.6 | 103.5 | 98.8 | 22.7 |

% Inhibition by Lysis

| Compound ID No | 100 μM | 25 μM | 5 μM | 1 μM |
|---|---|---|---|---|
| 77708 | 88.5 | 94.6 | 102.5 | 10.2 |
| 77367 | 96.9 | 99.9 | 59.4 | 96.5 |
| 76429 | 86.3 | 87.7 | 75.3 | 3.4 |
| 75689 | 98.2 | 99.5 | 102.9 | 92.2 |
| 74871 | 98.1 | 102.4 | 101.6 | 11.2 |
| 74470 | 92.9 | 103.4 | 100.9 | 5.4 |
| 74401 | 97.8 | 104 | 102.1 | 5.9 |
| 74043 | 93.2 | 103.8 | 102.8 | 36.4 |
| 73303 | 96.6 | 102 | 68.9 | 31.4 |
| 72176 | 99.5 | 104.2 | 100.9 | 7.2 |
| 71998 | 99.8 | 104.5 | 98.9 | 15.9 |
| 69026 | 99.5 | 104.6 | 98.4 | 19.3 |
| 67186 | 97.3 | 101.9 | 80.2 | 15.4 |
| 65683 | 89.5 | 103.2 | 53.3 | 56.6 |
| 64537 | 92.2 | 96.6 | 88.3 | 9.4 |
| 64234 | 67.1 | 87.7 | 3.9 | 13.7 |
| 60658 | 88.4 | 105.8 | 102.2 | 49.6 |
| 59160 | 75.1 | 95.6 | 98.3 | 23.9 |
| 58388 | 25.3 | 106.1 | 102.3 | 11.2 |
| 57871 | 105.6 | 106.7 | 76.1 | 15.3 |
| 57806 | 105 | 106.5 | 103.4 | 101.3 |
| 57777 | 105.1 | 106.6 | 103.5 | 99.1 |
| 56930 | 106.4 | 106.9 | 103.2 | 105.8 |
| 34488 | 106.3 | 106.6 | 103.4 | 106.1 |
| 33465 | 105.7 | 105.7 | 102.9 | 60.7 |
| 17020 | 106.1 | 106.7 | 56.3 | 64.8 |
| 14279 | 106.4 | 106.9 | 97.4 | 97.8 |
| 13655 | 75.1 | 74.7 | −13.4 | 19.3 |
| A1 | 84.2 | 77.8 | 22.4 | 70.6 |
| A2 | 105.9 | 103.5 | 98.1 | 105.6 |
| A3 | 34.7 | 94.7 | 55.4 | 93.6 |
| B1 | 13.9 | 95.3 | 14.6 | 80.9 |
| B2 | 102.6 | 106.2 | 101.5 | 104.8 |
| B3 | 29.7 | 44.3 | 22.7 | 51.2 |
| C1 | 89.1 | 102.9 | 45.9 | 101.3 |
| C2 | 105.6 | 105.3 | 100.7 | 104.3 |
| C3 | 105.7 | 92.8 | 1.1 | 99.8 |
| D1 | 106.3 | 105.3 | 79.9 | 93.9 |
| D2 | 105.4 | 106.5 | 98.8 | 78.6 |
| D3 | 102.1 | 105.5 | 24.1 | 85.4 |
| E1 | 104.2 | 106.1 | 99.9 | 93.7 |
| E2 | 103.3 | 106.5 | 58.2 | 97.2 |
| E3 | 105 | 106.5 | 63.8 | 98.4 |
| F1 | 19.5 | 44.6 | 19.6 | 83.6 |
| F2 | −2.2 | 40.6 | 11.6 | 81 |
| F3 | 70.3 | 100.6 | 42.8 | 74.2 |
| G1 | 101.8 | 105.8 | 50.1 | 95.3 |
| G2 | 101.2 | 106.1 | 100.6 | 104.7 |

% Inhibition by Lysis

| Compound ID No | 100 μM | 25 μM | 5 μM | 1 μM |
|---|---|---|---|---|
| G3 | 103.4 | 103.5 | 98 | 104.4 |
| H1 | 100 | 101.6 | 99.1 | 104.9 |
| H2 | 101.6 | 104.6 | 58.8 | 101.3 |

The results demonstrate that at 100 μM and 25 μM, most compounds are able to inhibit perforin-induced red blood cell lysis, and when used at 1 μM a number of them are still quite potent. Approximately 30% of the compounds are still potent when used at

(iii) Sheep Red Blood Cell Assay in the Presence of 0.1%, 0.5% and 1% BSA, with Compounds at 20 μM.

Of the 129 compounds, we chose 46 of the most potent compounds (at 20 μM), along with negative controls, and carried out an SRBC lysis assay in the presence of varying concentrations of bovine serum albumin (BSA). We found that 22 of the compounds were still able to inhibit mouse perforin by at least 60%, when BSA was present at 0.1%.

% Inhibition by Lysis

| Compound ID No. | 0.1% | 0.50% | 1.00% |
|---|---|---|---|
| 93511 | 38.4 | −6.1 | −9.2 |
| 96634 | 91 | 9 | −7 |
| 98853 | 32 | 30 | 34 |
| 99746 | 4.5 | −4 | −3 |
| 53476 | 57 | 43 | 22 |
| 53700 | −11 | −10 | −10 |
| 51346 | 77 | 28 | 39 |
| 35654 | 30 | −3 | 1.3 |
| 34488 | 7 | 2 | 3 |
| 34231 | 94 | 72 | 44 |
| 32846 | 91 | 64 | 11 |
| 31622 | 72 | 80 | 62 |
| 17306 | −5 | 2 | −10 |
| 17020 | 93 | 61 | 64 |
| 14279 | 96 | 52 | 25 |
| 5857 | 101 | 102 | 92 |
| 49391 | 102 | 107 | 108 |
| 46553 | 100 | 100 | 95 |
| 40217 | 97 | 109 | 107 |
| 39822 | 82 | 45 | 33 |
| 88071 | 7 | 2 | −16 |
| 86792 | 13 | 34 | 37 |

% Inhibition by Lysis

| Compound ID No. | 0.1% | 0.50% | 1.00% |
|---|---|---|---|
| 86671 | 59 | 63 | 84 |
| 85851 | 76 | 67 | 15 |
| 84575 | 32 | −12 | 20 |
| 83439 | 100 | 93 | 88 |
| 82708 | 0 | −2 | 7.7 |
| 77367 | 94 | 83 | 50 |
| 75689 | 29 | 18 | 19 |
| 74871 | −3 | 25 | 8.5 |
| 67186 | 37 | 45 | 28 |
| 64537 | 88 | 64 | 58 |
| 62030 | 74 | 19 | 7 |
| 57871 | 51 | 22 | 27 |
| 57806 | 83 | 14 | 4 |
| 57777 | 60 | 47 | 27 |
| 56930 | 109 | 113 | 115 |
| 34488 | 27 | 15 | 9 |
| 33465 | 91 | 49 | 32 |
| A2 | 61 | 6 | 27 |
| B1 | 31 | −1 | — |
| B3 | 17.8 | 5.4 | 1.5 |
| D1 | −2.5 | 48 | 74 |
| D2 | 5 | 10 | 7 |
| F1 | 16.9 | 11.4 | 1.3 |
| F3 | 17.8 | 5.4 | 1.5 |

(iv) Inhibition of Perforin on Nucleated (Jurkat) Cells in the Presence of 0.1% BSA in HE Buffer

The compounds which were still able to inhibit perforin lysis of sheep RBC by greater than 60% in the presence of 0.1% BSA were then tested for their ability to inhibit the lysis of nucleated cells (Jurkat T lymphoma cells), by ⁵¹Cr release assay in the presence of 0.1% BSA at 80 μM, 20 μM, 5 μM and 1 μM. The compounds were tested in HE buffer_or RPMI medium, and the data shown below are for HE.

| | | | | |
|---|---|---|---|---|
| Compound ID No. | 80 μM | 20 μM | 5 μM | 1 μM |

% Inhibition by ⁵¹Cr Release
on Jurkat

| | | | | |
|---|---|---|---|---|
| 93511 | 94.5 | 99.7 | 100.8 | 100.5 |
| 96634 | 91.8 | 92.8 | 91 | 77.2 |
| 99746 | 74.7 | 92.3 | 98.9 | 95.5 |
| 53476 | 98.2 | 101.3 | 82.3 | 13.1 |
| 53700 | 77.3 | 97.9 | 78.8 | 28.8 |

% Inhibition by ⁵¹Cr Release
on Jurkat pgk

| | | | | |
|---|---|---|---|---|
| 35654 | 76.1 | 88.2 | 53.2 | 2.8 |
| 34488 | −3.6 | −2.8 | −1.9 | −0.8 |
| 34231 | 98.2 | 105.5 | 93.3 | 56 |
| 32846 | 97 | 99.8 | 100.2 | 101.2 |
| 31622 | 59.2 | 17.1 | 2.7 | 5.6 |
| 17306 | 91.2 | 91.8 | 12 | 1.3 |
| 17020 | 88.4 | 12 | 1 | 0.2 |
| 14279 | 98.7 | 30.5 | −2.1 | −0.4 |
| 5857 | 96.1 | 99 | 88.3 | 61.1 |
| 49391 | 94.5 | 97.5 | 99.6 | 49.4 |
| 46553 | 95.6 | 95.3 | 97.3 | 96.7 |
| 40217 | 99 | 90.8 | 92.3 | 51.8 |
| 39822 | 97.5 | 96.6 | 25.1 | 8.2 |
| 88071 | 65.3 | 40.5 | −3.1 | −5.7 |
| 86792 | 98.7 | 93.9 | 46.2 | 6 |
| 86671 | 97.9 | 88.2 | 73.9 | 31.1 |
| 85851 | 84.2 | 33.4 | 10.6 | 6.2 |
| 83439 | 74.4 | 93.6 | 99.4 | 78.7 |
| 82708 | 38.1 | 30.7 | 5.8 | 2.9 |
| 77367 | 95.3 | 104.9 | 99.5 | 86.8 |
| 75689 | 76.4 | 65.3 | 30.3 | 3.4 |
| 74871 | 95.9 | 90 | 19.4 | 2.4 |
| 67186 | 0.1 | 3.8 | 7.8 | 6 |
| 64537 | 81.4 | 85.2 | 82.8 | 48.5 |
| 62030 | 88.6 | 88.5 | 41 | 3.5 |
| 57871 | 1.3 | −2.6 | 5.6 | 8.1 |
| 57806 | 97.3 | 100.3 | 96.9 | 37.4 |
| 57777 | 100.7 | 24.7 | 13.6 | 12.9 |
| 56930 | 90.3 | 91.9 | 98.8 | 74.3 |
| 34488 | 0.3 | 0.9 | 0.9 | −3.6 |
| 33465 | 62.6 | 10 | 5.8 | 6.6 |
| B3 | 4 | 5.6 | 12.4 | 13 |

The results show that at 80 μM, 30 of the 36 compounds inhibited perforin by 60% or greater; at 20 μM, 24 of the 36 compounds inhibited perforin by 60% or greater; at 5 μM, 17 of the 36 compounds inhibited perforin by 60% or greater; and at 1 μM, 9 of the 36 compounds inhibited perforin by 60% or greater.

(v) Inhibition of Perforin Lysis of Nucleated Jurkat Cells in the Presence of 0.1% BSA in RPMI Buffer

| Compound ID | % Inhibition by ⁵¹Cr Release on Jurkat | | | | |
|---|---|---|---|---|---|
| No. | 20 μM | 5 μM | 1.25 μM | 0.3 μM | .08 μM |
| 46553 | 98 | 96 | 90 | 87 | 48 |
| 96634 | 90 | 53 | 40 | 38 | 30 |
| 32846 | 100 | 100 | 86 | 53 | 20 |
| 05857 | 82 | 43 | 20 | 10 | 0 |
| 83439 | 22 | 34 | 10 | 7 | 10 |
| 56930 | 82 | 95 | 70 | 42 | 23 |
| 57806 | 93 | 50 | 40 | 40 | 32 |
| 49391 | 100 | 100 | 97 | 77 | 36 |
| 40217 | 100 | 93 | 62 | 43 | 25 |
| 93511 | 100 | 98 | 80 | 40 | 20 |
| 99746 | 100 | 92 | 55 | 18 | 10 |
| 53700 | 18 | — | — | — | — |
| 86671 | 83 | 40 | 16 | 20 | 8 |
| 64537 | 90 | 50 | 30 | 20 | 10 |
| 83430 | 98 | 70 | 40 | 22 | 10 |
| 35654 | 85 | 48 | 25 | 15 | 18 |
| 54376 | 98 | 70 | 30 | 18 | 10 |

(vi) Specificity of action—In order to test whether the inhibitors specifically inhibited perforin, or were also able to block the lytic function of the pneumococcal toxin pneumolysin (PLO), the inhibitors in the Table below were tested at 20 μM for the their ability to inhibit sheep RBC lysis induced by PLO. None has a significant inhibitory effect on PLO, indicating they acted specifically to inhibit perforin.

| | % Inhibition of |
|---|---|
| | PLO with |
| Compound | compounds |
| ID No. | at 20 μM |
| 81690 | −37.4 |
| 83430 | 26.7 |
| 85062 | −17.8 |
| 86745 | −11.4 |
| 86830 | −6.9 |
| 87634 | −14.8 |
| 90683 | 7.43 |
| 91500 | 7.29 |
| 91507 | 23 |
| 93511 | −17.8 |
| 93694 | 5.3 |
| 95199 | −1.7 |
| 96634 | −14.9 |
| 97497 | −6.4 |
| 97753 | −21.9 |
| 98602 | −5.8 |
| 98714 | 38.8 |
| 98796 | −34.7 |
| 98853 | −6 |
| 98890 | 1.82 |
| 99593 | −4.3 |
| 99719 | 19.3 |
| 99746 | −11.6 |
| 100904 | 5.5 |
| 101334 | −35.2 |
| 102196 | −13.7 |
| 81459 | 16.5 |
| 7816 | −12.8 |
| 77033 | 2.24 |
| 56384 | −12.9 |
| 53476 | 28.2 |
| 54349 | 15.6 |

| Compound | % Inhibition with |
|---|---|
| ID No. | PLO at 20 μM |
| 53700 | −21.4 |
| 51550 | 8.2 |
| 51346 | 13.8 |
| 35654 | −3.9 |
| 34488 | −3.3 |
| 34231 | 2.8 |
| 33744 | −14.4 |
| 33465 | 21.9 |
| 32846 | −25.4 |
| 32845 | −19.9 |
| 31622 | 12 |
| 31330 | −6 |
| 17306 | −1.8 |
| 17020 | 11.6 |
| 16612 | −0.5 |
| 14621 | 17.9 |
| 14279 | −8.11 |
| 13729 | −11.8 |
| 13655 | −13.9 |
| 5857 | 15.5 |
| 49391 | −5.7 |
| 46553 | −31.7 |
| 44146 | −43.1 |
| 40217 | 14.1 |
| 40021 | −18.1 |
| 39822 | 5.5 |
| 37011 | −10.02 |
| 37003 | 18.8 |
| 36892 | 1.05 |
| 36837 | −1.4 |
| 88403 | 0.91 |
| 88082 | 10.2 |
| 88071 | −22 |
| 86792 | 11.3 |
| 86737 | 2.7 |
| 86671 | −10.9 |
| 85851 | 8.73 |
| 85368 | −3 |
| 84575 | −2.7 |
| 83514 | −27.9 |
| 83439 | −5.7 |
| 82708 | −22.4 |
| 82465 | 0 |
| 80405 | −15.5 |
| 80377 | 9.2 |
| 77708 | 7.08 |
| 77367 | −9.5 |
| 76429 | −3.4 |

| Compound | % Inhibition with |
|---|---|
| ID No. | PLO at 20 μM |
| 75689 | 9.89 |
| 74871 | −5.2 |
| 74470 | −16.9 |
| 74401 | −11.6 |
| 74043 | −19.1 |
| 73303 | 6.4 |
| 71998 | −6.7 |
| 69026 | −13.6 |
| 67186 | −2.5 |
| 65683 | −13.7 |
| 64537 | −31.6 |
| 64234 | −4.7 |
| 62030 | −42.3 |
| 60658 | 12.7 |
| 59160 | −15.6 |
| 58388 | 3.5 |
| 57871 | −20.7 |
| 57806 | 15.7 |
| 57777 | 9 |
| 56930 | −19.6 |
| 34488 | 4.6 |
| 33465 | 0.8 |
| 17020 | 11.6 |
| 14279 | −8.11 |
| 13655 | −13.9 |

| | |
|---|---|
| A1 | 21.9 |
| A2 | −20.9 |
| A3 | −23.1 |
| B1 | −20.1 |
| B2 | −30.9 |
| B3 | −2.9 |
| C1 | 10.5 |
| C2 | −14.8 |
| C3 | −24.2 |
| D1 | −30.8 |
| D2 | −46.4 |
| D3 | −36.7 |
| E1 | −66.7 |
| E2 | −50.9 |
| E3 | −21.8 |
| F1 | −57.9 |
| F2 | −56.9 |
| F3 | −48.7 |
| G1 | −23.4 |
| G2 | −46.1 |
| G3 | −30.99 |
| H1 | −45.2 |
| H2 | −20.6 |

(vii) Inhibition of Mouse and Human Perforin in the Sheep Red Blood Cell Lysis Assay (Compounds Used at 1 μM)

All of the screening of perforin inhibitors described above was performed using mouse perforin. The compounds in the Table below were simultaneously tested for their ability to inhibit sheep RBC lysis in response to both mouse and human perforin.

| | |
|---|---|
| Mouse Perforin | Human Perforin |

| | |
|---|---|
| Compound ID No. | % Inhibition by Lysis at 1 μM |

| | | |
|---|---|---|
| 93511 | 23.9 | 94.4 |
| 96634 | 65.2 | 101.6 |
| 99746 | 34.4 | 102.2 |
| 53700 | 96.7 | 103.9 |
| 35654 | 47.3 | 101.9 |
| 34488 | 97.8 | 104.3 |
| 34231 | 27.5 | 102.9 |
| 32846 | 89.4 | 103.3 |
| 17306 | 41.6 | 98.7 |
| 46553 | 69.2 | 103.7 |
| 88071 | 18.4 | 80.2 |
| 82708 | 94.2 | 101.2 |
| 77367 | 93.1 | 93.3 |
| 75689 | 99.4 | 100.5 |
| 62030 | 99.8 | 103.80 |
| 57806 | 95.78 | 104.0 |
| 34488 | 97.7 | 104.3 |
| E1 | 1.4 | 22.2 |

The results demonstrate that each compounds is able to inhibit human perforin with approximately equal or even slightly greater potency than mouse perforin. For example, compound ID no. 53700, inhibits mouse perforin by 96.7%, and human perforin by 103.9%.

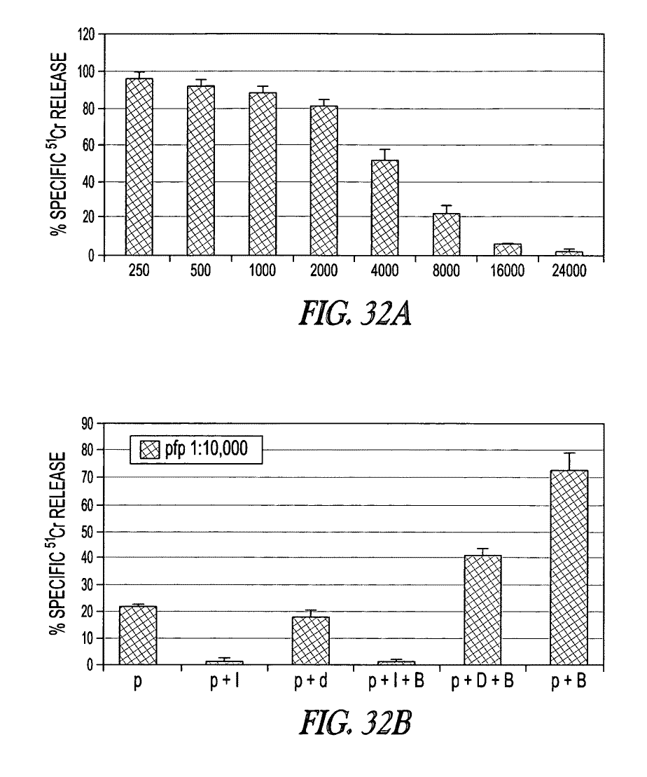

Inhibitor compound 46553 was then selected and assayed for its ability to block the synergy of perforin and granzyme B in inducing apoptosis of Jurkat cells. The results demonstrate that inhibitor compound 46553 completely blocked apoptosis of Jurkat cells (FIG. 32). Similar effects have also been seen with inhibitor compounds 34231, 77367 & 32846 (data not shown here).

Finally it is to be understood that various other modifications and/or alterations may be made without departing from the spirit of the present invention as outlined herein.

1. A method of identifying a compound that modulates expression of a perforin molecule, or a fragment or variant thereof that reacts with a perforin specific antibody, said method comprising the steps of:
providing a cell transfected with a retroviral vector capable of driving the expression of a perforin molecule or said fragment or variant thereof in a host cell transfected with said vector and wherein said vector comprises a polynucleotide encoding a perforin molecule or said fragment or variant thereof;
exposing the cell to a test compound; and
determining whether the test compound modulates the expression of the perforin molecule, or said fragment or variant thereof, in the cell.

2. A method of identifying a compound that modulates activity of a perforin molecule, or a fragment or variant thereof that reacts with a perforin specific antibody, said method comprising the steps of:
providing a cell transfected with a retroviral vector capable of driving the expression of a perforin molecule or said fragment or variant thereof in a host cell transfected with said vector and wherein said vector comprises a polynucleotide encoding a perforin molecule or said fragment or variant thereof;
exposing the cell to a test compound and a target cell; and
determining whether the test compound modulates the activity of the perforin molecule, or said fragment or variant thereof, upon the target cell.

3. The method according to claim 2, wherein the activity of the perforin molecule, or said fragment or variant thereof, upon the target cell is identified as the ability of the perforin molecule, or said fragment or variant thereof, to lyse the target cell.

4. The method according to claim 2, wherein the activity of the perforin molecule, or said fragment or variant thereof, upon the target cell is identified as the ability of the perforin molecule, or said fragment or variant thereof, to cause delivery of pro-apoptotic granzyme into the target cell or to induce apoptosis in the target cell.
